(19) United States
(12) Patent Application Publication
(10) Pub. No.: US 2017/0298314 A1
(43) Pub. Date: Oct. 19, 2017

(54) NANO-DROPLET PLATE

(71) Applicants: Research Foundation of the City University of New York, New York, NY (US); ARL Designs, LLC, New Providence, NJ (US)

(72) Inventors: Alan M. Lyons, New Providence, NJ (US); QianFeng Xu, Staten Island, NY (US)

(21) Appl. No.: 15/486,953

(22) Filed: Apr. 13, 2017

Related U.S. Application Data
(60) Provisional application No. 62/322,048, filed on Apr. 13, 2016.

(51) Int. Cl. C12M 1/12; B01L 3/00; C12M 1/00; C12M 1/32

(52) U.S. Cl. CPC C12M 25/06; C12M 25/01; C12M 23/12; C12M 23/22; B01L 3/5085; B01L 2300/0809; B01L 2300/0848; B01L 2300/0887; B01L 2300/12; B01L 2300/0896

(57) ABSTRACT

A low-cost method is provided for fabricating a nano-droplet plate with surface features having re-entrant (anvil-like) geometries capable of holding droplets of a precise, predetermined volume. Such structures are useful for a variety of applications, including cull culturing, high-throughput screening of therapeutics and as microwells.

### CROSS-REFERENCE TO RELATED APPLICATIONS

This application claims priority to, and is a non-provisional of, U.S. Patent Application 62/322,048 (filed Apr. 13, 2016), the entirety of which is incorporated herein by reference.

### STATEMENT OF FEDERALLY SPONSORED RESEARCH OR DEVELOPMENT

This invention was made with government support under grant number 1330949 awarded by National Science Foundation. The government has certain rights in the invention.

### BACKGROUND OF THE INVENTION

Precise dispensing of a large number of nanoliter droplets containing bioreagents is one of the most crucial steps for achieving reliable assay results and is highly desired for high throughput/content screening of new drugs or biomarkers. The need for precise dispensing of nanoliter (nL) quantities is especially acute when the source sample volume is limited, such as naturally occurring venom from snakes, spiders or other nature products. Conventional contact dispensing techniques do not offer sufficient precision for dispensing droplets less than 50 nL and microwell plates are too large to handle such small volumes. Dispensing volumes below 50 nL is challenging because the dispensing process is dominated by interfacial adhesion, and factors such as surface tension capillary forces and local microstructures affect the transferred volume. As a result, error increases significantly as the dispensed volume decreases from microliters to picoliters. Alternatively, noncontact dispensing systems create a jet of nL droplets and so relieves the tolerances imposed on positioning and substrate planarity, but increases the cost and complexity of the delivery system used, as well as subjecting the solution to high temperatures and/or shear forces that can damage large molecules and cells. Dispensing errors associated with jetting systems remain high, approximately ±10% with 20 nL droplets.

Although high energy barrier surfaces have been demonstrated, they are expensive to fabricate and are easily damaged due to their fragility. Also, the hydrophilic surface regions are too small (diameters less than 50 μm) to facilitate dispensing of individual droplets and would evaporate too quickly to be useful. Thus, there is a need for a surface that is inexpensive to fabricate, mechanically robust and with hydrophilic regions greater than 50 μm that can be used to precisely dispense arrays of nanoliter droplets.

The discussion above is merely provided for general background information and is not intended to be used as an aid in determining the scope of the claimed subject matter.

### BRIEF DESCRIPTION OF THE INVENTION

A low-cost method is provided for fabricating a nano-droplet plate with surface features having re-entrant (anvil-like) geometries capable of holding droplets of a precise, predetermined volume. Such structures are useful for a variety of applications, including cull culturing, high-throughput screening of therapeutics and as microwells.

In a first embodiment, a nano-droplet plate is provided. The nano-droplet plate comprises a rigid substrate with a planar surface; a plurality of elongated posts extending from the planar surface, each elongated post separated from adjacent elongated posts by a pitch distance of at least 1 mm. Each elongated post comprising: a bottom end directly connected to the planar surface, the bottom end having a bottom diameter; a top end opposite the bottom end, the top end comprising a planar top surface having a top diameter and an area of less than 50 square millimeters; and a middle section connecting the bottom end and the top end, the middle section having an upper half and a lower half proximate the top end and the bottom end, respectively, wherein the upper half contacts the planar top surface of the top end.

In a second embodiment, a cell culture system is provided. The cell culture system comprises a nano-droplet plate comprising a rigid substrate with a planar surface; a plurality of elongated posts extending from the planar surface, each elongated post separated from adjacent elongated posts by a pitch distance of at least 1 mm. Each elongated post comprising a bottom end directly connected to the planar surface, the bottom end having a bottom diameter; a top end opposite the bottom end, the top end comprising a planar top surface having a top diameter and an area of less than 50 square millimeters; a middle section connecting the bottom end and the top end, the middle section having an upper half and a lower half proximate the top end and the bottom end, respectively; wherein the upper half contacts the planar top surface of the top end. The cell culture system further comprises a well microplate comprising a plurality of wells, wherein the ratio of wells to elongated posts is 1:1 and the pitch distance is equal to a center-to-center well spacing of the plurality of wells.

This brief description of the invention is intended only to provide a brief overview of subject matter disclosed herein according to one or more illustrative embodiments, and does not serve as a guide to interpreting the claims or to define or limit the scope of the invention, which is defined only by the appended claims. This brief description is provided to introduce an illustrative selection of concepts in a simplified form that are further described below in the detailed description. This brief description is not intended to identify key features or essential features of the claimed subject matter, nor is it intended to be used as an aid in determining the scope of the claimed subject matter. The claimed subject matter is not limited to implementations that solve any or all disadvantages noted in the background.

### BRIEF DESCRIPTION OF THE DRAWINGS

So that the manner in which the features of the invention can be understood, a detailed description of the invention may be had by reference to certain embodiments, some of which are illustrated in the accompanying drawings. It is to be noted, however, that the drawings illustrate only certain embodiments of this invention and are therefore not to be considered limiting of its scope, for the scope of the invention encompasses other equally effective embodiments. The drawings are not necessarily to scale, emphasis generally being placed upon illustrating the features of certain embodiments of the invention. In the drawings, like numerals are used to indicate like parts throughout the various views. Thus, for further understanding of the invention, reference can be made to the following detailed description, read in connection with the drawings in which:

FIG. 1 is a profile view of a nano-droplet array plate (nDAP);

FIG. 2A is a profile view of a nDAP with a single geometric angle;

FIG. 2B is a profile view of a convex stem with a re-entrant angle;

FIG. 3 is a flow diagram depicting one method of forming an nDAP;

FIG. 4 depicts profile views of several shapes of nDAPs;

FIG. 5 is a flow diagram of another method of forming an nDAP;

FIG. 6 is a profile view of a lid with posts integrated therewith;

FIG. 7 is a profile view the lid with the posts pressed against a heated substrate;

FIG. 8 is a profile view of an nDAP resulting from the steps depicted in FIG. 6 and FIG. 7;

FIG. 9 and FIG. 10 is a front view and a side view, respectively, of a mold for forming an nDAP;

FIG. 11 and FIG. 12 is a front view and a side view, respectively, of an nDAP formed from the mold of FIG. 9 and FIG. 10;

FIG. 13 is a top view of the mold of FIGS. 9 and 10 in a closed configuration;

FIG. 14 is a top view of the mold of FIGS. 9 and 10 in an open configuration;

FIG. 15 is a front view of a different mold for forming an nDAP;

FIG. 16 is a front view of an nDAP formed from the mold of FIG. 15;

FIG. 17 is a profile view of a hot press system useful for forming an nDAP;

FIG. 18 is a flow diagram for one method for forming an nDAP with an hour-glass shape;

FIG. 19 is a flow diagram for one method for forming an nDAP with a cylindrical shape;

FIG. 20 is a flow diagram for one method for forming an nDAP with a conicial frustum shape;

FIG. 21 is a flow diagram depicting a method of forming an nDAP with anvil surfaces using a mold;

FIG. 22 is a flow diagram depicting a method of forming an nDAP with an hourglass shape using a mold;

FIG. 23 is a flow diagram depicting another method of forming an nDAP with anvil surfaces using a mold;

FIG. 24 is a flow diagram depicting another method of forming an nDAP with anvil surfaces using a mold;

FIG. 25 is a flow diagram depicting a printing-based method for forming an nDAP;

FIG. 26 is a flow diagram depicting another printing-based method for forming an nDAP;

FIG. 27 is a flow diagram depicting a method for grafting biotin and PEG to a surface;

FIG. 28 is a flow diagram depicting a method for grafting PEG to a surface;

FIG. 29 is a flow diagram depicting a method for grafting DMDCS to a surface;

FIG. 30 is a flow diagram depicting a printing-based method for forming an nDAP;

FIG. 31 depicts graphs showing consistent volumes of liquid being dispensed using an nDAP;

FIG. 32 is a graph depicting volume control and precision;

FIG. 33 is a flow diagram depicting a method for manual dispensing of nanoliter droplets;

FIG. 34 is a flow diagram depicting a method for using a dispensing rod for the transfer of a liquid;

FIG. 35 is a profile of several uses of the nDAP transfer methods illustrating good reproducibility when properly used;

FIG. 36 is a flow diagram showing transfer of a solution to a microplate;

FIG. 37 is a flow diagram showing transfer of a droplet to a solution using the solution's meniscus;

FIG. 38 is a flow diagram showing transfer of a droplet to a container;

FIG. 39 is a profile view of a nDAP system for transferring temperature-sensitive gels;

FIG. 40 is a profile view of a large volume of solution dispensed atop multiple nDAP surfaces;

FIG. 41A is a profile view of a system wherein an nDAP is enclosed within a chamber for high resolution imaging;

FIG. 41B is a CLSM image using the system of FIG. 41A;

FIG. 42A and FIG. 42B are profile views of polymer holders with a rigid window and a flexible window, respectively;

FIG. 42C and FIG. 42D are profile views of embodiment that creates a seal between the frame and the base is by using a PDMS base with a groove and gasket molded;

FIG. 43 is a flow diagram showing cells immobilized in a gel disposed on adjacent anvils;

FIG. 44 is a profile view of an nDAP array formed on a lid;

FIG. 45 is a profile view of an nDAP system similar to FIG. 44 except a thin spacer is used to place cells proximate a transparent then film bottom that permits imaging;

FIG. 46 is a profile view of an nDAP system similar to FIG. 44 that is used as a microwell plate system to grow tissues;

FIG. 47 is a profile view of an nDAP system used a microwell for growing special types of cells;

FIG. 48 is a profile view of an nDAP system used a microwell with microchannels;

FIG. 49 is a top view of several configurations of microchannels;

FIG. 50A is a profile view of a microwell with short posts for cell anchoring and long posts for lid support;

FIG. 50B depicts the microwell of FIG. 50A with cells disposed on the short posts;

FIG. 51 depicts the microwell of FIG. 50B with a medium disposed therein;

FIG. 52A depicts a microwell with a semi-permeable bottom;

FIG. 52B and FIG. 52C depict closed microwells in an upright and inverted configuration, respectively;

FIG. 53A illustrates a microwell for preparing multi-layer cell cultures;

FIG. 53B, FIG. 53C, FIG. 53D and FIG. 53E are schematic depictions of a method for sequentially depositing different cell types to form a multi-layer cell culture;

FIG. 54 is a flow diagram depicting a method of using cells immobilized within a hydrogel;

FIG. 55 is a flow diagram depicting a method to encapsulate cells in a hydrogel;

FIG. 56A is a flow diagram depicting method for forming multiple shells around a core;

FIG. 56B is a profile view showing a single anvil with a multi-shelled core;

FIG. 57 is a flow diagram depicting a method for dissolving a core to form a liquid core-shell hydrogel drop;

FIG. 58A depicts a method for forming cell spheroids with specific sizes;

FIG. 58B depicts a method for anchoring cells to a well;

FIG. 59 depicts a method of opening a core-shell drop to retrieve cells that have been developed;

FIG. 60 is a magnified view of a collagen core with a shell structure encapsulating calcium carbonate particles;

FIG. 61 is a magnified view of a 2 μL droplet manually transferred to a glass pedestal;

FIG. 62 depicts a graph showing dispensed volume and contact angle as a function of lift velocity with images of the droplets also shown;

FIG. 63 depicts a graph showing dispensed volume and contact angle as a function of source droplet volume; and

FIG. 64A is a graph depicting fluorescence intensity increasing with increasing concentration of NeutAvidin while FIG. 64B shows the same with increasing mass of NeutAvidin.

### DETAILED DESCRIPTION OF THE INVENTION

This disclosure pertains to nano-droplet array plates (nDAPs). The disclosure provides an economically feasible way to produce nano-drop array plates that provides micro/nanoliter volumes of fluids with unprecedented precision. The nDAPs can be used as a single isolated dispenser or as an array of receptacles, or as an array of droplet dispensers, or as an array of droplet receptacles (e.g. a nanoliter droplet virtual well microplate).

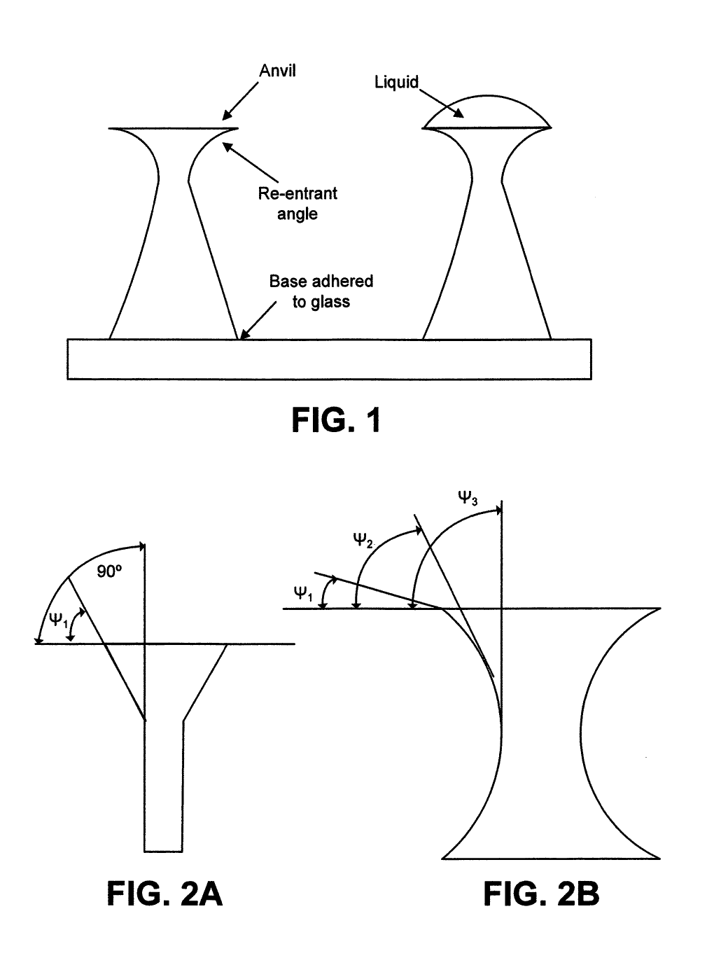

Referring to FIG. 1, the examples disclosed herein have flat tops that come to a sharp, re-entrant angle with a sharp point and continuous curve running from the sharp point to the apex (i.e. where the two cones meet). The steepness of the actual curve may vary. This curved “hour glass” shape is a characteristic feature of at least some embodiments. In the embodiment of FIG. 1, the top diameter of the flat top is less than the bottom diameter of the post and the middle section has a diameter that is less than both the top diameter and the bottom diameter.

In one embodiment, the nDAP has a convex stem as is shown in FIG. 2B. The nDAPs have a concave curvature under the top, solid surface. nDAP has a continuously varying geometric angle ψ that begins at about 0° and increases to 90°. This angle defines the concave curvature. In the embodiment of FIG. 2B, the upper half has a continuously varying geometric angle (ψ) that begins at 0° and increases to 90°. In the embodiment of FIG. 2A at least one geometric angle (ψ) is present that is greater than 0° and less than or equal to 90° degrees. In FIG. 2A the middle section of the post has a diameter that is less than the top diameter but greater than the bottom diameter.

In one embodiment, the nDAP is formed from (consists essentially of) only one material and are monolithic. The material may be PDMS (Polydimethylsiloxane) or a thermoplastic such as polystyrene, polyolefin, polycarbonate or acrylonitrile butadiene styrene (ABS). In some embodiments, the nDAP provides a superhydrophobic surface without requiring surface treatment or other post-fabrication treatment. In other embodiments, the surface of the nDAP is modified to render it hydrophilic.

In one embodiment, the nDAP provides isolated structures with individual, isolated re-entrant surface features such that a single drop is supported on a corresponding single surface. An array of these features is formed, with a separation distance between features of D, not so that they can act in a cooperative manner, but so each individual feature can hold an individual droplet. The nDAP surface features are grouped into arrays for the convenience of the user—and not to modify the properties of the surface. Below a certain value of D, droplets may span across two features, reducing the precision of the nDAP when used as a dispenser or as a receiver of droplets. Above a certain value of D, the advantage of reducing the distance between features is that more features may be packed together per square area, increasing the usefulness of the device for certain applications—however reducing this distance does not improve the wetting properties of a feature. Specifically, because the gap between features for the disclosed nDAP lid surfaces have a capillary length much greater than, or close to the capillary length of the fluid of interest (water), the nDAP lid surfaces do not permit bridging of two adjacent nDAPS by a single drop of fluid.

Advantageously, the method of manufacture does not rely on the use of expensive lithographic techniques and, as such, renders the production of relatively large arrays economically feasible.

### Example 1

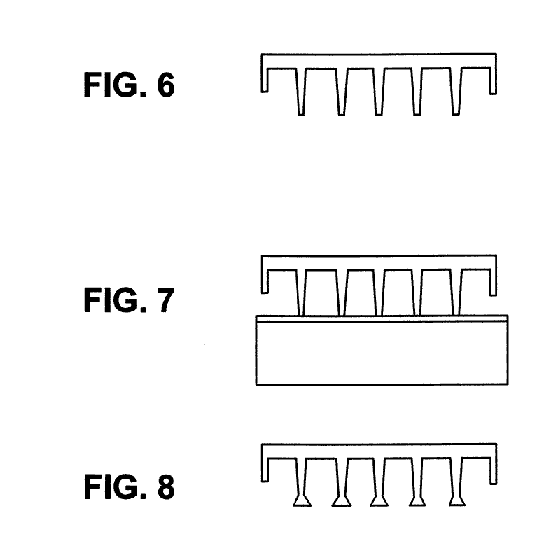

Acrylonitrile butadiene styrene (ABS) rod from 3Doodler was sharpened using a manual pencil sharpener. A hot plate was preheated to 150° C. The ABS rod was aligned perpendicular to the hot plate surface with the assistance of a rod holder and a position holder as shown in FIG. 6. A polyimide sheet with a thickness of 10 mil was placed on the hot plate and used as releasing layer. The tip of the ABS rod was then brought into contact with the hot surface. The tip in contact with the hot plate became soft and the polymer flowed along the surface of the hot plate under the gravity of the rod and the rod holder. By controlling the contact time, the diameter of the tip can be well-controlled. For example an nDAP tip with a diameter of 1000 μm was achieved when the contact time was 10 seconds. The polyimide sheet was lifted off together with the rod after this step and separated from the tip when the ABS cooled and solidified

### Example 2

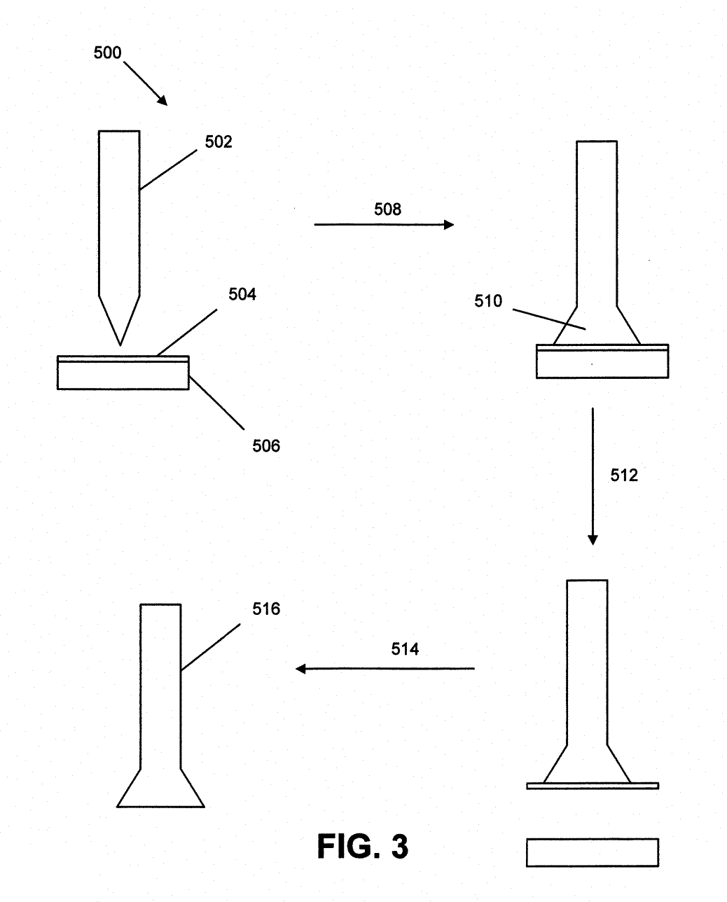

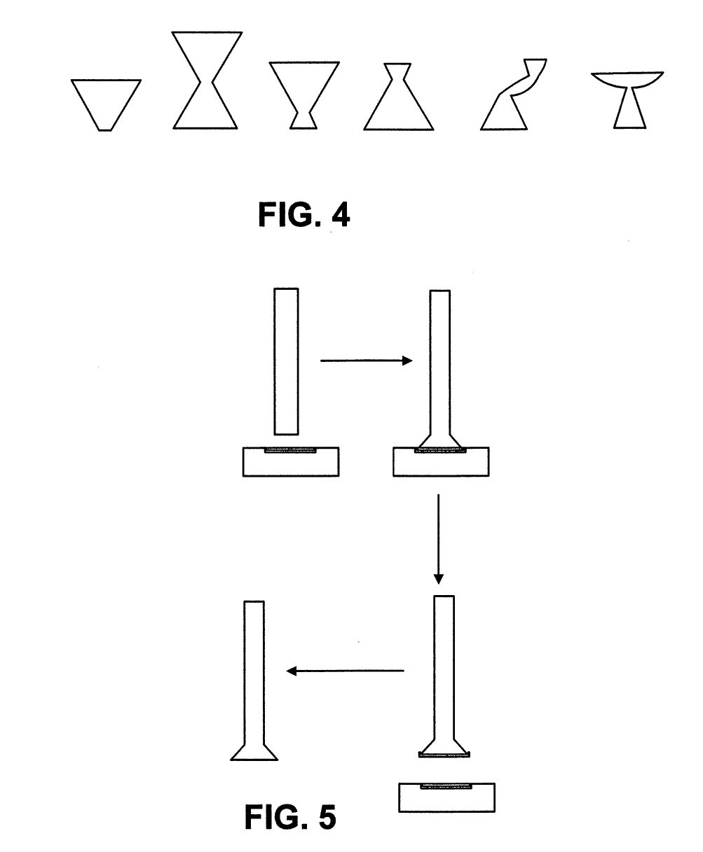

PLA (Polylactic acid) rod from 3Doodler was sharpened using a manual pencil sharpener. A hot plate was preheated to 150° C. The PLA rod was aligned perpendicular to the hot plate surface with the same set-up and same releasing layer as shown in FIG. 3. The diameter of the formed nDAP tip was 1000 μm when the contact time was about 2 seconds, and about 2900 μm when the contact time was 10 sec.

As shown in FIG. 4, the final shape of the nDAP posts are determined by a combination of the printing, pressing and stretching parameters. The cross-sectional or profile view of an nDAP post could be: a trapezoidal shape (with little or no stretching), an hourglass shape (with large stretching), or an asymmetric hourglass shape (with intermediate stretching) as shown in the first four images of FIG. 4. Many other, more complicated shapes can also be formed, a few of which are shown schematically in the last two images of FIG. 4. The shape profile can be adjusted by translating or rotating the top plate with respect to the bottom plate during the stretching and curing steps. These sloped or asymmetric shapes may prove beneficial for dispensing specific volumes of fluids, or to facilitate the direct contact of adjacent cell-in-gel deposits for the study of cell-to-cell communication.

FIG. 5 depicts a method that combines the hot press with the molding method. The steps are similar to the hot press method described elsewhere in this specification, except that a disk-shaped cavity is built into the surface of the hot plate. The center area of the bottom of the mold could be flat and the edge of the mold could be curved for better releasing properties. This cavity mold could help improve the uniformity of the size and curvature of the fabricated nDAP tips. The first step is to prepare a polymer rod and align the center of the tip to the center of the mold. The second step is to lower the polymer rod to contact the mold which is heated above the softening temperature of the polymer rod. In this step the softened polymer can flow and fill the mold under gravity or a certain pressure to accelerate the processing. The third step is to lift the polymer to remove it from the mold and let it cool in air. The last step is to remove the release layer. Single nDAP tips, 1D arrays and 2D arrays of nDAP tips can be fabricated using the hot press combined with molding method. Various polymer materials including both thermoplastic and thermoset materials that have a rigid shape can be used to make nDAP tips by these two methods. Releasing materials used for all methods could be a coating, a layer of oil, a thin sheet of polymer, or other materials.

Device: nDAP Lid for Microplates

3D tissue specimens, cultured in-vitro, holds great potential to provide an improved model for drug discovery over traditional 2D-cell culture. Moreover, these 3D cell agglomerates and micro-tissues may reduce or even eliminate the use of animal models and human subjects for drug research and discovery. Various scaffold-based or scaffold-free technologies have been developed to grow vitro 3D tissues. These technologies are often combined with standard microplates to grow microtissue arrays for high throughput screening applications. Examples of commercially available 3D culture platforms or “ware” include micro/nano-patterned microplates (e.g. Cytoo), hanging drop microplates (e.g. InSpherio), and microplates with ultra-low attachment coating (e.g. Corning). These current microplate based systems could meet the basic requirement for growing 3D microtissues. However, the procedures for working with these wares are cumbersome and there is a high risk of damaging the microtissues during handling. Especially challenging steps include: removing and adding medium or other reagents while the fragile microtissues are in the well; transferring the microtissues to other plates for culturing; and characterizing the microtissues by imaging or other techniques. These steps are difficult to perform by trained researchers and are essentially impossible to automate using commercially available laboratory robotic systems.

For some types of tissue, such as skin, it is important to expose the outer surface of the microtissue to air so that the cells differentiate appropriately. Commercially available ware/plates, including those described above, cannot produce a liquid-air interface under the high humidity conditions required to grow skin tissues. A novel microplate system that can eliminate these cumbersome procedures, form the liquid-air interface easily and enable the imaging of the 3D microtissues in the culturing plate upon request are highly desirable for many applications in the pharmaceutical industry, toxicity screening labs, cosmetic industry as well as advanced academic research.

Multi-well microplates are used extensively by researchers and large pharmaceutical companies for a wide variety of functions including: culturing cells, conducting diagnostic tests (e.g. ELISA tests), drug screening, etc. For some applications, it can be advantageous to include a compound or cells within a gel (e.g. hydrogel) within the well of a standard microplate. The gel should immobilize the molecule or cells but otherwise not interfere with the function of the microplate test. Thus if an nDAP could be formed in each well, a precisely defined volume of solution that contains molecules or cells can be delivered. If the solution also contains a gel, then the molecules and/or cells can be adhered onto the nDAP surface such that small molecules can diffuse into or out of the gel and interact with the solution in the well. There are several ways in which the nDAP can be incorporated or used.

In one implementation, an nDAP can be formed at the bottom of each well of an otherwise standard microplate. This has the advantage that the drop dispensed on the nDAP can be maintained in solution (e.g. cell culture medium). To fabricate such a structure, PDMS can be dispensed into the bottom of the well and then formed into the anvil-like nDAP shape as described elsewhere in this specification. In some embodiments, a UV curable or room-temperature curable resin is used as a thermal cure could disrupt the dimensions of the microplate. Alternatively the nDAP can be pre-formed and adhesively bonded to the bottom of the well.

One disadvantage of nDAPs at the bottom of the well is that for some functions, the nDAP can obstruct measurements of the wells. For example, it may be difficult to measure the UV-vis transmission or fluorescence, chemiluminescence, etc. from a well with an nDAP bonded to the bottom. A second disadvantage is that it may be difficult for a vision controlled dispensing system to see the top of the nDAP and align the incoming droplet to the nDAP within the well.

In one embodiment, the nDAP array is formed on the lid of a standard microplate. This approach has several advantages over nDAPs formed at the bottom of the wells. As described elsewhere in this specification, PDMS or other polymers can be printed onto the lid to form a cone-shape. The top of the cone is then flattened and cured. A lid composed of 96, 384, 1536 or more print deposits can be planarized and cured simultaneously. Thermally cured materials can be used, but care should be taken such that the heat required to effect cure does not adversely affect the mechanical shape of the lid. UV-cured and room-temperature cured materials may be advantageous. Alternatively, the cone-shaped nDAP precursor can be formed from thermoplastic polymers. Either the thermoplastic polymer can be printed onto the pre-molded lid (e.g. using a 3D printer), or the lid can be molded into a near-net-shape with the cones, or elongated posts, integrated into the lid as shown in FIG. 6. Regardless of the means of deposition, the cones are subsequently “anvilized”, i.e. the thermoplastic cones are pressed against a heated substrate using any appropriate method (e.g. methods described in section 1 of this document), as shown in FIG. 7, to form cones (or cylinders, or elongated posts) with flat tops and sharp edges that exhibit reentrant angles as shown in FIG. 8. In one embodiment molding a cover with integrated posts is provided with the posts present in an array (e.g. an 8×12 grid of posts). The length of the elongated posts can be controlled so that the nDAP tip surface will be above the bottom of the well once the lid is in place. The height above the well bottom can be controlled depending upon the size/volume of the drops and the requirements for the particular function. By designing the structures so that the nDAP surfaces are as near the bottom of the well as possible, smaller volumes of expensive/precious/rare reagents will be required which would be advantageous for lowering overall cost. The dimensions of microplates are standardized by the Society of Laboratory Automation and Screening (SLAS) to provide a predictable configuration. Each fabricated structure may be spaced from adjacent structures to coincide with one of these standardized microplate configurations (e.g. spaced apart to match a 96, 384 or 1536 well configuration).

This disclosure provides several fabrication methods. These fabrication methods include: (1) a compression/injection molding method, (2) a hot press method and (3) a combination hot press with molding method. These methods can fabricate a single nano-droplet array plate (nDAP), a row of nDAPs or a two-dimensional array of nDAP tips. Each method provides an nDAP with a top planar surface that connects to an elongated rod with a re-entrant angle.

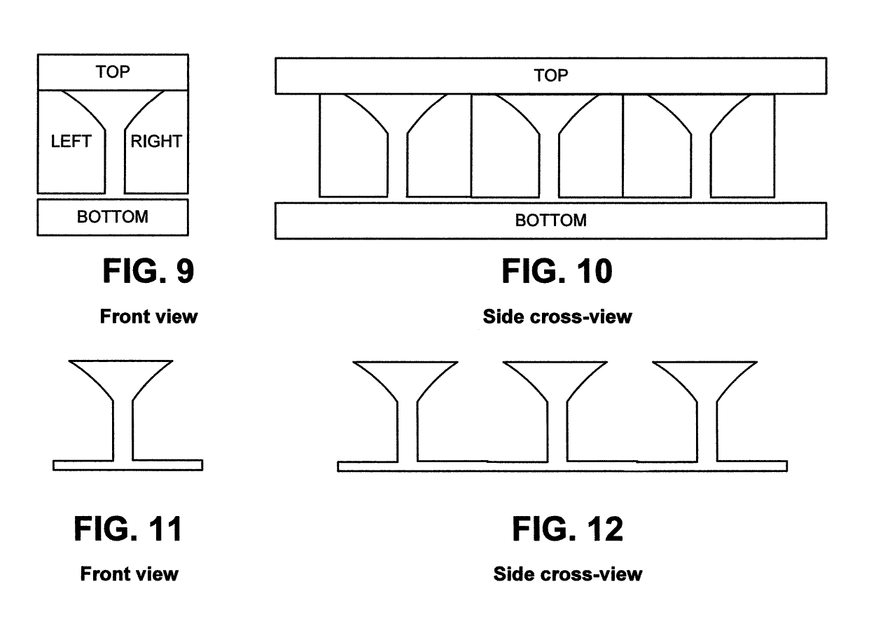

FIGS. 9 to 12 depict a compression and/or injection molding method for fabricating nDAP tips that offers a low-cost route to fabricating these useful shapes. The mold can contain four basic components as shown in FIG. 9 and FIG. 10 that results in a row of connected nDAP parts shown in FIG. 11 and FIG. 12. FIG. 9 illustrates a left component and a right component, each having a semi-conical shape. These components can be put together to form a whole conical shape cavity. The top and the bottom components are used to close the cavity from top and bottom respectively.

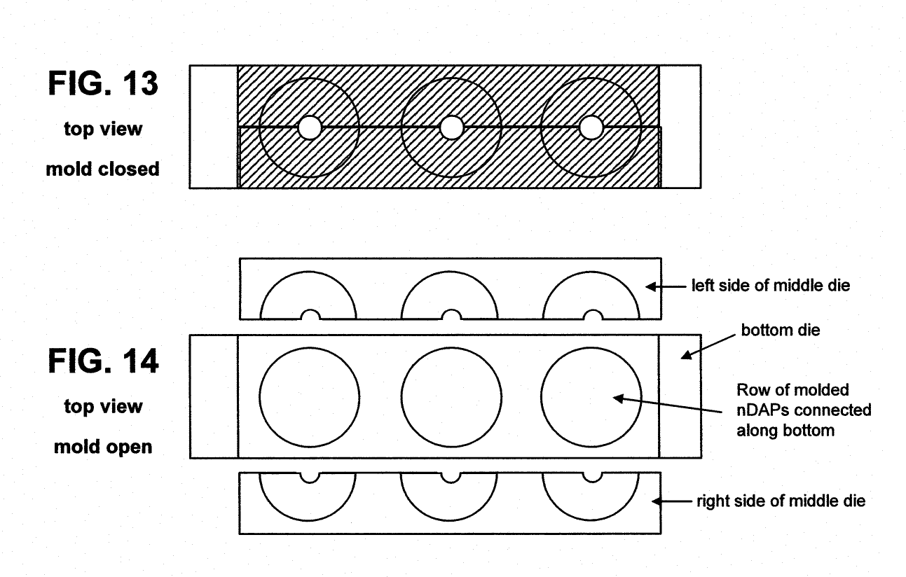

An illustration of the mold as seen from the top, is shown in FIG. 13. Single nDAP tips or a row of interconnected nDAP tips can be fabricated using such molding methods. After the polymer (or other material) hardens, the mold is opened and the middle sections of the mold slides outwards to release the finished parts. FIG. 14 depicts the mold in an open configuration.

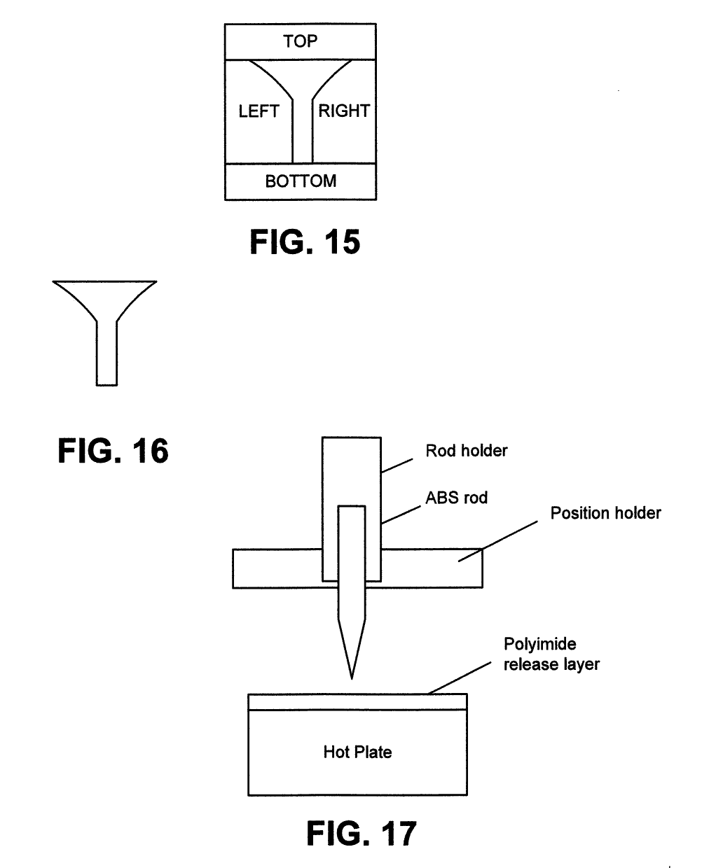

Individual nDAP tips can be formed separately if the bottom component of the mold is fully closed or formed into an integrated piece. A two-part mold and molded part is shown in FIG. 15 and FIG. 16, respectively. An array of such parts may easily be made; runners may connect the individual parts. The key aspect of such molding approaches is to design the mold such that the parts can be removed after the polymer (or other material) solidifies and the sharp, re-entrant perimeter of the anvil-shaped top remain crisp and sharp.

Materials in liquid or semisolid form can be filled into the mold under pressure with or without the assistance of vacuum. Release coatings, oil or other materials can be applied onto the inner surface of the mold if necessary. A broad range of materials can be used with this molding method to make the nDAP tips, including various thermoplastic polymers, thermoset polymers, various precursor solutions, composite resins and melted metals and glasses.

As shown in FIG. 2, a hot press method 500 has been developed for making the nDAP tip. A polymer rod 502 is aligned perpendicular to a surface of a hot plate 506. Also see FIG. 17. In some embodiments, to make the smallest diameter nDAP tips, a relatively larger diameter rod is sharpened at the tip to make a point. The temperature of the hot plate 506 should be above the softening temperature (e.g. Tg (glass transition temperature), or Tₘ (crystalline melting point), or B-stage temperature) of the polymer rod. In step 508 the polymer rod 502 is lowered to bring the tip into contact with the hot plate 506. Pressure can be applied to accelerate the flow of the thermoplastic or thermoset polymer along the hot plate surface. In this step a disk shape is formed at the interface between the rod and the hot plate. In one embodiment a release layer 504 is placed on the hot plate surface. The releasing layer 504 may be used to prevent adhesion of the polymer rod 502 to the hot plate 506. Upon warming, an anvil shape 510 is formed as the polymer flows. In step 512 the anvil shape 510 is separated from the hot plate 506. In step 512, the releasing layer 504 is separated from the anvil shape 510 to product an nDAP 516. These two steps can performed simultaneously in the case when the releasing layer is not needed or is bonded onto the hot plate. In this case, the tips are cooled with the hot plate when a thermoplastic material is used. FIG. 3 shows the case when a thermoplastic polymer rod is used and a releasing layer is placed on, but not adhered to the hot plate surface. The release layer can be lifted off with the polymer rod in step 514, and be separated from the nDAP tip after the polymer is cooled and solidified. Both single nDAP tips and 2D arrays of nDAP tips can be fabricated using this method.

As discussed previously, the volume of droplets can be reduced from nanoliters to picoliters by reducing the diameter of the nDAP anvils. A post with a diameter of 100 μm can generate droplets of 130 picoliters. Evaporation of picoliter volumes is one of the main challenges for handling miniaturized droplets. A high throughput parallel dispensing process has been developed that can generate arrays of nano/picoliter droplets at high throughput by a simple touch and lift-off procedure. An array of 496 posts with a tip diameter of 150 μm was dipped into a well containing an alginate solution so that the anvils contacted the surface of this source solution. As the nDAP was raised, droplets form on the anvils with a volume of 0.59 nanoliters. The array of gel droplets was then immersed into another well containing calcium chloride solution to cure the gel. After curing, the gel droplets are firmly anchored onto the nDAP. This adhesion stability has allowed us to perform many subsequent manipulations such as cell stimulation, chemical treatment, washing, as well as changing the medium solution for long term cell culture studies without disturbing gel droplet anchoring. The parallel dispensing method reduces the exposure time to air such that picoliter droplets can be manipulated in an 85% RH environment.

The geometry of the anvil surface, produced by the two methods described above, is described by a circle. Other anvil shapes are possible if the release layer is formed into a pattern. In addition to circles, other shapes including: triangles, rectangles, heart shapes or other regular or irregular shapes can be formed by generating the corresponding protrusion patterns in the releasing layer. As the edges of the thixotropic resin (e.g. PDMS) flow along the release layer, the flow would be limited by the edges of the protrusive patterns during the pressing step or the printing step. As a result, the leading edge of the flow would be stopped by the specified pattern. The pattern shape would be preserved during the stretching and curing steps. Different anvil shapes could prove beneficial for various aspects of nDAP use, including anchoring of gel onto the anvil, controlling the volume of fluid dispensed, and/or imaging of cells immobilized on the nDAP surface.

The ability to dispense fluids on the anvil surface can be enhanced by treating the top surface of the nDAP to be hydrophilic by various methods, such as dry plasma treatment, wet chemical etching methods, or other surface coating technologies. Chemically modifying the top of the anvil surfaces facilitates dispensing fluids because high surface energy liquids, like water, will bind more strongly to the hydrophilic surface treatment and not easily roll-off the nDAP surface.

A second advantage to chemical modification of anvil top surfaces is that each anvil can become the site of an immunoassay, such as an enzyme-linked immunosorbent assay (ELISA) or a fluorescence-linked immunosorbent assay (FLISA). The chemically modified surface of the polymer (e.g. modified PDMS) can react with groups that either bind antibodies, antigens, or moieties that can bind with proteins such as biotin. For example, an oxidized PDMS surface can react with molecules that contain a silane group. The oxidized PDMS surface will then be modified such that the other portion of the molecule is exposed to the solution. For example, if the silane is attached to biotin (e.g. PG2-BNSL-600, Silane-PEG-Biotin, 600 Da purchased from Nanocs), then the anvil surface can be coated with biotin. This biotinylated surface can then selectively bind to antibodies or antigens linked to an avidin protein (e.g. NeutrAvidin). In this way, specific antibodies or antigens can be selectively bound to the anvil surface. Once bound, these antibodies or antigens can be used in standard immunoassays such as ELISA or FLISA tests.

In addition to reacting with silanes, the hydrophilic groups formed on the surface could also react with a wide variety of biomolecules (e.g. proteins, peptides, etc). Any of these surface groups not covalently bound to a target molecule (e.g. through a silane linkage) would need to be treated to avoid the non-specific binding of adventitious biomolecules. There are several approaches that have been described in the literature to prevent the non-specific binding of biomolecules; two examples are given here. In the first example, the surface is exposed to an excess of a protein that will not affect the test. Often bovine serum albumin (BSA) is used as it is readily available. A second approach is to bind a polyethylene glycol silane (PEG-Silane) such as PG1-SL-550 from Nanocs or PSB-2017 from Creative Pegworks. The PEG groups chemically bound to the anvil surface are well known to prevent protein and other biomolecule adsorption on surfaces.

In this way the treated anvil post surface is composed of groups that selective bind a specific biomolecule and these sites are surrounded by groups (either chemically bound or adsorbed) that resist biomolecule adsorption. A droplet containing the test solution can then be dispensed on the surface and the corresponding test conducted. The advantage of this nDAP type of immunoassay, compared to conventional immunoassays, is that small (picoliter to nanoliter) volumes of test solution can be used thus reducing the quantities of precious and expensive bioreagents needed. This is especially important for testing rare natural products such as venoms isolated from spiders and snails. In addition, the test results can be acquired directly on the nDAP surface using high-content screening techniques such as fluorescence microscopy.

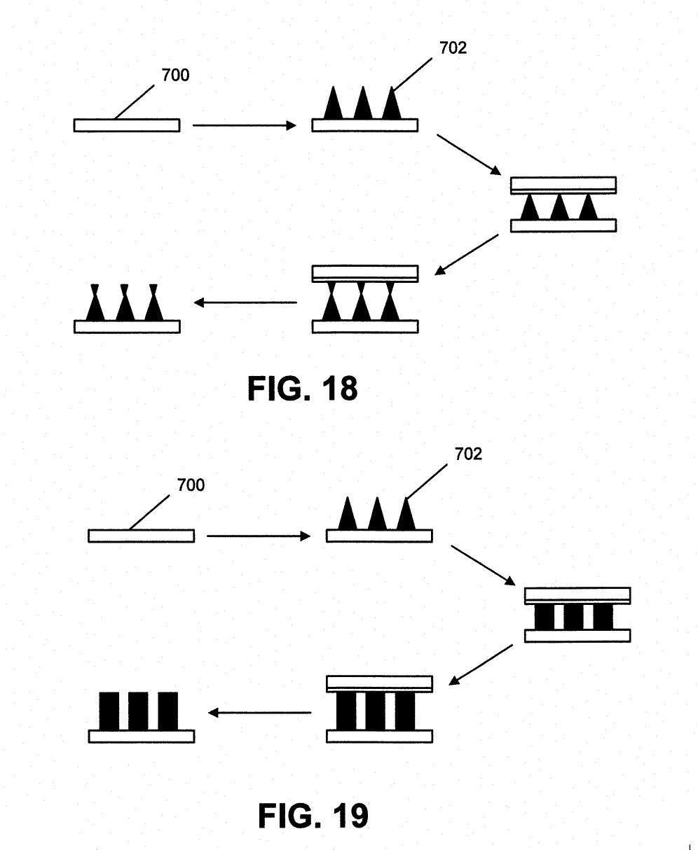

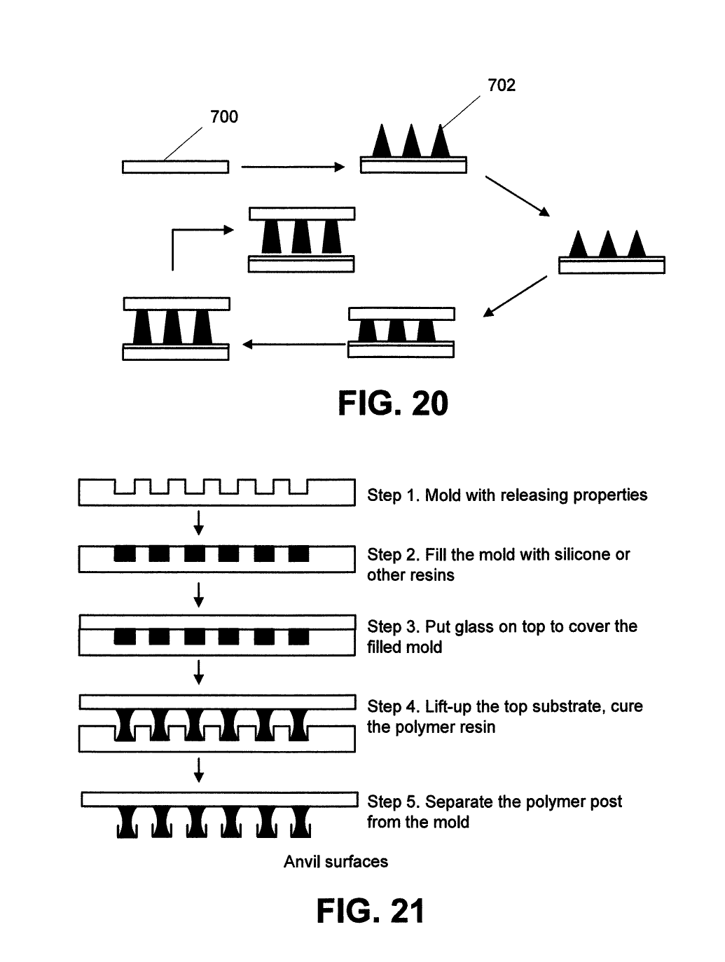

FIG. 18 and FIG. 19 depict two hot press methods. A glass substrate 700 is provided and a polymeric resin 702 (e.g. a polydimethylsiloxane (PDMS) resin) is printed to a surface on the glass substrate 700. A hot plate with a release layer is pressed to the polymeric resin (thereby compressing the resin) and subsequently stretched and cured. After curing, the plate is removed to reveal a planar top surface. The elongated post may be hour-glass shaped (FIG. 18); cylindrical (FIG. 19); conical frustum (FIG. 20); or other similar shape. In one embodiment, the resins are printed onto a substrate using a three-dimensional (3D) printer.

In the embodiment of FIG. 20 the polymeric resin is printed into a release layer and subsequently compressed with a substrate to form cylinders. The cylinders are stretched and cured while in the stretched position. The cylinders are subsequently released from the release layer and remain attached to the substrate forming inverted conical frustum posts.

Microwell Molding and Stretching method A: This method is based on a molding method as shown in FIG. 21. The microwell mold is treated with a releasing layer or directly made of a low-energy material such as TEFLON®. Polymer resins with sufficient high viscosity or thixotropic properties are filled into the mold. A flat substrate such as glass is put onto the filled mold under a certain pressure so that the resin in the well are in contact with the glass. Then the substrate is lifted up to a certain height, and the polymer resin is cured in position. After cure, the polymer posts are removed from the mold to expose the “T” microstructures. Such a surface could be extremely superoleophobic because of the extreme re-entrant angle.

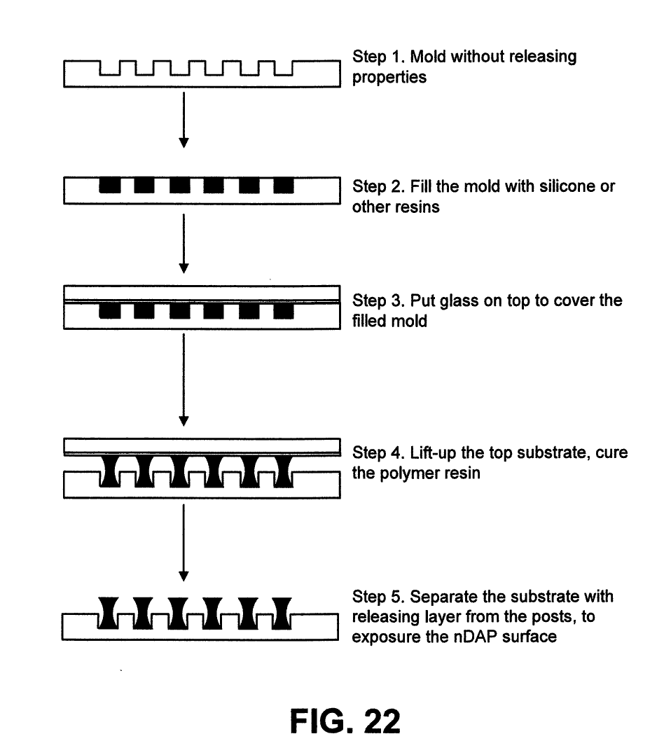

Microwell Molding and Stretching method B: This method is similar to the prior method except that the releasing layer is applied on the flat substrate instead of the wells. As shown in FIG. 22, polymer resin is filled into the microwell mold without releasing properties, and the flat substrate treated with a releasing layer is put onto the filled mold under a certain pressure so that the resin in the well can contact with the top substrate. Then the substrate is lifted up to stretch the polymer resin to a certain height and the polymer resin is cured in position. After cure, the polymer posts are separated from the releasing layer to expose the nDAP surfaces.

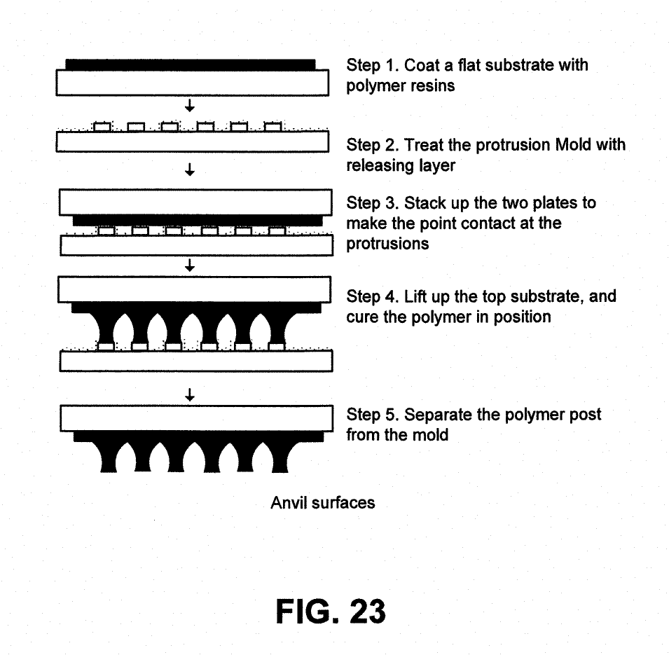

Protrusion molding and Stretching: Protrusion molding (using a mold with structures protruding from the surface) also can be used to make nDAPs. A schematic of this method is shown in FIG. 23. At first a flat substrate is coated with a layer of the polymer resin, and a protrusion mold is either coated with a release coatings or made directly from a releasing material such as TEFLON®. Then the two substrates are stacked up to make contact between the resin and the protrusions. The two substrates are moved away from each other to stretch the resin layer to a certain distance to form an “hour-glass” shape and cured in place. After curing, the polymer posts are separated from the protrusion mold to expose the nDAP surfaces.

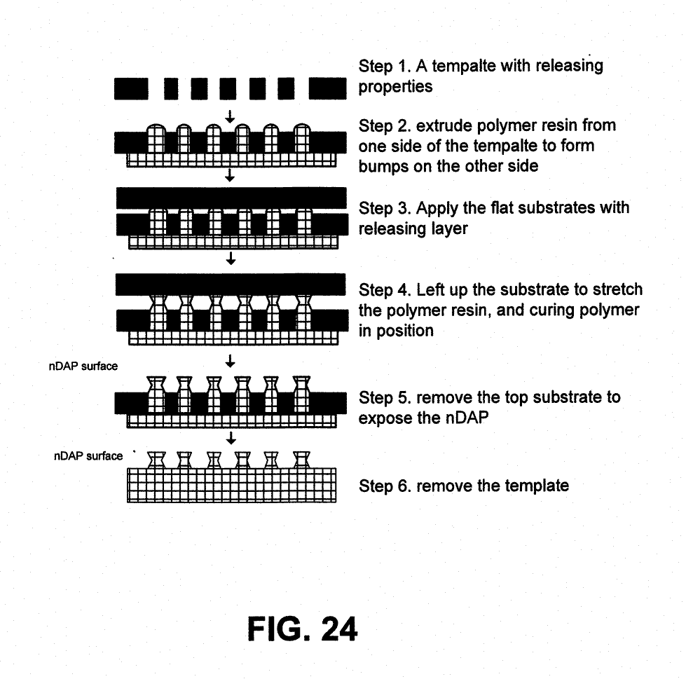

Extrusion Molding and Stretching: The extrusion method involves a template with through holes, such as a woven mesh, to make nDAPs. A schematic of this method is shown in FIG. 24. The template is treated with a release coating or made directly from materials with releasing properties such as TEFLON® materials. Polymer resin is extruded from one side of the template under a certain pressure to form bumps on the other side of the template. Extrusion can be done at room temperature (e.g. when using PDMS resins) or at elevated temperatures (e.g. when using thermoplastics). A flat substrate with a release coating is placed in contact with the formed bumps on the template and lifted up to stretch the resin to form an hour-glass shape. The polymer resin is cured in position. After cure, the polymer posts are separated from the top substrates as well as the extrusion mold to expose the nDAP surfaces. As the template could be made from a low-cost fabric mesh, the template can be left with the polymer resin to improve the mechanical properties of the polymer substrate. In this case, the release coating is not necessary for the template.

To ensure that all anvils have the same diameter, both within one nDAP array, as well as between different nDAPs of nominally the same type, an automated nDAP manufacturing process could be used. In this process, a computer imaging system is used to measure the diameter of the anvil during the pressing step until a pre-determined diameter is recorded. As soon as the correct size is detected, the nDAP is stretched until the correct height is detected using either an optical imaging system, or through sufficient control of the robotic assembly station. Once the nDAP is stretched to the correct amount, heat is applied, either conducted through the top and bottom substrate, or by IR/UV/microwave illumination. Molding of nDAPs with these features is also possible.

Other heat curing, UV curing, and thermoset materials could also be used to print the conical features that are pressed, stretched and cured to make nDAPs. Theoretically any polymer resin or resin composites with a sufficient high viscosity and/or thixotropy can be used in either method. Either hydrophilic or hydrophobic resins could be used. For the printing to work well, the rheological properties of the resin, such as high viscosity and/or shear thinning behavior are preferred.

The release layer can be made from a low surface energy polymer or a nonstick substrate. Examples include: TEFLON®, polyethylene, polyethylene terephthalate (PET) or other films. TEFLON® films such as FEP, PFA, PTFE and ETFE film are the most preferred materials for the release layer. The films can be used as stand-alone films or bonded to a flat rigid surface (e.g. glass). Small molecules, such as fluorosilanes, could also be bonded to a rigid surface and used as a release layer, although the durability may not be as good and the fluorosilane treatment would need to be repeated periodically.

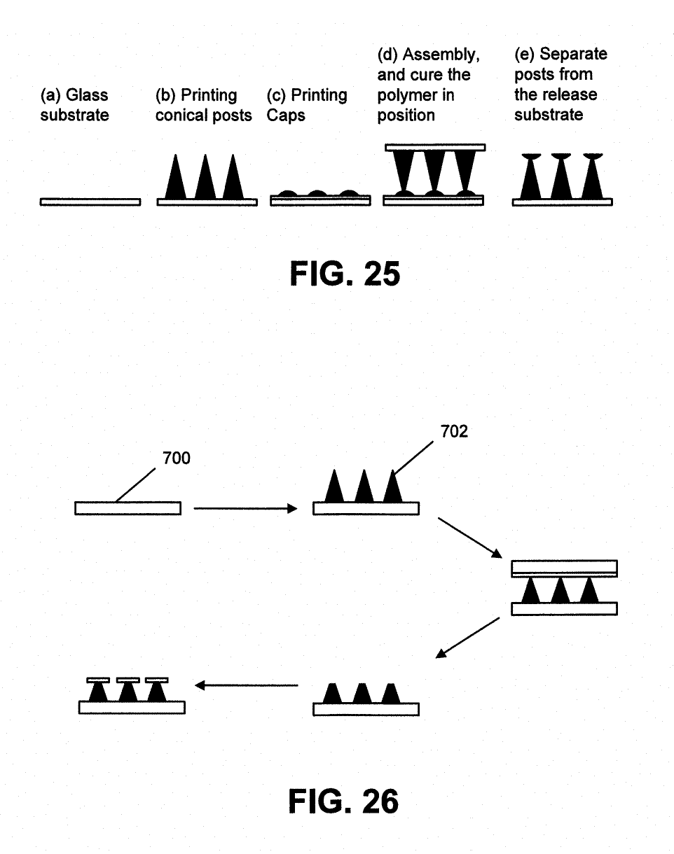

Printing conical posts and caps, and assembly: As shown in FIG. 25, conical posts of polymer resins with high viscosity or thixotropic properties are printed onto a substrate (e.g. glass) the same way as previously described. Drop-shaped dots with the same pitch are printed onto a flat surface coated with a releasing layer, such as TEFLON®. The two parts are aligned, assembled together and cured in position as shown in FIG. 25. After curing, the posts are separated from the substrate coated with a release layer to exposure the nDAP surface.

Alternatively, the conical posts and the caps could be either cured or uncured before the assembly. If both are cured, an adhesive layer will be needed for bonding the post and the caps together. The posts and the caps could be the same materials or different materials. If they are different materials, a specific adhesive layer may be needed to bond the two materials together.

The rigidness of the post as well as the caps can be adjusted by using materials or composites with different stiffness, or changing the aspect ratio between the height and the diameter of the base. Different from the method disclosed previously, the dimensions of the posts and the caps, such as diameter and height can be changed separately using this method.

A nanoliter droplet virtual well microplate (nVWP) for precisely dispensing nanodroplets on top of isolated pedestals is also provided. University researchers as well as large pharmaceutical companies need improved technologies for discovering new drugs. Current drug-discovery approaches are highly inefficient, as less than 10% of new drugs entering the pipeline receive FDA approval. The tools used today may explain this low success rate as researchers typically study the average response of a large number of cells and/or culture cells in a non-natural, 2D environment. Studying the response of individual cells as a function of genotype and phenotype when cultured in a more physiologically relevant 3D environment is expected to increase drug-discovery success rate. High precision (better than +/−2.2%) was achieved through the combination of local surface chemistry and the geometry of the pedestals, i.e. the sharp edge created at the glass-air interface. Such a device relieves the mechanical alignment tolerances required for dispensing compared to conventional dispensing techniques and provides a significantly improved method to accurately dispense and manipulate nanoliter droplets from source sample volumes as small as 5 μL. The wetting behavior of nanodroplets dispensed on the nVWP, and the position of the solid-liquid-vapor TCL interface, was studied using optical microscopy during dispensing as well as by determining the position of fluorescently labeled proteins deposited on the surface after droplet evaporation. The effects of glass surface chemistry and dispensing parameters (e.g. dispense tip lift-up velocity, source droplet volume) on dispensed volume were systematically studied. The high precision nVWP dispensing platform can be used for a variety of different assays including sensitive detection of proteins and peptides by both fluorescence microscopy as well as MALDI-TOF. In addition, the glass pedestal surface was functionalized to enable the selective adsorption of specific peptides/proteins from biomolecule mixtures. Biotin and KcsA ion channels were bound to PEGylated or nickel-chelate resin coated glass pedestals and these surfaces were shown to selective adsorb NeutrAvidin (from a mixture with BSA) or TX7335 peptide from snake venom.

In FIG. 26, another example method is depicted. An array of polydimethylsiloxane (PDMS) posts were printed onto a glass substrate using a robotic dispensing system (Janome 2203N and EFD Performus syringe dispenser). Briefly, PDMS silicone resin with thixotropic properties (ELASTOSIL® LR 3003 50A/50B, Wacker) was loaded and degassed in 10 cc syringes fitted with a 22 gauge tapered tip mounted to the robot. The robot is programmed to bring the tip to the first location at a controlled height of 2 mm above the glass microscope substrate. The robot triggers the syringe dispenser to deposit a controlled amount of PDMS (85 psi and 0.3 seconds) and then lifts vertically from the surface. This forms a single cylindrical cone of PDMS with a base diameter of 700 μm and a height of 1.2 mm. An array is created by repeating this procedure on 1.2 mm pitch. After printing, the conical posts were planarized during cure (165° C. for 5 min) by contacting the tips with a flat, TEFLON®-coated plate. After curing, the plate was easily released, exposing the tapered posts with flat tops. Glass pedestals measuring 500×500×100 μm (diced from glass coverslips by Valley Design Corp) with or without surface modification were mounted onto the flat tops using a room-temperature vulcanizing (RTV) hydroxyl-terminated dimethyl siloxane (DAP, Dow Corning) adhesive. Alternatively, the glass pedestals could be placed directly onto the PDMS cones before cure, using the accuracy of the placement/alignment machine to ensure the top glass pedestal surfaces are coplanar. The height of the PDMS cones are to provide additional compliance in the direction perpendicular to the substrate when dispensing fluids onto the glass pedestals. Thus the PDMS pedestals are not required to control droplet volume, but only help prevent the source droplet from inadvertently contacting the substrate.

Prior to mounting, glass pedestals may be functionalized. The glass pedestals were dismounted from the carrier substrates by soaking in acetone solution for 1 h in an ultrasonic bath (Fisher Scientific Inc.) to remove the dicing tape. The dismounted glass pedestals were rinsed twice in DI water and dried. The glass pieces were then thoroughly cleaned using the following procedure: 1) Washed in an aqueous solution of 4 wt % hydrogen peroxide and 4 wt % ammonium hydroxide at 80° C. for 10 min; 2) Rinsed with DI water three times; 3) Washed with an aqueous solution consisting of 4 wt % hydrogen peroxide and 0.4 M HCl at for 80° C. for 10 min.; 4) Rinsed with DI water three times; 5) Dried in an oven for 12 h at 60° C. The glass pieces were then ready to assemble onto the PDMS post arrays, or the surfaces were further modified as described below.

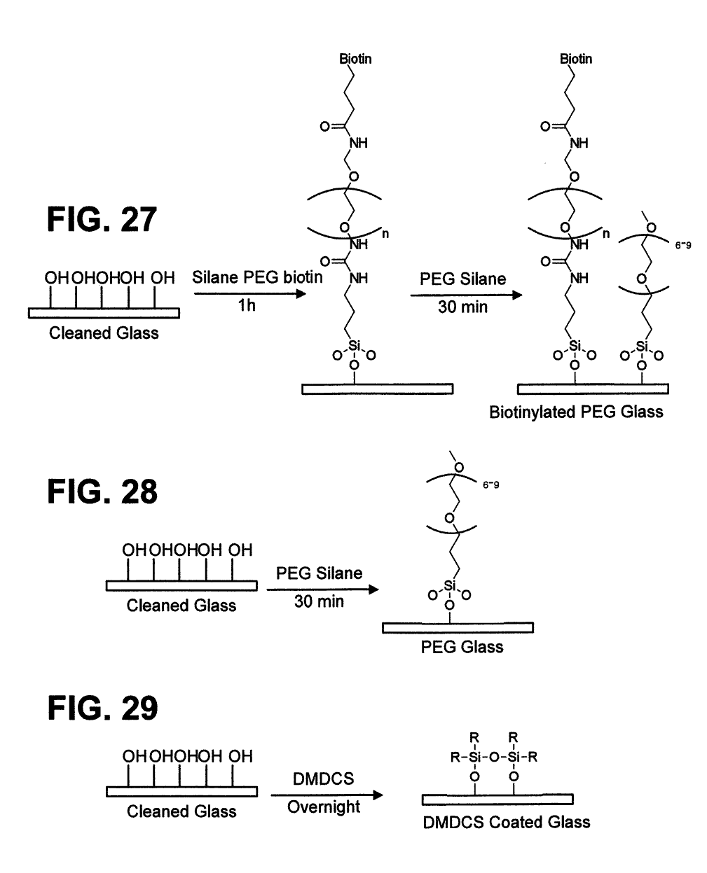

For example Biotin and PEG were grafted onto the surface of glass pedestals using a solution immersion method, as shown schematically in FIG. 27 and FIG. 28. Silane-PEG-biotin (MW=600, Nanocs) was first grafted onto clean glass pedestals by incubating in 20 mg of the reagent dissolved in 1 mL 95:5 w/w ethanol/water for 1 h at room temperature. The modified glass pedestals were rinsed three times with DI water and dried. To block any exposed/unreacted glass, neat silane-PEG (CH₃(CH₂CH₂O)ₙ(CH₂)₃Si(OCH₃)₃, n=6-9, Gelest) was then grafted onto the biotinylated glass pedestals at room temperature for 30 min, and rinsed with DI water and dried by air.

The glass pedestals may also be rendered hydrophobic. For example, a chemical vapor deposition (CVD) method was used to hydrophobize the surface as shown schematically in FIG. 29. An excess amount (1 mL) of dimethyldichlorosilane (DMDCS, Sigma Aldrich) was added into a bottle and put in a jar containing cleaned glass pedestals. The jar was sealed, evacuated under vacuum for 2 min, and heated at 60° C. for 15 min to accelerate the vaporization of the DMDCS. The jar was then stored at room temperature overnight. The glass pedestals were cleaned by sonication in toluene for 5 min to remove any unreacted silane deposited the surfaces and dried by air.

Nickel chelate resin coated glass pedestals: Nickel chelate resin is well-known to selectively bind biomolecules labeled with 6-his tags. Nickel chelated resin coated glass, 180 μm thick, was purchased from Xenopore and diced into small squares measuring 500-800 μm on a side.

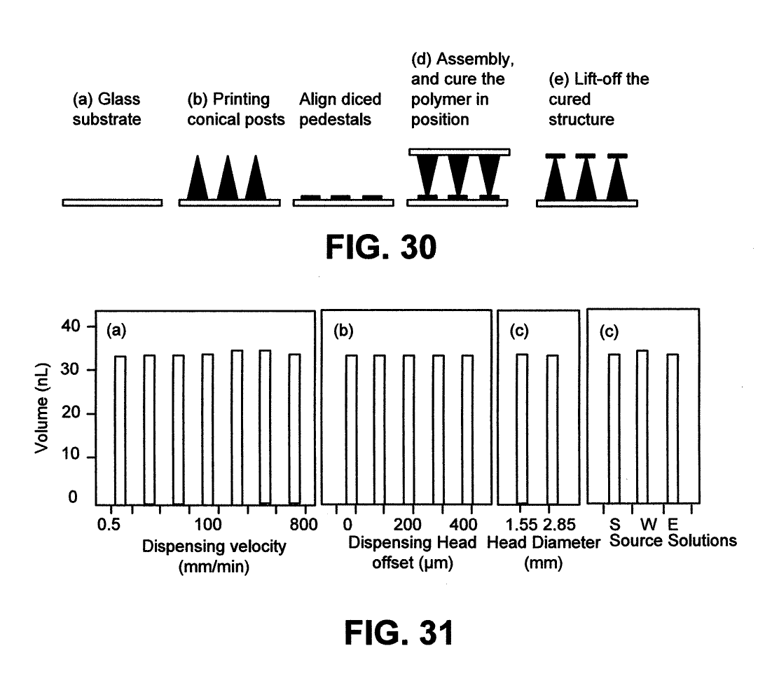

Assembly of diced pedestals onto printed conical posts: This method is based on printing and dicing technology as shown in FIG. 30. The conical posts can be made the same as before. Different from the method of FIG. 25, the caps, or pedestals are made by dicing thin substrates such as glass, silicon, metal, or other materials. The conical posts and the pedestals are assembly together. Adhesion between the posts and the pedestals can be formed during curing of the polymer posts or by adding a thin layer of adhesive. The substrate on which the posts are printed can be a glass slide or a rigid plastic or metal substrate. As discussed earlier, it could be possible to fabricate the posts by 3D printing.

The pedestals can be pre-aligned onto a plate, especially one into which depressions are formed to facilitate the alignment of the pedestals with the printed posts. Alternatively, the pedestals can be picked-up and placed on top of the posts using a pick-and-place machine such as those used to assemble small LED devices. A multi-head machine would increase throughput.

This method is especially suitable for making nDAP devices with rigid pedestals, such as glass, silicon or metal wafers. Moreover, such rigid substrates could be modified by different chemicals, such as PEG silane, silane-PEG-neutravidin and others to generate different bio-functions. By using such inorganic oxide pedestals, surface modification is facilitated and the treatment of PDMS anvils for ELISA and FLISA tests, described previously in this disclosure, would not be necessary.

Dispensing

The effect of dispensing parameters was studied using an nDAP with 600 μm diameter anvils. Dispensing experiments were conducted at a relative humidity of 85%. As shown in FIG. 31, a consistent volume of 33.3±0.5 nL (1.5% error) was achieved over wide ranges of these variables: dispensing velocity from 0.5 to 800 mm/min; offset between rod and anvil in the x, y or z direction from 0 to 400 μm; and rod diameter from 1.55 mm to 2.85 mm. These results demonstrate that the volume dispensed on a given nDAP is a function of the geometry of the surface and not the dispense parameters. Such a broad process window is crucial for achieving precise volumes without expensive positioning systems. In addition, the surface tension of the solution has only a small impact on dispensed volume as shown in FIG. 31. Dispensed volumes of solutions with very low surface tensions such as silicone oil and ethanol were almost the same as those of DI water. This demonstrates that the nDAP is compatible with a wide range of complex solutions with different surface tensions.

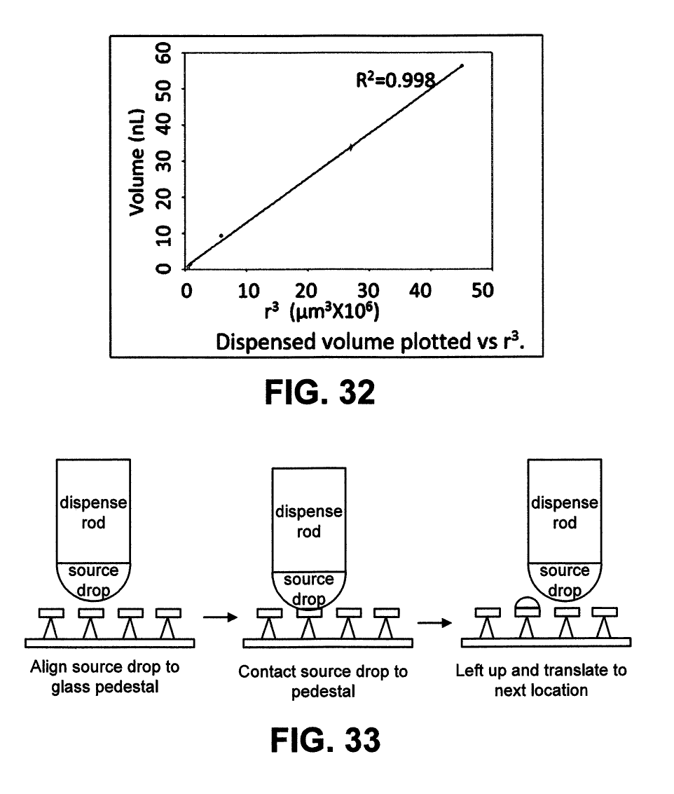

FIG. 32 depicts volume control and precision. The effect of anvil radius on the dispensed drop volume, plotted as a function of the radius cubed (r³) is shown for nDAPs with anvil diameters of 170, 200, 360, 600 and 710 μm. Droplet volume was calculated using the equation V=(Pi/6)(3 hr²+h³), where h is the height of the dispensed fluid and r is the radius. Each data point is an average of five independent measurements. The dispense volume changes linearly with r³ as r increased from 85 μm to 355 μm. This means the drop volume can be adjusted by changing the diameter of the nDAP. More importantly, the dispensing error, which is defined by the ratio between the standard deviation and the average volume, was less than 1.5% over the entire range of nDAPs measured, even down to 0.59 nL (170 μm diameter). Such a small error demonstrates a remarkably high dispensing precision.

Liquids are dispensed onto the nDAP surface by bringing a source droplet (approximately 0.5 to 25 micro-liters, more typically 5 μL), pinned to the end of a dispensing rod (diameters ranging from 0.5 mm to 25 mm o.d. with 1.55 mm o.d. being more common) into contact with the anvil surface. A low-cost robot (PRINTRBOT® Simple Metal) was modified so it could adjust the z-height and x-y position of the dispensing rod; fixtures hold the nDAP slide in place as well as a standard 96 well plate that can be used for source solution reservoirs. The robot is housed within a clear environmental chamber with an automatic humidity control system (Auberins, TH210). A computer vision guided dispensing solution was developed to automatically align, as well as set the distance between the bottom of the source drop and the top of the anvil. To transfer a droplet of precise volume from the source rod to the nDAP surface an up-down motion in the Z direction is typically used. In some cases, however, it is advantageous for the robot to translate the rod in the X-Y plane as it is lifted up off the nDAP surface in the Z direction. Complex three-dimensional curve motions were developed for mixing, washing, as well as enhancing the wetting and anchoring gel drops during dispensing. This real-time robotic dispensing system takes advantage of the dlib c++ library's structural support vector machine (SVM) training algorithm and histogram of oriented gradients (HOG) object detectors to identify and track NDAPs at 24 frames per second. The Open cv computer vision library was used to power custom algorithms for detection, tracking, calibration as well as measuring dispensed drop volume. These components are integrated into the existing software package that sets the source well position and the frequency of re-forming the source drop, thus enabling fully automated operation without manual adjustment. The system runs on a standard laptop (e.g. Acer) and uses an usb camera (Point Grey) input.

The dispensing rod attached to the robot is loaded with solution either manually, using a hand-held pipet (when sources are limited) or automatically by specifying a well of a 96 well-plate. The rod is automatically aligned to the nDAP and lowered so that the source drop makes contact with the anvil. The profile of the source drop changes as contact is made and the droplet is retracted. The dispensing process was observed with a high speed camera (Phantom, V 7.3) at 5000 frames per second. The shape of the source changes from convex to flat to slightly concave. An hour-glass shape forms at later stages of rod retraction that eventually breaks apart leaving a dispensed droplet with a specific droplet volume dispensed on the anvil.

FIG. 33 depicts a method for manual dispensing of nanoliter droplets. A drop of 10 μL source solution was loaded onto the tip of an “L” shaped polystyrene rod, using an Eppendorf pipette. The droplet was brought into contact with the glass pedestals and lifted-up by hand to transfer nanodroplets onto the nVWP surface. In the embodiment of FIG. 33, the pitch distance between adjacent posts causes a single droplet to be disposed atop each anvil. The diameter of the droplet corresponds to the top diameter of the anvil such that no droplet contacts more than one anvil.

Automated dispensing of nanoliter droplets: Automated dispensing was conducted using a robot (Janome-2203N) with a positioning accuracy off 10 μm. A polystyrene rod (3 mm diameter) was mounted on the robotic arm. The tip of the rod was polished to be flat using #600 sandpaper. A fixed volume (10 μL) of the source solution was placed onto the flat tip of the polystyrene rod using an Eppendorf pipette. The robotic dispensing process consists of three steps as illustrated in FIG. 33: the rod with source solution was aligned with glass pedestal; the rod was then moved downward at a preset speed (2 mm/s) enabling the source solution to contact the glass pedestal at fixed height such that the bottom of the droplet was brought 200 μm below the top surface of the glass pedestal; lastly, the rod was lifted-off from the surface at a pre-determined speed (15 mm/s) and translated to the next dispensing location. The dispensing processing was monitored using a microscope (CENTRITEL®, Infinity) with camera system (PIXELINK®). The images/videos were analyzed with PIXELINK® μScope software.

Manual dispensing of microliter droplets: Large droplets with volumes between 0.5-2 μL were deposited onto the 500×500 μm glass pedestals using a hand-held Eppendorf pipette. The droplet was first formed at the tip of the adjustable pipet and the bottom of the droplet was brought into contact with the top of the glass pedestal. The pipet was manually lifted up, transferring the droplet to the glass pedestal.

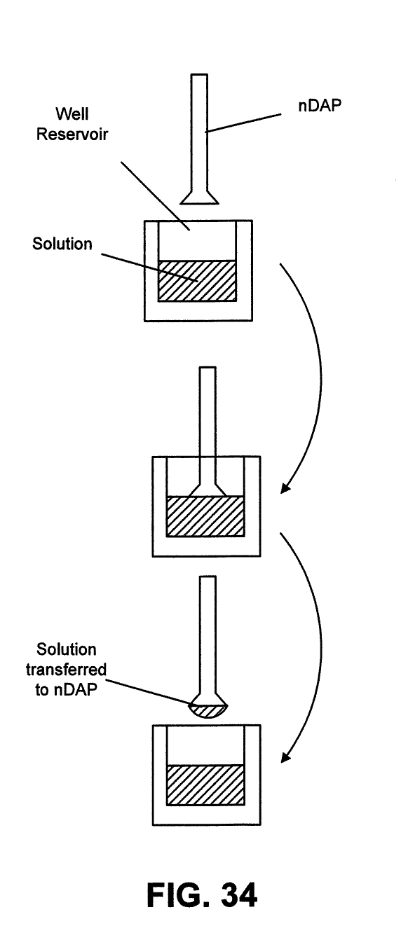

The nDAP tip can be used as a dispensing head for the precise transfer of liquids with volumes ranging from picoliters to microliters. The concept is shown in FIG. 34. In the first step, the nDAP tip is aligned with the solution reservoir. In the second step, the tip is dipped into the solution. The liquid can only wet the surface facing the liquid reservoir even when the tip is immersed deep into the reservoir. In the third step, the nDAP tip is lifted up from the reservoir and a drop forms at the end of the tip. The volume of the drop transferred onto the nDAP tip can be controlled by changing the diameter of the nDAP tip, the depth with which the tip enters into the reservoir, as well as the viscosity and surface tension of the solution. The transfer volume is constant at fixed conditions, which is very valuable for achieving high precision dispensing. The reservoir could be standard 96, 384, 1536, etc. microplates or any arbitrary container.

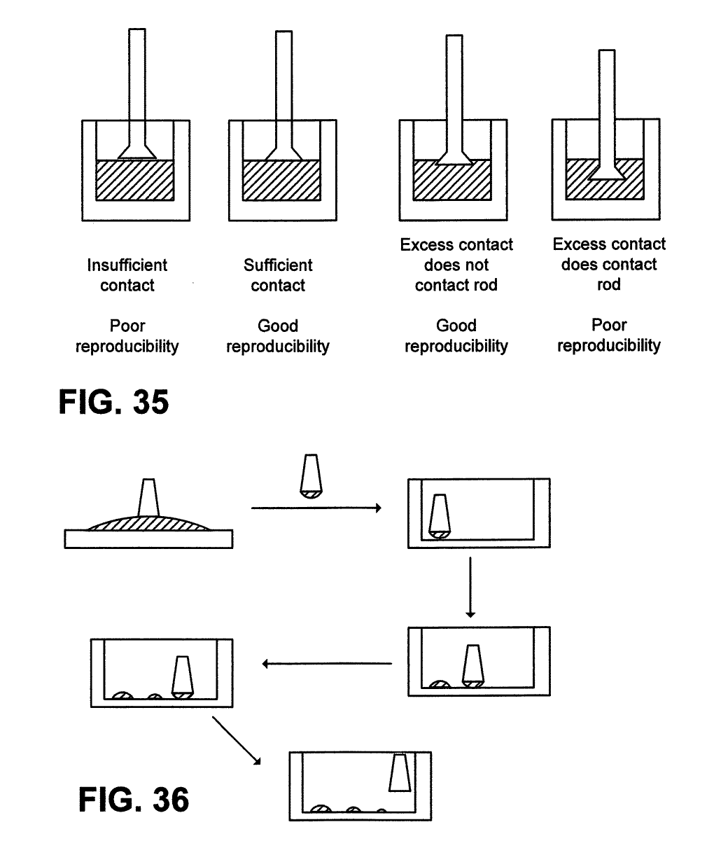

The nDAP shape is key to effective and reproducible drop volume pick-up. The wetting of the backside of the nDAP tip is prevented because of the nDAP shape. As shown in FIG. 35, liquids cannot flow around the sharp edge formed by the “anvil” shape of the nDAP tip even when the tip is fully submerged. If the tip does not contact the reservoir surface completely, an insufficient amount of fluid may be transferred as shown in the first panel of FIG. 35. Also, if the rod is immersed too deep into the reservoir, such that the fluid can flow around the anvil and contact the rod (last panel in FIG. 35), then an uncontrolled amount of fluid will attach to the nDAP dispense rod. One of the main advantages of using nDAPs for dispensing is that the dispense rod has a wide x-y-z-axis position operating window in which to pick-up a reproducible droplet volume. The rod position can go from just touching the solution, to being immersed to a depth in the range from 50 to 1000 microns below the surface of the solution in the reservoir and pick-up the same volume of solution on the tip of the rod. The depth tolerance depends upon the diameter of the rod as well as the source. For example, a 2.5 mm diameter nDAP tip can be immersed to a depth of 1 mm into a 40 μL drop poised on a superhydrophobic nDAP source substrate without wetting the back side of the tip. A smaller diameter tip can also be submerged, but to a lesser depth. For example, a 1.1 mm diameter nDAP can be immersed 700 μm below the surface of a 30 μL source drop. The large positional tolerance applies to solutions regardless of their surface tension or viscosity and offers significant advantages including: volume reproducibility, ability to use low-cost robotics, increased speed, etc.

Several methods have been developed to transfer the drop, which adheres onto the nDAP tip, onto a dry or wet substrate. For example: 1) when the substrate is dry, porous, and/or can absorb the solution, the drop can be completely sucked away by the substrate as shown in Example 3; 2) when the substrate for the liquid to be transferred is dry and relatively more hydrophilic than the nDAP tip, the drop can be fully transferred onto the substrate by continuously dispensing a few spots onto the substrate as shown in Example 4; 3) when the substrate is dry and superhydrophilic, the liquid on the nDAP can be fully transferred onto the substrate due to the strong capillary forces of the superhydrophilic surface; 4) when the substrate is wetted by a compatible solution, the drop on the nDAP can be transferred into the solution by contacting the drop with the solution surface. Due to the capillary force of the surface tension, the drop could be fully transferred into the solution when the capillary force is designed to be along the flat surface of the nDAP tip as shown in Example 5; 5) when the substrate has a bottom corner such as standard microplates and various containers, and the corner is wetted by a thin layer of compatible liquid (less than 250 μm thick), the drop can be fully transferred by contacting the edge of the nDAP tip with the corner of the well as described in Example 6.

### Example 3: Printing on a Porous Hydrophilic Substrate

Arrays (5×10) of nDAP tips made of PDMS were attached to the arm of a low-cost robot. The nDAP arrays were dipped into a glass well containing a 1% sodium alginate aqueous solution and lifted vertically to generate drops on each tip of the nDAP arrays. The weight of the nDAP arrays was measured to be 4,798.71 mg and 4,778.31 mg, respectively, before and after generating drops on each nDAP. The average volume of the drop transferred onto each nDAP tip was 408 nL, assuming the density of the solution is 1 g/cc. A nonwoven porous membrane (TexVantage, 8939) made with cellulose and polyester was attached onto a glass slide and used as a substrate for the liquid to be transferred. This membrane can absorb the alginate solution very well. The nDAP arrays with drops were brought to contact with the membrane, and all the drops were transferred onto the substrate, forming wet spots. After transfer, the weight of the nDAP array decreased to 4798.69 mg after the transfer, indicating all the liquid has been transferred onto the porous membrane.

### Example 4. Multiple Printing Method for Transferring Drops onto the Bottom of a Dry Well of a Microplate

A single PDMS nDAP tip was used to transfer a rhodamine B (RhB) solution (conc.=500 mg/L). A drop with a volume of 330 nL of this RhB solution was generated onto the nDAP using the dipping method described above. The tip was then brought to contact the bottom of a standard 96 well microplate made from polystyrene, which is more hydrophilic than the PDMS nDAP. After 3 printing contacts, all the solution was transferred onto the bottom of the wells as schematically shown in FIG. 36.

### Example 5. Transferring a Liquid Drop from an nDAP Dispense Tip to a Solution in a Well of a Microplate

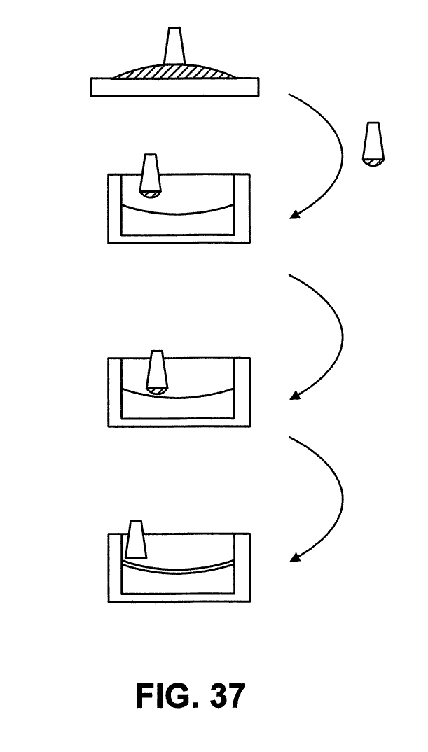

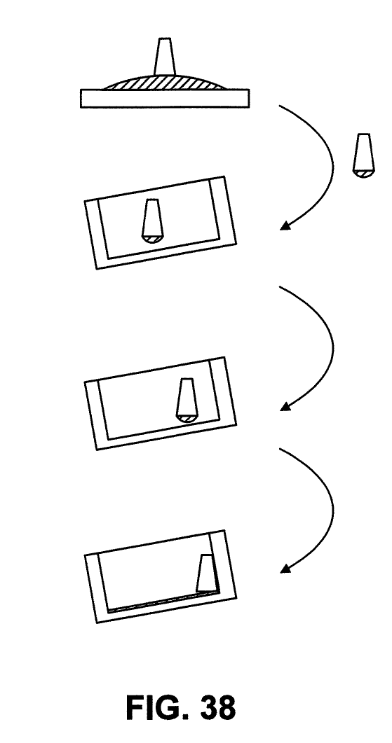

A 330 nL drop of RhB solution (500 mg/L) was formed onto an nDAP tip using a dipping method (as described in Example 4). The tip with the drop was then brought into contact with a solution contained in a standard 96 well microplate made from polystyrene. To ensure that the drop on nDAP tip is completely transferred, the edge of the nDAP tip was brought to contact the meniscus formed at the sidewall of the well as shown in FIG. 37. Due to the strong shear force generated by the solution meniscus, the flow of liquid from the nDAP tip was directed to the solution in the well along with the flat surface of nDAP tip. As a result, no liquid was removed from the well when withdrawing the nDAP tip after dispensing. This complete transfer of solution from tip to well is important for minimizing cross contamination.

### Example 6. Dispensing into the Bottom Corner of a Microplate Well

A 330 nL drop of RhB solution (500 mg/L) was formed onto an nDAP tip using a dipping method (as described in Example 4). A standard 96 well plate made with polystyrene was prepared for dispensing by washing with DI water and drying by baking in an air convection oven at 60° C. for 15 min. Because of the strong capillary force at the corners, a very thin layer of water (trace amount that cannot be seen by the naked eye) was maintained in the corner of the well, while the other areas were completely dry. The edge of the nDAP was brought into contact with the corner as shown in FIG. 38. Due to the strong capillary force, the solution was completely drawn away from the nDAP tip into the corner of the well.

Gel Types: In the examples discussed above, a sodium alginate gel solution was dispensed and subsequently cross-linked either by exposure to a CaCl₂ mist created with an ultrasonic mister (EWARE 5K119 Mini Office Bedroom Ultrasonic Humidifier) or by immersion into a CaCl₂ solution. Cross-linked alginate gel has many advantages including the ease with which it is dispensed and cross-linked, as well as the gel's compatibility with many cell types. However, some cells prefer other types of gel chemistry. Also, as discussed previously, it can be advantageous to use two different gel systems to study diffusion of molecules from one portion of the drop to another.

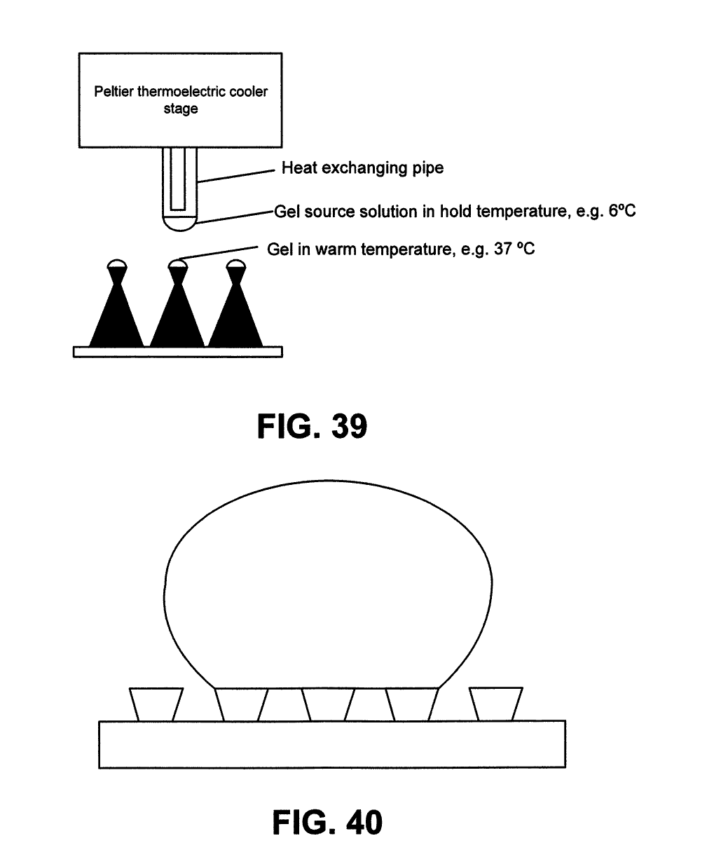

Two other types of gel that have been widely studied are collagen and agarose. These gels are temperature sensitive. At low temperatures (e.g. 5-6° C.), the molecules remain in solution. As the temperature is raised to 37° C., the molecules react forming a firm gel. Thus to dispense cells or drugs within these temperature sensitive gels, it is necessary to dispense a solution maintained at low temperature, while maintaining the nDAP substrate at a higher temperature (e.g. room temperature to 37° C.). A schematic for dispensing nano/picoliter volumes of temperature sensitive gels is shown in FIG. 39. A thermally conductive dispensing rod (e.g. solid copper or a copper heat pipe) is attached to a Peltier thermoelectric cooler to precisely control the temperature of the source drop. The nDAP temperature could be controlled with a heater positioned under the slide, or the entire environmental temperature could be controlled at the appropriate temperature (e.g. 37° C.). After dispensing, the nano/picoliter gel drops will be cured as they warm to the appropriate temperature on the anvils. Upon curing, the cells (or drugs) will be held firmly onto the nDAP posts.

After a first droplet is dispensed, a second fluid droplet can be dispensed on top of the first. There are three ways that this can be used: (a) a non-mixing all-aqueous system; (b) a non-mixing aqueous-oil system; and (c) a mixing system.

Non-mixing all-aqueous system: In this case, a gel droplet can be first dispensed and cross-linked thus anchoring the gel on the anvil. Subsequently, a second aqueous drop is dispensed on the first drop, creating what appears to be a single larger drop on the anvil. Because the first drop had been cross-linked, the droplets do not mix. However molecules in the second drop can diffuse through the gel and be trapped by reactions with cells or molecules inside the gel. This type of reaction, where a peptide diffuses through the gel and is bound to a cell is discussed in more detail in the later section entitled: “High resolution observation of reactions between peptides and cells”. Similarly, molecules that are trapped in the cross-linked gel can diffuse through the gel and enter the outer fluid layer. If traps for such molecules exist in the outer fluid layer, then the composition of these molecules can be detected and/or quantified.

In one example, cells embedded in cross-linked gel within the first droplet are known to secrete various antibodies/cytokines. A second droplet, containing beads with antigens (or other trapping agents) bound to the surface of the beads, can be dispensed on top of the first cell-containing-gel droplet. This second droplet can also contain gel and be cross-linked in order to limit evaporation. As the droplets are incubated on the surface, the antibodies/cytokines from the cell may diffuse through the gel and be trapped by the bead. By exposing the drops to the appropriate fluorescent tag, the composition and quantity of trapped species can be determined. This method can be used to identify cells that produce the desired antibodies in larger quantities than other cells of the same type. Once identified these “super-producing” cells can be cultured and propagated to make industrially relevant quantities for therapeutic applications.

In a second example, the beads in the outer drop can be removed from the nDAP surface (for example, alginate gel cross-links can be reversed by immersion in sodium citrate solution). After isolation, the beads can be quantified by flow cytometry. Alternatively, the isolated bead can be dispersed in the appropriate matrix (e.g. 2,5-dihydroxy benzoic acid or DHB) and the proteins/peptides can be removed from the bead surface (e.g. exposure to acid) and the composition of the proteins/peptides determined by MALDI-TOF. This process would have the advantage that no fluorescent tags would be required.

In a third example, the cells-in-gel are formed using an alginate gel cross-linked with calcium ions. A second droplet of a different gel, say a temperature sensitive gel, is deposited onto the first gel droplet at low temperatures and cured by warming to room temperature. After incubation, the appropriate matrix is added and allowed to evaporate. At this stage, the antibodies/cytokines expressed by the cell would be extracted by the matrix. Introduction of the nDAP into the MALDI-TOF would enable identification of the antibodies/cytokines expressed by the cell. The advantage of this technique is that no specialized treated beads are required. Moreover, all antibodies/cytokines expressed by the cell can be identified. By keeping the cell isolated from the expressed molecules, contamination from the large number of proteins and peptides that would be released during ablation of the cell during MALDI-TOF analysis would be eliminated.

In a fourth example, a relatively large amount of solution is dispensed on top of the cells-in-gel. Because of the superhydrophobic properties of nDAP surfaces, especially when the gap between adjacent anvils is less than 600 μm, large droplets (>200 nL and even greater than 200 μL) can be placed on the surface as shown schematically in FIG. 40. This large amount of solution can protect cross-linked gel nano/picoliter droplets from evaporation for hours when maintained in a humidity controlled atmosphere. The droplet can serve other purposes in addition to evaporation protection. For one example the droplet can contain growth medium which would diffuse through the gel and foster cell culturing. For another example, the droplet can contain a hybridization probe solution that could diffuse through the gel and label the mRNAs inside the cell, or proteins on the cell membrane. For another example, the droplet can contain permeabilization solution that could diffuse through the gel and permeabilize cells. For another example, the droplet can contain washing buffers, which could wash away excess bioreagents from the gel and cells. For another example, the droplet covers different cells which are isolated on adjacent posts of the nDAP substrate. This provides a special channel for studying the cell-cell interactions, and the cell-cell interactions could be turned on and off by adding/removing the solution.

Non-Mixing Aqueous-Oil System.

A droplet of an aqueous solution is first deposited onto the nDAP surface. A droplet of oil can then be deposited onto the first drop. Because the aqueous and oil phases are immiscible, there is no need to cross-link the aqueous droplet (unlike in the aqueous-aqueous case (a) described above, although dispensing an oil drop on a cross-linked aqueous gel drop may be preferred in some cases (e.g. to suppress evaporation). This method can be very advantageous for studying reactions at the water-oil interface or for diffusing an omniphobic molecule selectively from the oil phase to the aqueous phase, or from the aqueous phase into the oil phase.

In one example, a droplet of DI water was deposited onto one anvil, and then a secondary droplet of silicone oil was deposited onto the water droplet at the same location. The silicone oil could be deposited onto the water droplet and fully cover it. The drop volume on the posts was almost doubled after the secondary dispensing of silicone oil. However, the silicone oil could only slightly reduce the water evaporation rate in an 85% RH environment compared to the gel drop, alone.

Mixing System and Serial Dilutions: In some cases, it is desirable to mix two droplets together. In one case, this may be desirable in order to increase the rate of a reaction between chemicals introduced from a second droplet to the first droplet. In another case, mixing is desirable in order to more rapidly achieve uniform concentrations when a serial dilution is desired. For mixing to be enhanced, both droplets should be liquid (neither a cross-linked gel) and some form of energy introduced into the combined droplets. Examples of energy introduction include physically causing the dispensing rod to be moved (up-down and or left-right, etc.) within the combined droplet by the robot, vibrations from an ultrasonic agitation, evaporation by blowing air over the surface or thermal gradients from either heating the substrate or absorption of IR radiation.

In one example, serial dilution of a concentrated droplet of Rhodamine B (RhB) solution was achieved. Seven droplets of water (200 nL/droplet) were first placed on adjacent anvils of an nDAP substrate. An initial droplet (200 nL, 0.031 moles/liter of RhB) was placed on the dispense rod tip. This droplet was then brought into contact with the first water droplet and mixed by moving the rod up and down using the robotic dispenser. After sufficient mixing (about 3 minutes) the rod and combined droplet (400 nL total) were moved apart creating two droplets of the same, original volume (200 nL). Both droplets now had the same concentration, nominally half the starting concentration of RhB. This process was continued with the next six drops until all seven had been mixed with the drop on the rod that had mixed with the previous drop. The UV-vis spectra of these drops were recorded to determine the concentration of RIB in each drop. Due to the small volumes, each drop was diluted with 1 ml of DI water to create a sufficiently large volume to measure using reduced volume cuvettes. The intensity of the absorption peak of RhB at 550 nm for the first 3 droplets changed from 0.37 to 0.18 to 0.09 (V1, V2 and V3, respectively), which matches predictions (based on Beer's law) for 50% dilution. The other drops were too dilute and beyond the detection limit of the spectrometer.

Using Single or Arrays of nDAP Tips for Holding Source Drops

This disclosure demonstrates that the sharp, re-entrant edges of nDAP tips (also known as anvils) can exhibit good Superoleophobic properties both on single nDAP tips as well as arrays of nDAP tips. Solutions with a wide range of surface tensions can be suspended on nDAP tips, forming drops without wetting the side or back of the nDAP tips. On a single nDAP tip, a single drop can be poised. On an array of nDAP tips, a relatively larger drop of solution can be poised such that the drop is suspended on the tops of multiple nDAP tips. Such an nDAP surface can reduce the solid-liquid contact area significantly compared to normal well containers. This nDAP array can be used to hold various biochemical reagents including proteins, surfactants and non-aqueous solvents. One of the major advantages of this type of nDAP substrate is that it can be used as a source droplet substrate for subsequent dispensing. Users can place a droplet of a precious fluid that is rare (e.g. venom from spiders), expensive, or otherwise difficult to obtain, and then use an nDAP dispensing system to create multiple smaller droplets from it. After the experiment, the remaining unused solution can be easily lifted off the nDAP substrate and placed in an appropriate container (e.g. centrifuge or Falcon tube) for long-term storage. In this way, a user can pick up all of the remaining solution from the nDAP substrate, leaving no liquid behind. On conventional substrates, the drop would wet the substrate and this portion would not be retrievable (e.g. high dead volume).

The source drop substrate should satisfy two objectives. First it should hold the source drop in one position, even when the droplet is subject to vibrations (e.g. when the robot moves back and forth). Second, it should minimize the solid-liquid contact area (e.g. maintain the Cassie state) to ensure that any remaining liquid can be removed from the substrate. To fabricate an effective nDAP source drop substrate, the pitch, pattern and diameter of the nDAP tips need to be optimized as described in Example 7. For nDAP tips with a certain diameter, the larger the pitch, the lower the solid-liquid contact area. Thus at larger pitches, the mobility of a drop will increase and the ability of the drop to transition from the Cassie to the Wenzel (e.g. wetted) state will also increase. Thus the pitch can be optimized so that a drop will remain stable both in location as well as in the Cassie state. For nDAP tip arrays with a certain pitch, the larger the tip diameter, the greater the solid-liquid contact area and the more difficult for the drop to transition form Cassie to Wenzel's state.

Using arrays of nDAP tips for holding source drops: For this application, the value of D (spacing between structures) is important and can affect the ability of the surface to support a droplet in the Cassie state. An excessively low value of D may result in a drop inappropriately bridging two posts. For nDAP applications where it is desired to isolate a single droplet on a single nDAP post, the disclosed posts utilize re-entrant structures (concave) and so the ability to support an individual droplet does not rely on the compact spacing of posts or the necessity for the droplet to be supported across an array of posts.

### Example 7. Effect of nDAP Pitch and Diameter on Drop Stability

Three different nDAP arrays made of PDMS were fabricated on glass substrates. The pattern of the arrays was rectangular. The first sample has a pitch of 400 μm and a tip diameter of 330 μm. The second sample had a pitch of 400 μm and a tip diameter of 190 μm. The third sample had a pitch of 600 μm and a diameter of 120 μm. Drops (volumes=10 μL) of 1% sodium alginate aqueous solution were placed onto the three nDAP substrates. On the first substrate (400 μm pitch, 330 μm diameter) and the second substrate (400 μm pitch, 190 μm diameter), the alginate drops were maintained in the Cassie state as shown schematically in FIG. 40. The Cassie state was very stable regardless of the level of mechanical vibration applied. However on the third sample, with the greater distance between adjacent tips, the drop could easily transition from the Cassie to Wenzel state due to vibrations present in the system.

The drop on the first nDAP sample (400 μm pitch, 330 μm diameter) has the largest solid-liquid contact area and thus a relatively lower contact angle and exhibits greater Cassie state stability than the drop on the second nDAP sample (400 μm pitch, 190 μm diameter). The drops on the first and second samples were very stable and allowed the dispensing tip to pick up all the source liquid from the nDAP arrays.

### Example 8: Superoleophobic Surfaces

When the nDAP substrate is properly designed, the surface becomes Superoleophobic and can support droplets made from liquids with low surface tensions, such as oil. To make the surface Superoleophobic two factors are desirable: the nDAP shape with re-entrant surface structure and optimal spacing between nDAP posts. If the distance between nDAP posts is too large, then the oil droplet will transition from the Cassie state to the Wenzel state. If the distance between nDAP posts is too small, then a continuous, solid surface is approximated and it will be difficult to lift droplets cleanly off the surface. Surfaces were made using a commercially available PDMS resin system that exhibit Superoleophobic properties when droplets of food-grade canola oil (about 30 mN/m) were placed on the nDAP surface. Details are shown in the following table.

| | Pitch | Diameter | Array | Super- | CA | Slip | Cassie |
|---|---|---|---|---|---|---|---|
| Sample # | (μm) | (μm) | Layout | Oleophobic | (°) | Angle (°) | stability |
| 1 | 330 | 260 | Rectangular | Yes | 140 | High | Moderate |
| | | | | | | | stability |
| 2 | 330 | 190 | Hexagonal | Yes | 150 | Good | Low |
| | | | | | | | stability |
| 3 | 330 | 140 | Hexagonal | No | | | |
| 4 | 330 | 90 | Hexagonal | No | | | |

These results show that when nDAP features are printed on a 330 μm pitch, the diameter of the anvil top should be between 260 and 190 μm to achieve stable Superoleophobic properties. When the diameter is larger than 260 μm, droplet mobility decreases; when the diameter is less than 190 μm, the drop will transition to the wetted (i.e. Wenzel) state.

Long term culturing of HEK 293 cells in 3D gel drops. 3D cell culture in hydrogels simulates a natural cell growth environment and represents a more physiologically relevant environment for cells compared with 2D culture in standard petri dishes. To demonstrate that cells could be cultured in alginate on an nDAP surface, cell-gel mixtures were dispensed, the gel crosslinked, then studied cell growth as a function of incubation time. Human Embryonic Kidney 293 (HEK 293) cells were selected for this study as they are known to be compatible with alginate hydrogel. Cell culturing sodium alginate solution (from asmbio LLC) was mixed with a HEK 293 cell suspension and used as a source solution for serial dispensing. The number of cells on each anvil can be controlled by the cell density in the source solution and the volume of the deposited droplets. Droplets of 66 nL containing approximately 150 cells/droplet were dispensed onto each post (anvil diameter of 750 μm). After dispensing, the nanoliter drop arrays were cured in a 5% calcium solution for 5 min. The cross-linked nDAP was cultured in DMEM medium with 10% FBS and 1% pen/strep. The medium was exchanged every 48 h during culturing. Fluorescein diacetate (FDA) live cell stain was used to verify cell viability. The number of living (stained) HEK 293 cells on one anvil were assessed by live-cell fluorescence microscopy (10× lens) as a function of time. Living cells are clearly labeled both immediately after dispensing as well as after 3 days of culturing in medium. The two images are compilations of stacks of 26 images; total depth is 200 μm and image stacks were merged using AxioVision software. After 3 days, the distribution of cells changed such that cell aggregates were formed and grew. In addition, HEK 293 cells can be cultured and grown for over 10 days in gel on nDAPs.

This experiment also demonstrates that a strong anchoring force exists between the nanoliter gel drop and the anvil. The forces generated during medium exchange and imaging did not cause the gel droplets to be displaced or damaged.

Single cell detection in 3D gel drop via parallel dispensing. When multiple cells are contained in one relatively large droplet, it can be difficult to track each individual cell. To facilitate single cell studies, a low concentration of cells were isolated in smaller droplets. A parallel dispensing approach was used to prevent excess evaporation. A dispersion of HEK 293 cells was used at a lower density (5×10⁵ cells/mL) as the source solution and an nDAP substrate with smaller anvils (250 μm diameter, 496 post array, 1 mm pitch). The volume of the gel drop on each post was measured to be 2 nL. An average of 2 HEK cells per drop was achieved. Using serial dispensing on 700 μm anvils, an average of one cell/anvil was achieved with the anticipated Poisson distribution. To detect the cells, FDA live cell staining (3 μM) was monitored in-situ as a function of time. Fluorescence images were recorded every 5 seconds. Cell fluorescence becomes visible after 20 seconds and reaches full saturations after 10 minutes. This study demonstrates that single cell level detection could be achieved by dispersing cells in gel that are subsequently anchored onto an nDAP and cross-linked. Smaller diameter anvils, such as 100 μm or 50 μm would further improve the spatiotemporal resolution. This FDA live cell staining study also demonstrated that small molecules (<500 Da) can diffuse quickly through alginate, react with suspended cells and be detected.

Diffusion of proteins through gel—Effect of gel density. The ability of high molecular weight proteins to diffuse through the 3D gel environment is important for studying the effect of biological drugs on cells as well as the identification of antibodies or cytokines expressed by cells. Both require rapid diffusion of high molecular weight molecules through the gels. Nano/picoliter gel drops require shorter diffusion times than bulk gels because of the shorter distances; however the cross-link density of the gel affects pore size and thus diffusion rates. To demonstrate protein diffusion as a function of gel crosslink density, a bovine serum albumin (BSA, MW=66.5 KDa) labeled with Alexa 488 was used. Sodium alginate solutions with 0.5%, 1% and 2% w/v were used along with CaCl₂ solutions of 1% and 5%; cure time was fixed at 5 min. BSA could not diffuse more than about 20 microns through the concentrated 2% w/v alginate gel cured with 5% CaCl₂ after 24 hours. In contrast, a 0.5% w/v sodium alginate solution cured by 1% CaCl₂ was highly permeable. Gel droplets formed from 1% w/v sodium alginate solution and cured with 1% CaCl₂ shows the best overall properties. BSA (40 μg/ml) can diffuse uniformly across the gel drop (600 μm diameter) within hours. These experiments show some BSA was able to diffuse through the gel after 0.5 hours, with the intensity of BSA progressively increasing after 1 and 7 hours. The bright periphery indicates, not surprisingly, that BSA accumulates on the external gel surface.

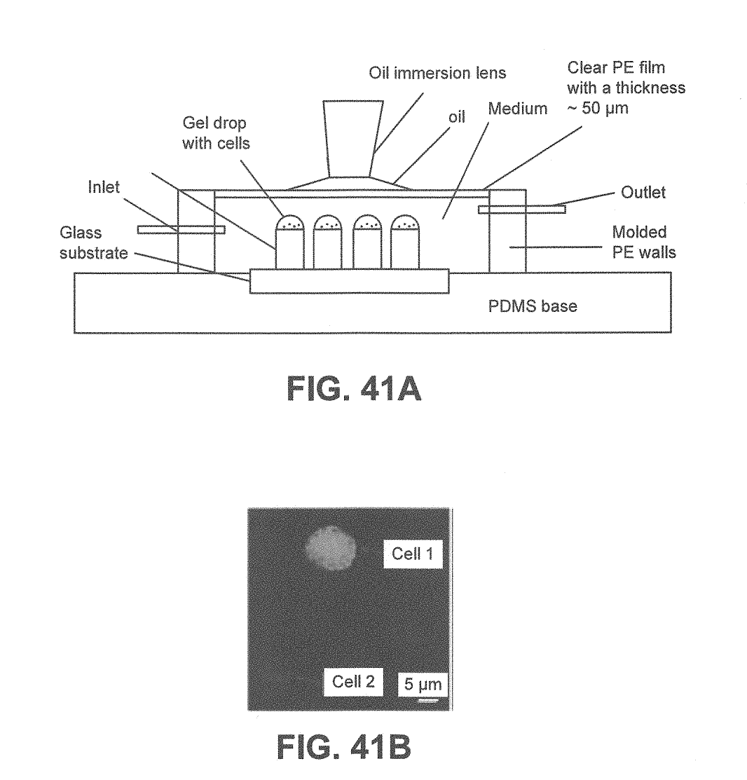

High resolution observation of reactions between peptides and cells. Not only should proteins and peptides diffuse through the gel, they should also be able to react with cells, for drug screening applications. A cell permeable peptide (Arg)₉ was used, FAM-labeled (MW=1782 Da) to demonstrate diffusion through gel and penetration into HEK 293 cells. Sodium alginate solution (1% w/v) was mixed with a HEK 293 cell suspension and used as a source solution for single dispensing on 750 μm diameter nDAP anvils using a single robotically controlled rod. The alginate was cured with 1% calcium chloride for 5 min. As shown in FIG. 41A, the nDAP was enclosed in a specially designed holder incorporating a thin polyethylene (PE) window that allows high resolution imaging and solution exchange. The holder was filled with medium and imaged using a confocal microscope (CLSM). After the cells were imaged, a 15 μg/mL solution of (Arg)₉ was introduced using a syringe, displacing the medium. After 5 mins of incubation, CLSM images were recorded using a 63× oil immersion lens as shown in FIG. 41B. Two cells with cell permeable protein were clearly seen. The holder enables observation of single cells at high resolution while permitting the exchange of solutions without interfering with the gel anchor on the nDAPs.

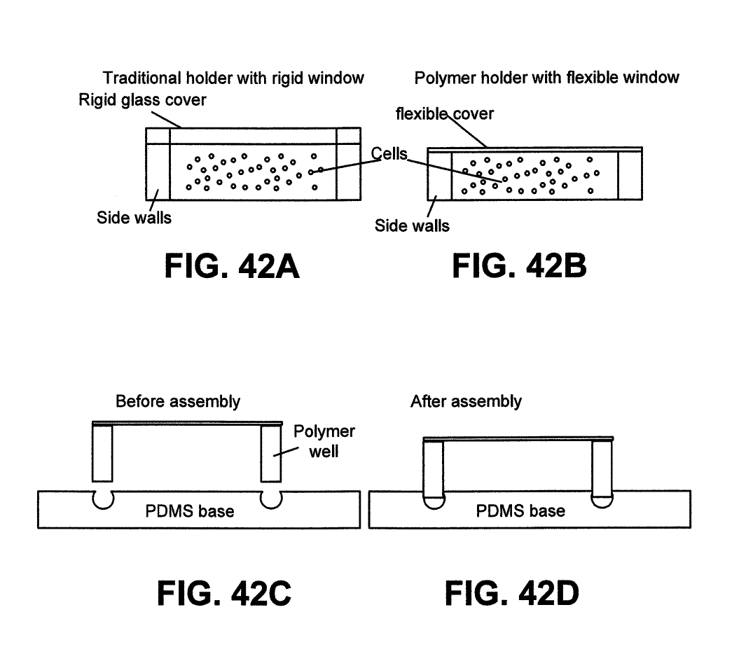

Holder for high resolution microscopy. A traditional holder for biomedical imaging is composed of a glass coverslip and a soft (e.g. PDMS) frame as shown in FIG. 42A. The glass coverslip typically has a thickness between 100-150 μm, depending on the lens with which the coverslip is to be used. Such thin layers of glass are very rigid and fragile. They are designed for being placed in direct contact with the cells of interest that are grown directly on a microscope slide or petri dish.

However, when coverslips are used to image cells embedded in gel on an nDAP surface, the rigidity and thickness of the coverslip can limit the depth into the gel that the lens can focus. Thus the lens cannot be moved close enough to the cell to image it at high magnification. For example, the 63× oil immersion lens has a focus length of ˜190 μm, and the glass coverslip can limit the imaging depth to be only 40-90 μm. Only a depth of 4-9 cells could be imaged in the best case assuming a cell diameter of 10 μm. To solve this blocking problem caused by the traditional rigid coverslip, a polymer holder with a thin, flexible window was developed. Schematics of the polymer holder are shown in FIG. 42A and FIG. 42B. The polymer holder is composed of a thin and flexible cover and a frame with four side walls. The polymer cover has a high transmission in UV-Visible wavelength range. It was demonstrated that an image depth of 2 mm for the 63× oil immersion lens can be achieved using a polyethylene holder with a 100 μm thickness cover, which is over >20 times deeper compared to the rigid glass.

The thin flexible cover could either be formed monolithically with the frame or bonded onto a pre-fabricated frame. In either case, a good seal between the flexible cover and the frame is essential so that the solution surrounding the nDAP (e.g. water, PBS buffer, growth medium) is retained and does not leak.

The thickness of the cover could be 200 μm, 100 μm, 50 μm, or even thinner depending on the materials and the applications. Polymers having flexibility and a good optical transparency (e.g. >90%) in the UV-visible, such as PET, PP, PE, PMMA, PS, FEP, etc, could be used to make the cover.

The frame could be made from the same material (monolithic with the cover) or from a different polymer. The walls should be sufficiently wide so as to ensure good adhesion between frame and cover. In addition, fluid inlets/outlets could be incorporated into the walls to enable the exchange of the solution. In one case, the medium would be exchanged through these inlets/outlets to enhance the long-term culturing of cells. In another case, a probe compound, such as a fluorescently labeled cell penetrating protein, could be introduced while the nDAP with frame and cover are being observed in the microscope. In this way, the diffusion of the probe through the gel and its interaction with the cell can be studied in real time. The high resolution capabilities of this holder provide a significant advantage. For example, it allows the user to observe the location on the cell where the probe binds (e.g. cell membrane vs cell nucleus). The fluid inlets can be molded into the walls, or sharp needles can be pushed through the walls forming a seal and connected to a syringe or syringe pump for the introduction of fluids. The pitch of the nDAP anvils can be adjusted to influence the flow rate and flow distribution when solutions are added into the holder. For example, by imposing variable width channels between rows of anvils, variable flow rates can be obtained and thus the amount of drug to which an individual cell is exposed will be varied depending upon the cell's location within the array.

Sealing the holder to the base. The frame and flexible cover can be made separately and bonded to the nDAP base after gel droplets are formed and cross-linked. Alternatively the frame can be integral with the nDAP base and the cover applied after gel droplet formation. One approach to creating a seal between the frame and the base is by using a PDMS base with a groove and gasket molded in place as shown in FIG. 42C and FIG. 42D. The molded holder, with rigid sidewalls, can be physically pressed into the molded PDMS base such that the holder walls are inserted into the grooves. A liquid-tight seal is formed between base and holder due to the shape of the groove and compliance of the PDMS rubber. The glass slide is contained within the PDMS.

Drug Testing—Cells in Gel vs Drugs in Gel: The nDAP technology is an effective platform for observing the interaction between cells (either individual cells or cell agglomerates or spheroids) and drugs. In the examples discussed so far, cells have been immobilized in the nDAP gels. Drugs could be applied by: dispensing a drug solution droplet over the cells-in-gel using the overlay approach; introducing a drug solution in the holder; or immersing the entire nDAP into a solution of the drug. The method selected could depend upon the availability of the drug and/or the ease of conducting the experiment. In either case, the drug would diffuse from the solution into the gel and finally interact with the cell. During incubation, the viability of cells exposed to different drugs, or different concentrations of the same drug, could be monitored as a function of time, other environmental conditions (e.g. temperature, pH, etc.) as well as concentration (cell concentration and/or drug concentration).

Alternatively, the drug can be immobilized in the cross-linked (i.e. solidified) gel droplets and the cells could be added adjacent to the drug-in-gel droplets. The cells could be introduced in the same way as described above for the drug; methods of introducing cells adjacent to gels containing drugs include: overlay, flow-through holder and total immersion.

Drug delivery device: The solidified gel droplets could be used as a drug delivery device. When either drugs or cells that produce antibodies (or other useful compounds) are immobilized in gel, the therapeutic compound will be able to diffuse through the gel and enter the surrounding environment or tissue. To treat some diseases, it may be preferable that very small gel droplets are injected into the area of interest. In other cases, it may be preferable to implant an array of PDMS supported gel droplets into the appropriate location. By placing the device in close proximity to a tumor, the drug would be targeted to the correct location. Cells in gel may offer the opportunity to incubate the cells (e.g. T or B cells with the required genetic type) within the body and have the newly formed cells grow and slowly release from the gel so that a constant source of powerful cells could be available to combat the disease. The nDAP could be used either as a means to fabricate small gel droplets for drug delivery (such that after the gel droplets are formed and cross-linked, they are removed from the nDAP and collected) or the nDAP surface containing the immobilized therapeutic agent(s) could be embedded into the body as part of the drug delivery device. In the latter case, the nDAP posts and anvils would be formed on a compliant biocompatible substrate such as PDMS or TEFLON®. Many gels are biocompatible as well as biodegradable and approved for human use.

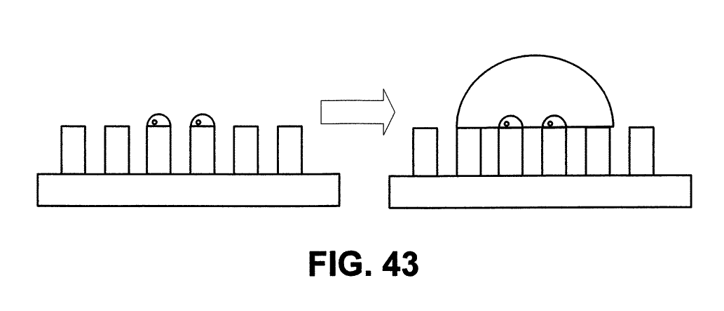

Cell-to-Cell Communication: In some cases, it is of interest to determine the molecules that one cell releases to trigger the activity of a second cell. However, it can be challenging to isolate these compounds. The nDAP platform can be used to isolate and determine the composition of these compounds. In one example, cells are immobilized in gel deposited on adjacent anvils as shown in FIG. 43. A volume of an appropriate fluid (e.g. buffer, cell medium, blood plasma, etc.) is placed on top of these cell-in-gel droplets enabling communication between the two. This fluid can then be collected and analyzed (e.g. by MALDI-TOF) to determine the composition of these compounds. The superhydrophobic properties of the nDAP surface enable the added fluid to bridge between anvils, without wetting the full surface. This aspect helps reduce the amount of added fluid required and increases the concentration of the molecules (e.g. peptides and proteins) in the added fluid, thereby facilitating detection of small quantities produced by the cells.

The nDAP surface used for such studies should be appropriately designed such that the distance between adjacent posts is small enough such that the surface tension of the fluid can bridge across the distance. For example, an anvil pitch of 1 mm with an anvil diameter of greater than or equal to 400 microns (resulting in a gap of less than or equal to 600 microns) is sufficient to maintain aqueous solutions in the Cassie-Baxter (i.e. superhydrophobic) state. Smaller gaps will support lower surface tension fluids or provide greater stability of the Cassie-Baxter state for aqueous fluids.

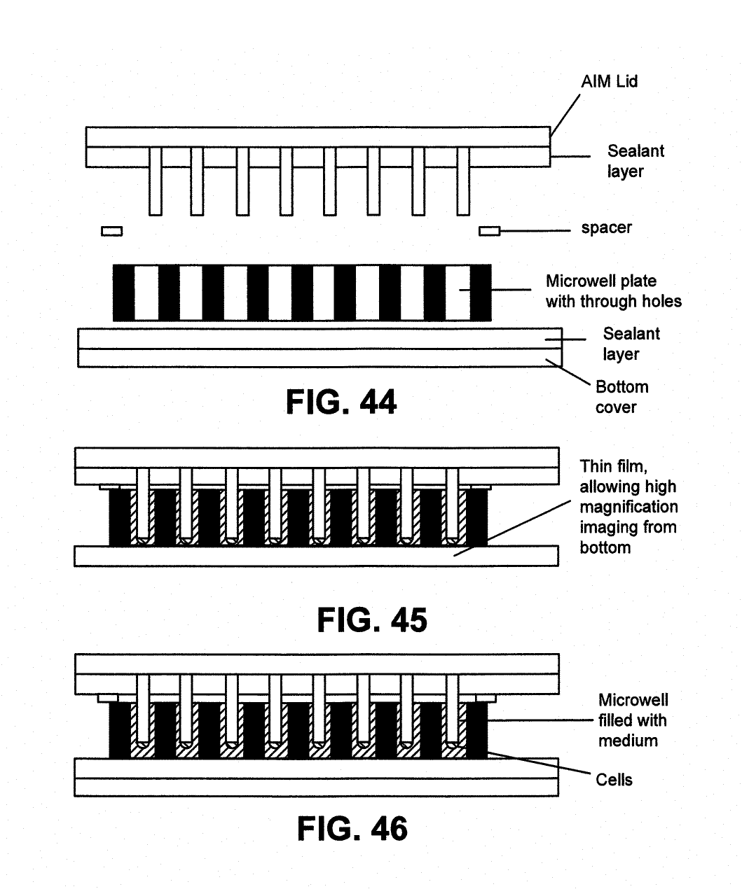

FIG. 44 depicts one embodiment where an nDAP array is formed on the lid. When higher magnification (such as 63× or 100×) imaging is required, the bottom cover could be a thin polymer film or glass sheet that is glued onto the bottom of the microwell with through holes (see the embodiment of FIG. 45). The thinner the film/glass is, the larger the imaging depth (i.e. depth of field) would be. Polymer films could be as thin as 3 μm, 12 μm or 25 μm and have much better elasticity than glass coverslips which typically have a thickness around 100 μm. In one embodiment, the system is inverted and the thin film is subsequently perforated or peeled away.

Once the nDAP lid is formed, droplets can be dispensed onto the lid. This can be accomplished by a variety of techniques. For example, an nDAP rod can be incorporated into a robot which is programmed to dispense material from a source (e.g. a drop on an nDAP substrate or a well of a microplate) to each nDAP tip on the lid in a serial manner. Alternatively, the drops can be dispensed in a parallel manner by dipping the lid into a single reservoir (with dimensions as large as the lid) such that every nDAP tip has the same solution dispensed. By circulating the solution through this reservoir, settling of denser materials (e.g. cells, semiconductor die, crystals, etc.) can be prevented and the density of particles in each drop will remain consistent. Alternatively, the lid could be dipped into a microplate with the same number of wells as tips on the lid. In this way, each nDAP tip could have a different composition that corresponds to the composition of the well of source microplate. If each well of the microplate was filled with the same solution, each nDAP location would also be identical.

For example, the system disclosed in FIG. 46 can be used as a microwell plate system to grow tissues. The cell or tissue droplets anchored on the AIM lid are immersed into the medium. The small gap maintained between the AIM lid and the microwell plate by the spacer allows gas exchange during culturing. In some embodiments, the spacer is omitted for short term applications. The thickness of the spacer can be changed according to the volume of the medium and the distance from the bottom. Medium can be changed directly by lifting the AIM lid with microtissues/cell droplets attached and placing it onto a new microplate filled with fresh medium. This simple process eliminates the risk of damaging or losing fragile tissues and greatly simplifies the medium change procedures used in commercially available systems where a pipette may be used to remove old medium without disrupting or dislodging the cells under culture. The simplicity by which cell cultures can be transferred to fresh medium is especially valuable for automating 3D cell cultures.

In the set-up shown in FIG. 45, the bottom cover with a sealant layer is used to seal the medium. The sealant layer could be varied between a soft gel (penetration of 10 mm or more) to a rubber material with the hardness in a range of a Shore 15 OO to Shore 70 A. Clamping forces can be applied to ensure a leak-free seal if a rubber material with higher hardness is used. With this set-up (FIG. 45), the hydrogel-cell drops could be imaged from the bottom under objectives with relatively low magnification (such as 5×, 10×, 20×, 40×) and a working distance larger than 1 mm.

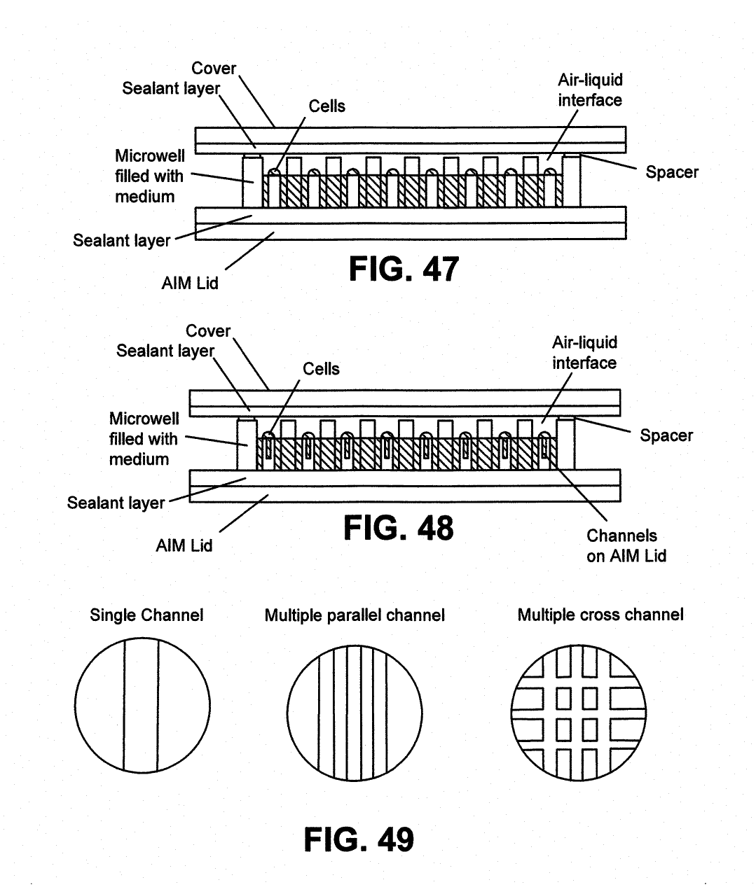

For culturing special types of cells, such as skin cells, the system disclosed herein can be flipped over as shown in FIG. 47. The sealant layer on the AIM lid prevents the medium from leaking out of the microwells, and the spacer could be moved (or a new spacer used) between the top cover to allow gas exchange. The wells can be partially-filled so that the liquid-air interface can be self-generated at the correct level by flipping over the culturing system. The volume of medium added to the microwell may be adjusted to the height of the posts attached to the AIM lid so that the liquid level achieves the correct height. This permits the lower portion of the cells-in-hydrogel to be immersed in medium while the top surface of the gel drops is exposed to air.

Microchannels can be formed into the tip of the AIM lid as shown in FIG. 48. The channels can assure the continuous delivery of medium to the microtissues. This could be especially useful when the liquid-air interface is formed in the microwell system.

The microchannel on the AIM lid can be made either by molding at the same time when manufacturing the AIM lid, or cutting with a programmable saw after the AIM lid is made. Various patterns, including multiple parallel and/or cross channels as shown in FIG. 49, could be made on the AIM lid. The width, depth and length of the channel can be varied depending upon the diameter of the AIM Lid posts, the tissue size, etc.

Although a clamp may be added to further prevent leaks, or to facilitate transport, no clamps were used, or required, in the following leak-test examples.

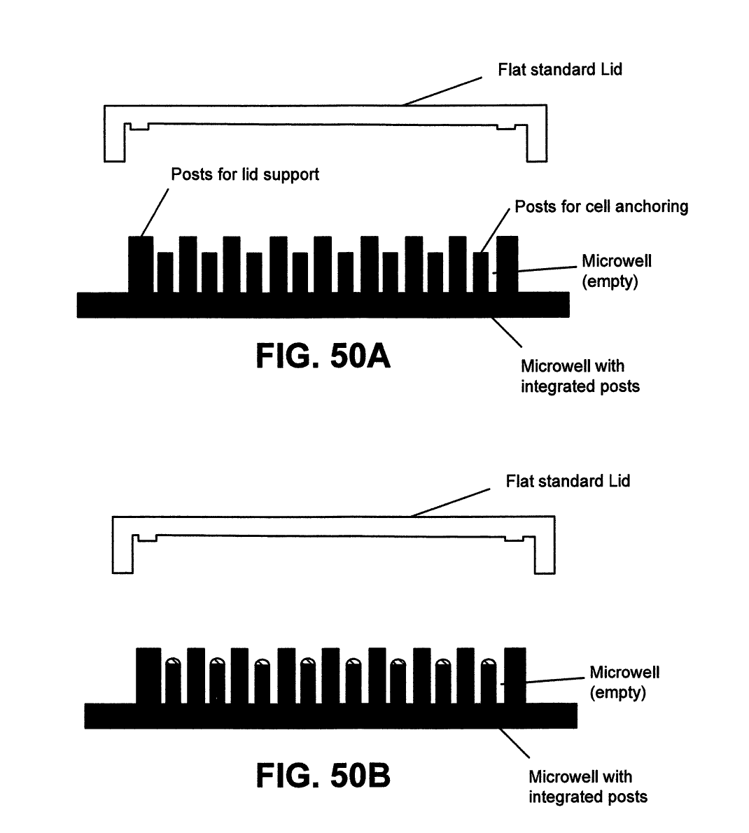

In one embodiment, the lower plate and microwell are monolithic. One such embodiment is depicted in FIG. 50A. A monolithic plate with integrated microwells and posts is provided. The plate has short posts for cell anchoring with each short post within a cylindral well. Long posts can be used for lid support.

In FIG. 50B, cells are added to the planar top surfaces of the short posts. In one embodiment, the cells are added by pipette. The cylindrical walls of the well, or the long posts, are sufficiently long to prevent the lid for contacting the cells when the lid is closed.

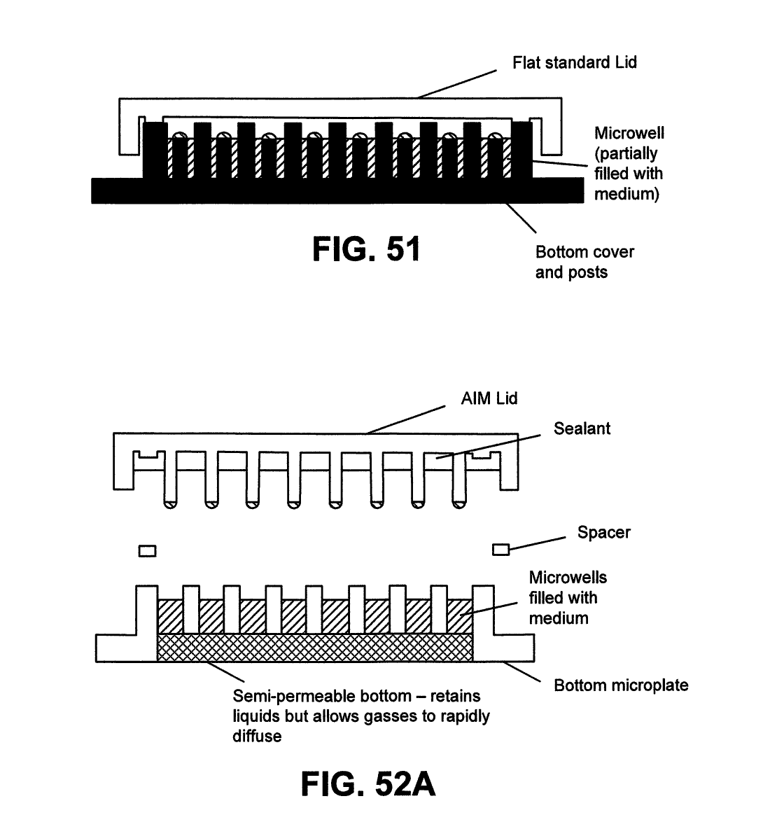

In FIG. 51, the microwells are partially filled with a medium as described elsewhere in this specification. Thereafter, the lid is closed by resting it on the cylindrical walls of the wells, or long posts. In the embodiment of FIG. 51, the medium contacts the bottom of the cells but leaves the top of the cells exposed.

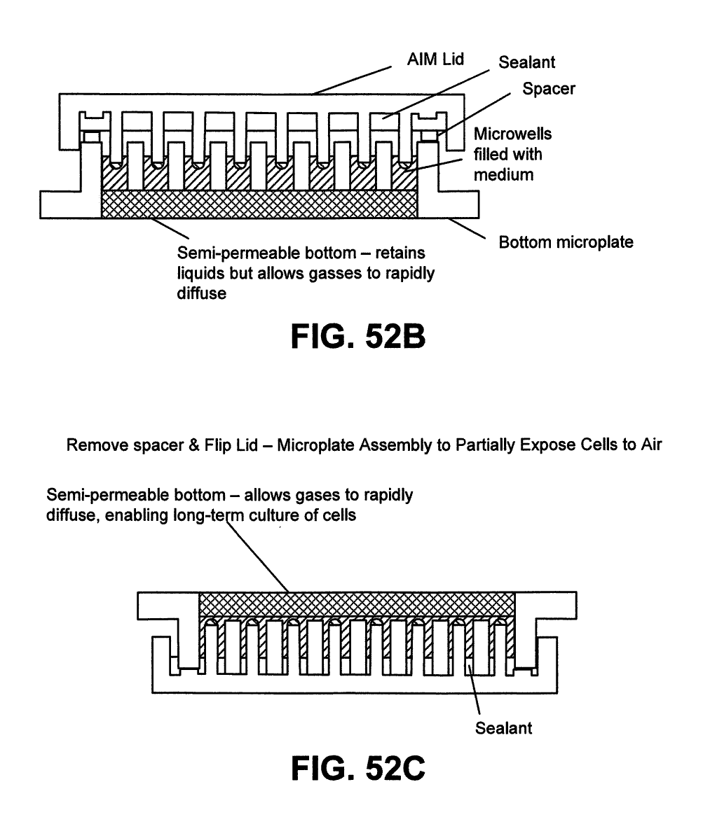

In another embodiment, shown in FIG. 52A, the lower plate and microwells are monolithic but the lower plate comprises dissimilar materials such that a porous membrane is provided on the bottom surface. Although the porous membrane distorts the optical path (thereby hindering the use of this embodiment for imaging) the porous membrane permits gases to diffuse into the medium. In one such embodiment, the pores in the porous membrane are less than 1 micron such that the liquid medium does not leak out. In FIG. 52B the cells are immersed into cells. In FIG. 52C the spacer is removed and the device is inverted such that the cells are partially exposed to air and partially exposed to the medium.

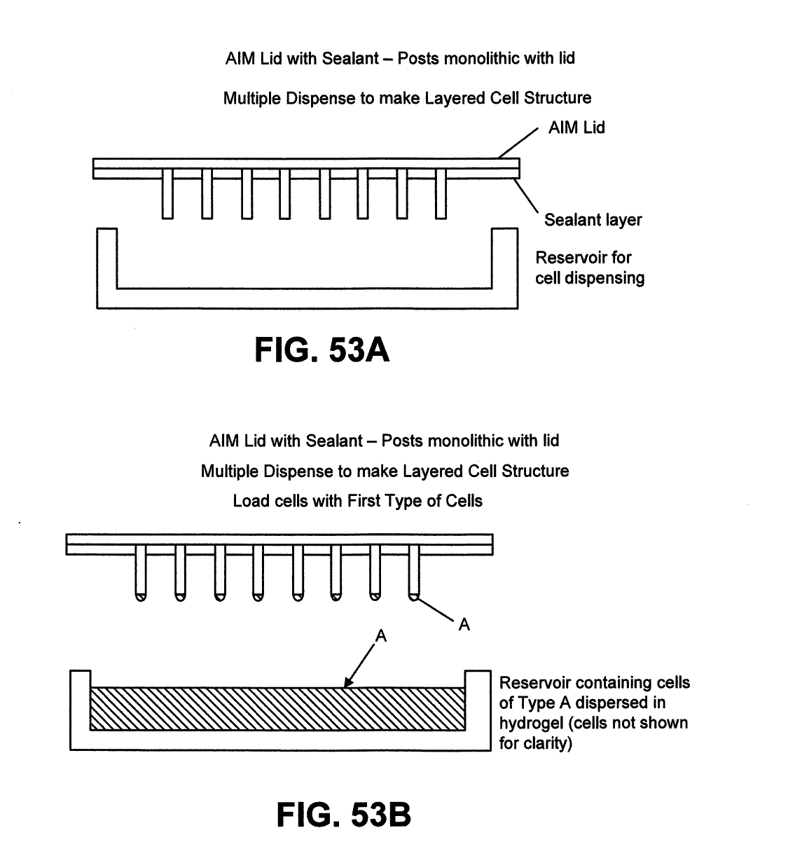

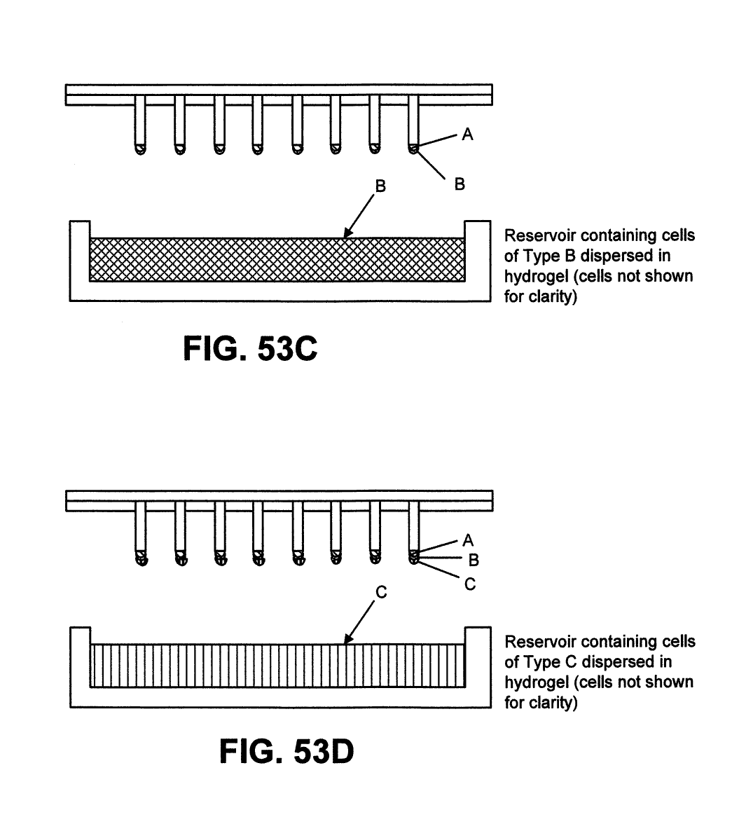

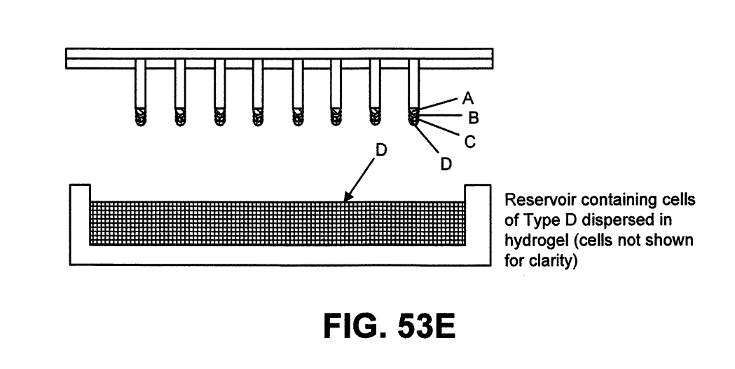

In one embodiment, the system is configured to provide multi-layer cell culturing. See FIG. 53A. This can be important for culturing of skin micro-tissues, as well as other types of cell diagnostics and micro-tissue culture. Skin tissue is comprised of layers of different types of cells. These skin cell types can form on their own by differentiation from primary cells. However, the process can be accelerated by dispensing layers with different types of cells in each layer. This gives the micro-tissue a head start. Any arbitrary number of cell types can be stacked, one on top of another. The hydrogel solution, in which the cells are dispersed adheres to the previously deposited and cross-linked layer. Alginate hydrogels do not exhibit strong inter-layer adhesion but collagen hydrogels adhere well to each other. A method for multi-layer cell culturing is illustrated in FIGS. 53B to 53E. The posts are sequentially dipped into media comprising different cells (e.g. A, B, C, D) which provides different layers of cells on the posts.

### Example 9

15 g silicone rubber was prepared by mixing the silicone resin (SYLGARD® 184 silicone, from Dow Corning, viscosity of 3500 cps and hardness of 43 Shore A) with curing agent in a 10:1 ratio. The mixture was degassed and poured into an AIM lid; the walls of the lid constrained the resin. The resin was degassed under vacuum for 12 hours, and then baked at 50° C. for 5 hours to fully cure the silicone. The cured silicone adhered well to the AIM lid and used as the sealant layer.

Dulbecco's Modified Eagle Medium (DMEM) with 10% Fetal Bovine Serum (FBS) was used to test the quality of the seal formed by the AIM lid with the sealant. The medium was added into the wells of a standard 96 well microplate. A few of the wells were half-filled with medium. Then the AIM lid with the sealant was put onto the microplate such that each tip of the AIM lid was inserted into each well. The set-up was flipped up and down for ten times, and was put on a flat bench top with the AIM lid at the bottom and stored at room temperature. The liquid-air interface was formed in the half-filled wells. No leaking was observed over 5 days.

### Example 10

The bottom of a standard 96 well-plate was removed using a saw. A thin polyester film with a thickness of 25 μm was glued onto the bottom of the well using RTV silicone 3140 from Dow corning, and cured overnight. After the curing, the Polyester film adhered to the bottom of the well very well. The medium (same as the one used in example 9) was loaded into the wells and stored for a few hours. No leaking was observed during storing.

The AIM Lid with the sealant made in Example 9 was placed onto the top of the microplate made with the thin polyester film adhered to the bottom. High magnification imaging using a 63× oil immersion lens was demonstrated. The imaging depth was 125 μm larger compared to the glass coverslip with a thickness of 150 μm.

### Example 11

10 g silicone was prepared by mixing the silicone part A and part B (LIQUAGLASS® from Castaldo with a nominal viscosity of 1200 cps and hardness of 50 Shore A) at 1:1 ratio, and the mixture was poured into an AIM lid, degassed under vacuum for 1 hour, and then baked at 50° C. for 3 hours to fully cure the silicone. The cured silicone adhered to the AIM lid very well and was used as the sealant layer. Dulbecco's Modified Eagle Medium with 10% Fetal FBS was used to test the sealing effect of the AIM lid with the sealant. The medium was added into the wells of a standard 96 well microplate. The AIM lid with the sealant was placed onto the microplate with each tip of the AIM lid inserted into a single well. The set-up was flipped up and down ten times and was then placed on a flat bench top with the AIM lid at the bottom and stored at room temperature. No leaks were observed over 1 day.

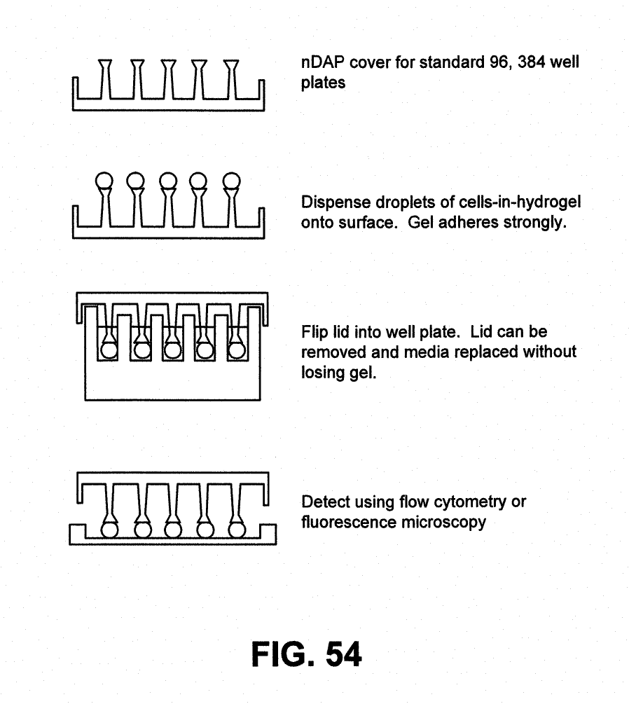

Once the droplets are dispensed on the nDAP lid, the droplets can be inserted into a microplate that contains the appropriate solution. In this way, precise volumes can be accurately and easily dispensed without cross-contamination. If the droplets contain gel, the gel is first cured (e.g. immersed in CaCl₂ solution, warmed to 37° C. or some other method depending on the type of gel used). Immobilizing cells into a 3D gel environment can be especially advantageous for some drug screening tests. After cure, the cells-in-gel anchored to the nDAP lid are then immersed into the appropriate solution in a microplate. This approach is illustrated in FIG. 54. FIG. 54 is a schematic of the use of an nDAP lid for cells immobilized in gel droplets cultured in standard microplates used for drug screening applications. After use, the cells can be imaged while remaining within the wells of a microplate, or released from the gel and analyzed by flow cytometry. Alternatively, the lid can be removed and imaged separately.

After the appropriate incubation time, the wells can be analyzed using standard techniques with the lid either in place or removed depending upon the test. For example, the lid can be placed into wells that dissolve the gel. After the cells are liberated from the gel, they can be analyzed by standard techniques such as flow cytometry, fluorescence, chemiluminescence, UV-vis spectroscopy, etc. Alternatively, the cells in gel can be imaged external to the microplate. For example, they can be imaged using confocal fluorescence microscopy using either face-up or face down presentation. If necessary, the cells can be placed into a special holder such that the gel surface is separated from a high magnification oil immersion lens by a thin layer made from a glass coverslip or a thin polymer membrane. Because the gel is flexible, the thin membrane polymer membrane may be especially effective as it would enable the microscope to focus at different depths within the gel.

Another approach to populating droplets on the nDAP lid is to insert the lid into an acoustic drop dispenser, such as the Echo Liquid Handler manufactured by LABCYTE INC. of Sunnyvale, Calif. Droplets of any volume (e.g. increments of 2.5 nL) can be transferred from the source well plate onto the nDAP lid. The advantage of using the Echo system is that any combination of volumes and sources can be used without any opportunity for cross-contamination. This flexibility would greatly enhance usefulness of the nDAP lid for certain applications (e.g. varying the concentration of cells or molecules in a drop or varying the ratios of several different chemicals/cells within a series of droplets. The ECHO could deposit the liquids directly onto the nDAP tip surface. Alternatively, gel could be first dispensed onto the nDAP lids (e.g. using serial or parallel dispensing) and the nDAP lid populated with gel drops could be put into the ECHO system for dispensing additional molecules. The gel on the nDAP lid can be cured either before or after inserting the lid into the ECHO for additional dispensing depending up the desired properties. For example, by inserting uncured gel into the ECHO, the dispensed nanoliter droplets can be mixed with the components of the gel (via diffusion) and subsequently cured. In this way, diffusion of these molecules out of the gel (after immersion into a microplate well) could be slowed, depending upon the size of the molecules and the pore size of the gel after curing.

Another advantage of using the ECHO system to dispense onto an nDAP lid is that the ECHO system can dispense only into dry, empty wells, or small wells that contain only a limited volume of solution. Since the capture well may be facing downwards, a large volume of solution will leak out during dispensing. By using an nDAP lid, the captured droplet (dispensed from the ECHO) will adhere onto the anvil-shaped nDAP tip and so will not leak away. After dispensing, the lid can be immediately placed into a filled microplate. In this way, microplates can be prepared that contain all the required solutions, and the final small volume of reagents can be added at the end, using the nDAP lid, without inverting the microplate.

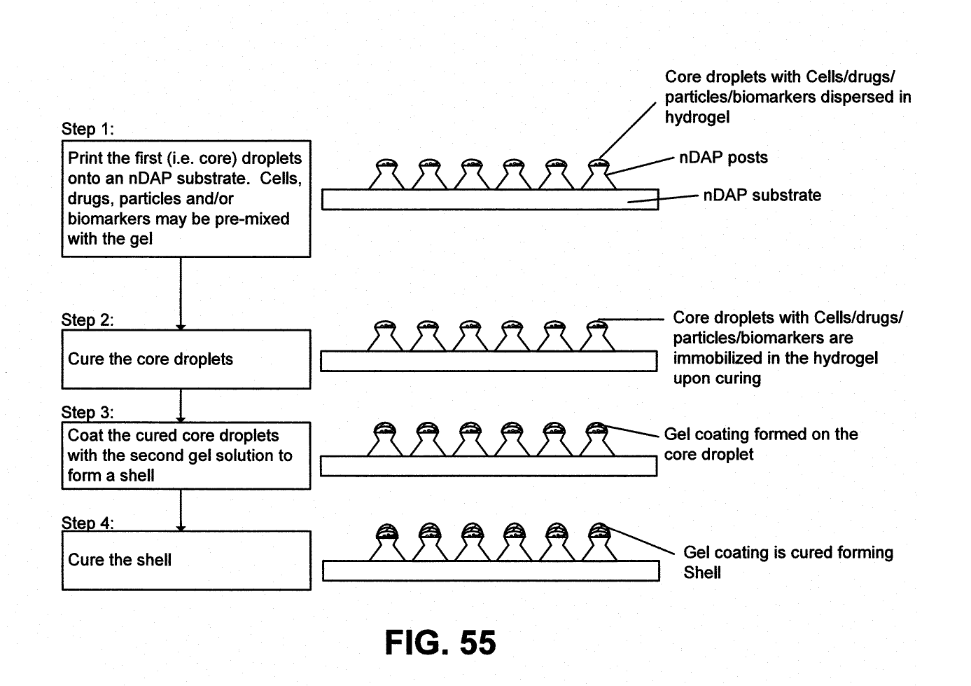

A flow diagram and schematics describing the process steps used to encapsulate cells in a core-shell hydrogel drop array are shown in FIG. 55. The first step is to dispense the core drops of the first hydrogel materials onto an nDAP substrate using either serial dispensing or parallel dispensing as described elsewhere in this specification. Cells/drugs/particles/biomarkers can be mixed with the hydrogel solution before dispensing. After dispensing, the hydrogel drop can be cured based on the curing mechanisms of the selected hydrogel materials as shown in the second step. In one example, an ion-cured hydrogel (such as sodium alginate) is used. After the alginate gel is printed on the nDAP surface, the gel drops are cured (e.g. immersed in an aqueous solution of CaCl₂, exposed to a fog/mist of aqueous CaCl₂ droplets, or premixed with particles of a calcium salt that slowly dissolves to release Ca⁺² ions. In another example, a temperature sensitive hydrogel (such as collagen based hydrogels, agarose and various artificial gels) is used. After dispensing, the core drop can be cured at a specific temperature within a high humidity environment to prevent the core droplet from evaporating. In another example, a light-sensitive hydrogel (such as a synthetic photo-crosslinkable polymer hydrogel) is used. After dispensing, the drop can be cured upon exposure to an adequate dose of light. In another example, a thixotropic hydrogel is used. After dispensing, the drop can be stored in a high humidity environment for a sufficient time to cure.

After the curing, the core gel drop is strongly anchored onto the nDAP lid, so that the cured core gel drop can be dipped into a second gel solution to form a coating on the outside of the drop as described in step 3. The coating thickness on the core drop can be easily adjusted by controlling the hydrogel concentration, surface tension as well as the dipping and withdrawing velocities. As with the core hydrogel, cells, drugs, particles, biomarkers, etc. can be premixed with the second (i.e. shell) hydrogel solution. In step 4, the thin layer of gel solution coated on the core drop can be cured based on the specific curing mechanisms of the hydrogel material used, as discussed for step 2 above.

The advantage of the core-shell structure is that cells (or other inclusions) contained within the core gel, are prevented from leaving the gel and entering the surrounding solution by the shell. For example, cells dispersed in a collagen hydrogel droplet are initially immobilized within the cross-linked gel. Over time, however, cells can cause the collagen to degrade such that the mobility of the cells in the gel increases and the cells can migrate out of the gel and into the surrounding solution (e.g. cell culture growth medium). By encapsulating the collagen with an alginate gel, the cells will not be able to escape, even if the collagen becomes a viscous liquid (as opposed to a crosslinked hydrogel).

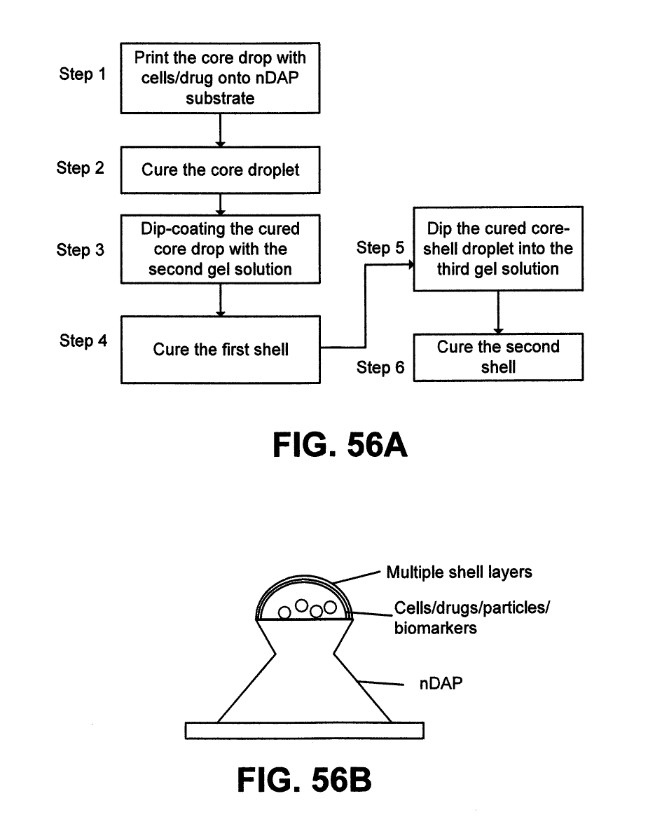

This innovation enables the formation of structures with multiple shells around a core. The process steps are shown in FIG. 56A. Steps 1 to 4 are the same as the process for forming a single shell in FIG. 55. Steps 3 and 4 are repeated (Steps 5 & 6) to make a second shell layer. This process can be repeated multiple times to make additional shells around a core. Cells, drugs, particles, and/or biomarkers can be premixed with the hydrogel that is used for the core as well as any or all of the shells as illustrated in FIG. 56B. Many combinations are possible. For example: cells can be dispensed in the core; beads that can bind cytokines are dispensed in the first shell and a drug is dispersed in the third shell.

Encapsulating Cells in Liquid Core-Shell Hydrogel Drop Arrays

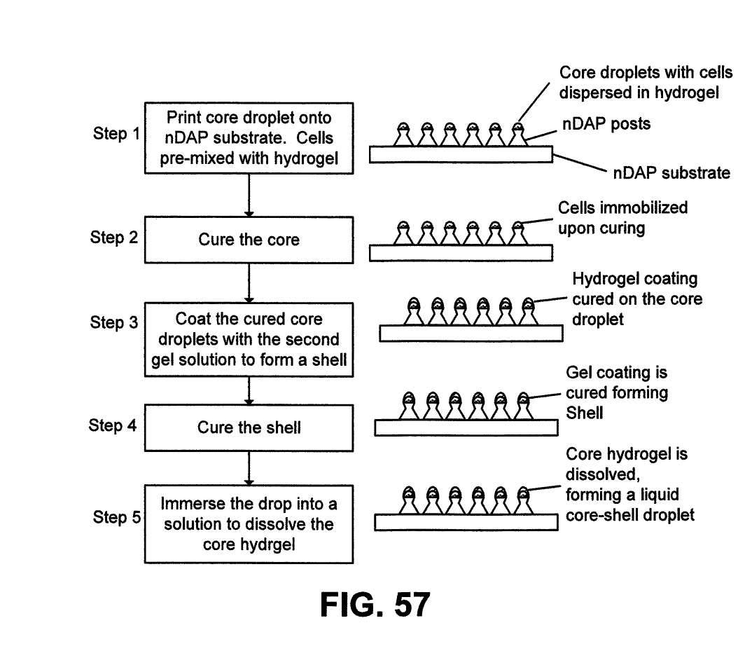

A third innovation enables the fabrication of core-shell droplets where the core is liquid, or of a sufficiently low crosslink density that cells can move freely within the core. One approach to fabricating this structure is by mixing a dissolvable material into the hydrogel that forms the core droplet. The process steps for this method are shown in FIG. 57; Steps 1-4 are the same as those shown previously in FIG. 55, FIG. 56A and FIG. 56B. After the stable, cured shell is formed in Step 4, the material in the core can be dissolved to form the liquid core-shell hydrogel drop as shown in Step 5 in FIG. 57.

The core drop could contain a small portion of dissolvable hydrogel materials, such as less than 1% in weight. Preferably, the dissolvable hydrogel materials can be biodegraded by cells, medium, or benign buffers. For example, 0.2% sodium alginate hydrogel can be used to form a stable core using a calcium ion curing agent and a collagen hydrogel can be used to form the shell on the alginate core. The alginate core can be fully dissolved by immersing the core-shell drop into a sodium citrate buffer. The sodium citrate can diffuse through the collagen shell and interact with the calcium forming crosslinks between alginate chains. The calcium ion is preferentially coordinated by the bidentate citrate ligand, freeing sodium ions to coordinate with each of the two carboxylic acid moieties on alginate chains that were formerly crosslinked together by calcium ions.

Similarly, dissolvable and nontoxic particles such as ice, sugar, metal oxides, metal carbonates, or biopolymers can be pre-mixed and dispensed with cells and the core hydrogel material. After the shell is cured, the particles can be dissolved to form a highly porous gel structure in which cells have a high mobility. By adjusting the concentration of hydrogel, as well as the concentration of particles, the mobility of cells within the hydrogel can be tuned to the desired value after the particles are dissolved.

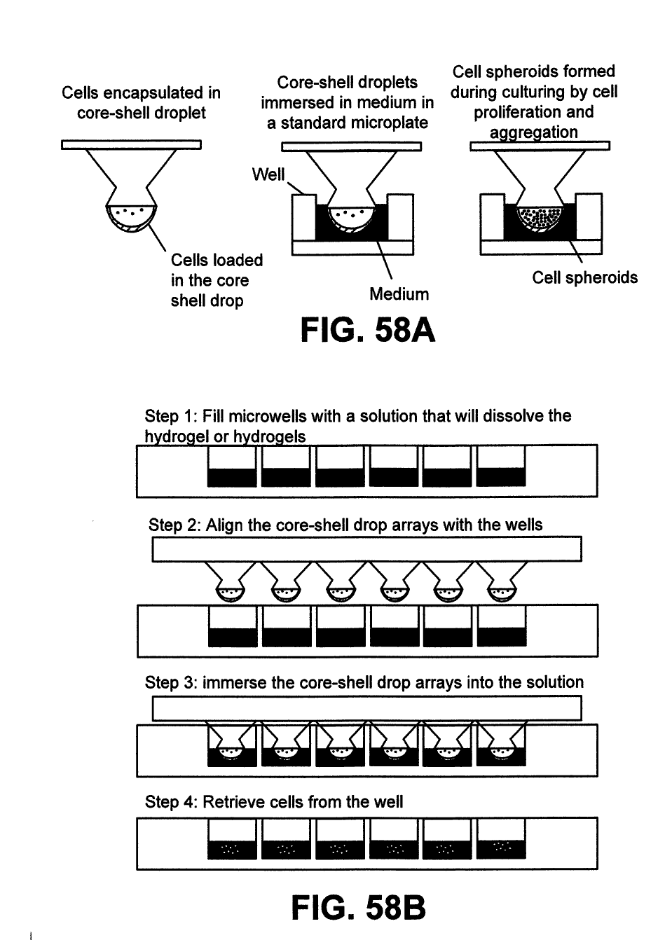

The core-shell as well as liquid core-shell hydrogel droplet arrays on nDAP substrates provide a suitable environment to form cell spheroids with specific sizes. The process is shown schematically in FIG. 58A. At first a certain number of cells are loaded in the core-shell drop. The core-shell drop can be immersed in medium to culture the cells for any length of time desired (hours to months). Cells in this system can be cultured for long periods as long as oxygen, nutrition as well as other compounds from the culturing medium can efficiently penetrate through the cured shell and migrate into the core. Because the core-shell droplets are anchored onto the nDAP lid, changing medium is very easy. To change medium, the nDAP lid is removed, the solution in the standard microplate is changed and/or replenished using standard pipetting operations, and the nDAP lid is replaced. No specific training is required; no centrifuging is needed to separate cells, and no cells are lost.

The spheroids size and cell densities can be adjusted by controlling the number of cell seeds, the volume of the core-shell drop, the viscosity and/or rigidity of the core hydrogel as well as the culturing time. The method described elsewhere in this specification, for encapsulating cells in liquid core-shell hydrogel drop arrays, may be especially well-suited for the formation of cell spheroids of uniform size. The high mobility of cells in a liquid core, or a core comprised of a highly porous hydrogel, may promote the formation of uniform cell spheroids.

Cells can be retrieved from the core-shell hydrogel droplets generated and anchored on the nDAP lid using solutions that can dissolve or degrade the hydrogel materials as shown in FIG. 58B. A microwell plate is first filled with an appropriate solution. For example, collagenase for collagen based hydrogels, or sodium citrate buffer for alginate based hydrogels. Then the core-shell drop arrays on nDAP lids are aligned with the wells and immersed into the solutions to fully or partially dissolve the hydrogel to release and retrieve the cells. After release, the cells can be used for other experiments, or analyzed using conventional cell techniques such as flow cytometry.

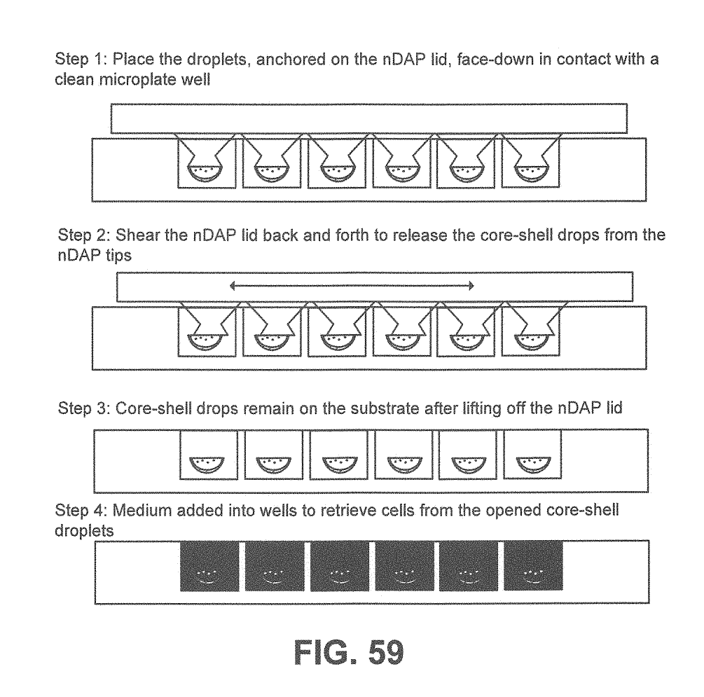

A novel method of mechanically opening the core-shell drops to retrieve cells has also been developed. The process is shown schematically in FIG. 59. The droplet arrays on the nDAP lid are placed in contact with a substrate, such as the bottom of a microplate, as shown in FIG. 59. The nDAP lid can then be translated horizontally to shear the core-shell drops off the nDAP tips as shown in FIG. 59. The gel droplets are transferred to the substrate because of the adhesive forces between the droplet hydrogel shell and the substrate. The core of the droplet is now exposed to the air. The nDAP lid is lifted away, leaving each core-shell droplet in a well as shown in FIG. 59. If the core is a liquid, then medium, buffer, or other fluids can be added into the wells, and cells can be easily dispersed and retrieved.

The advantage of this method, compared to the method described elsewhere in this specification, is that the outer cured gel need not be dissolved. This minimizes exposure of cells located in the core to citrate buffer or collagenase enzyme that may harm the cells or affect the assay.

Alternatively, the core of the droplet may be a cured hydrogel. In this case, a solution to dissolve the core hydrogel is added to the well. Again, no chemical is required to dissolve the shell.

### Example 12. Forming Core-Shell Hydrogels Arrays Using Sodium Alginate Hydrogels with Different Concentrations

Sodium alginate solutions (1% and 0.5% by weight) and calcium chloride solution (1% by weight) were made by dissolving sodium alginate powder (MP Biomedicals Inc) and calcium chloride powder in distilled water. Silver coated glass particles with an average diameter of 20 μm were used as a model for cells or particles. At first, drops of 0.5% sodium alginate containing 0.5% silver coated glass particles were dispensed manually onto a polystyrene (PS) lid made with nDAP structures. The PS lid had 96 posts with the same pitch as a standard 96 well microplates (e.g. a pitch of about 9 mm, i.e. 9±1 mm). When other plates are used (e.g. a 384 well plate or a 1,538 well plate) other pitches are used (e.g. 4.5±1 mm and 2.5±1 mm, respectively). In one embodiment the pitch is greater than 1 mm. In another embodiment, the pitch is greater than 5 mm. The tip of the posts (anvils) on the lid had a diameter of about 2 mm, and the volume of the core drop was about 1 uL. After dispensing arrays of core droplets of alginate, the drop arrays on the PS nDAP lid were immersed in 1% calcium solution for 30 seconds to cure and anchor the core droplets onto the lid. The silver coated glass particles were immobilized in the core droplets after curing. The droplet arrays were washed in DI water and dipped into the 1% alginate solution. A thin coating of 1% alginate was thus formed on the surface of the cured 0.5% alginate core droplets. The coated droplet arrays were then immersed into the 1% CaCl₂ solution for 30 seconds to cure the 1% alginate shell. The formed alginate core-shell hydrogel droplets were firmly anchored on the PS lid and could be stored in medium or other buffer solutions.

This example demonstrates that the invented method is suitable for making core-shell hydrogel drops using the same hydrogel materials with same curing mechanisms/conditions.

### Example 13. Forming Core-Multiple Shell Hydrogels Arrays Using Sodium Alginate Hydrogel Solutions with Different Concentrations

Alginate solutions and the silver coated glass particles were the same as those used in Example 12. The steps of forming the core and the first shell were also the same as described in Example 12. After forming the core and the first shell, the drop arrays were again washed with DI water and dipped into the 1% alginate solution, and cured in the 1% calcium solution for 30 seconds. These steps were repeated 10 times and the drop diameter increased significantly with each repetition, demonstrating the accumulation of shell layers upon each dip coating and curing step.

This example demonstrated that stable core-multiple shell hydrogel arrays can be made on the PS lid with nDAP structures by the invented method.

### Example 14. Forming Collagen Core-Alginate Shell Hydrogel Drops Encapsulating CaCO₃ Particles

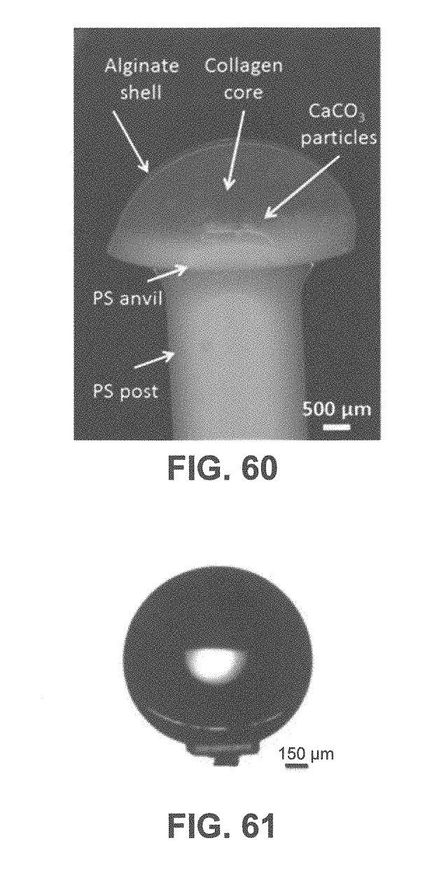

Temperature sensitive collagen hydrogel solution was used to make the core drop. CaCO₃ (Acros Organics 192721000) particles with a wide diameter range were used as a model for cells or other particles. CaCO₃ particles, 0.5%, were added into the collagen hydrogel solution and mixed while maintaining the temperature of the solution between 0-5° C. using an ice bag. The cold collagen-CaCO₃ mixture was dispensed onto the PS lid to form droplets with a volume of ˜1 μL as described in example 12 and example 13, and cured at 37° C. with high humidity for 5 min. The cured droplet arrays were then cooled and dipped into the 1% alginate solution. A stable coating of alginate was formed on the outside of each cured collagen droplet. The coated droplets were then immersed in the 1% calcium solution and cured for 30 seconds. After these steps, stable collage core/alginate shell hydrogel droplets were generated on the PS lid. A large droplet with a collagen core/alginate shell structure, made with this method, is shown in FIG. 60. A collagen core/alginate shell structure encapsulating CaCO₃ particles is depicted. The thickness of the alginate shell is approximately 150 μm.

This example demonstrates that the invented method is suitable for making stable core-shell hydrogel arrays using different hydrogel materials with different curing mechanisms used to cure each hydrogel.

The disclosed nDAP substrate can be used to not only anchor hydrogel droplets onto the surface, but can be used to form a core-shell structure using either the same, or different hydrogels for the core and the shell. The non-wetting properties of the nDAP design (i.e. overhanging top surface with concave re-entrant angle leading to the post) are important to keep the solutions on the anvil in a highly controllable and reproducible manner. Although the nDAP properties are important for ensuring reproducible volumes of solutions dispensed and strong adhesion between the hydrogel droplet and rigid (e.g. polymer) substrate, it may be possible to use a similar method on other, non-nDAP, substrates to form core-shell droplets with the desired properties described in this disclosure.

A first key innovation is a method to securely anchor both the core and shell hydrogel layers firmly to the substrate so that they can be repeatedly immersed into cell culture media, buffer solutions, or similar fluids without being prematurely dislodged. If the droplet-surface adhesion is lost before the end of the experiment, then the cells may be inadvertently released into the medium and lost when the medium is changed.

Also disclosed herein is a method to create core-shell droplets such that cells can freely move within a core while the droplets remain anchored on the nDAP posts. The barrier formed by the cured shell prevents the cells from leaving the core. Two methods are described for how the viscosity of the core can be modified to increase the ability of cells to move after the shell is cured. In one method, the curing reaction of the core hydrogel is reversed through the introduction of a chemical agent, such as a sodium citrate buffer (to scavenge calcium ions from a cured alginate gel) or a collagenase enzyme to degrade a cured collagen gel. In a second method, particles can be mixed with the hydrogel used to form the core droplet. The particles can be subsequently dissolved, or chemically degraded, after the shell is cured. Voids formed in the core gel by the dissolution or degradation of the included particles can leave isolated or interconnected pores in the gel through which cells can move.

A third innovation is a method to free the cells (or other particles) from core-shell hydrogel droplets. Two methods are described including the chemical dissolution/degradation of the shell hydrogel as well as the mechanical transfer of the droplets from an nDAP substrate onto a flat surface such as a standard microplate, exposing the core to the solution.

In all of these methods, one or multiple shell layers can be used as desired to achieve the necessary stability, adhesion and barrier properties.

Articles are also disclosed that can be prepared using these methods including: (1) A substrate with anchored hydrogel droplets such that inclusions (e.g. cells, drugs, particles, or biomarkers) are isolated in the core droplet and prevented from leaving the droplet by a shell composed of one or more layers. The droplets remain attached to the substrate through multiple days of cell culture and media changes. (2) A substrate with anchored hydrogel droplets such that cells can move easily within the core but are prevented from leaving the droplet by a shell. (3) A substrate with anchored hydrogel droplets such that inclusions in the core of the droplet may be released when desired and collected for further study.

The nDAP substrate can be used to detect attomole quantities of peptides and proteins using a standard MALDI-TOF instrument. In one approach, droplets of the protein dissolved in the appropriate matrix are dispensed on anvils using the robotic dispenser. The droplets are allowed to dry and then the nDAP plate is inserted into the MALDI-TOF instrument. To achieve the best performance, a MALDI-TOF holder is used that aligns the top of the nDAP anvils to the height used for standard MALDI-TOF measurements. The laser is then programmed to ablate the dried material on top of the anvils.

In one example, a series of solutions of the protein BSA (66.5 kDa) were made in matrix solution (4-HCCA saturated in 3:1:2 formic acid:water:isopropanol) with concentrations ranging from 10 μg/mL to 0.002 μg/mL. nDAP anvils with a diameter of 470 μm was first coated with a matrix solution (4-HCCA saturated in 1:2:1 0.1% TFA:ACN:water) and dried. Droplets with a volume of 19 nL were placed on the nDAP anvils, dried and measured using a Bruker microflex MALDI-TOF instrument. Peaks for BSA were observed at 66,500 (single charge), as well as 33,200 (doubly charged), and 22,200 (triply charged). All three peaks are clearly visible at concentrations down to 0.1 μg/mL, which is equivalent to a total of 2.66 attomoles dispensed on the anvil. The multiply charged peaks can be observed at even lower concentrations; the doubly and triply charged peaks are visible at concentrations as low as 0.01 μg/mL (0.27 attomoles total quantity of BSA). This is an excellent method for detecting extremely small quantities of peptides and proteins.

In a second example, a larger volume of solution can be deposited on the nDAP surface. Due to the superhydrophobic properties of the nDAP, droplets as large a several microliters can be dispensed on the surface using a hand-held pipette. Thus a droplet that is approximately 100 times larger than the nanoliter droplets described above can be used as the source. The droplet will evaporate, concentrating the peptide(s) and/or protein(s) onto one anvil. Thus extremely dilute solutions can be analyzed. In the case of BSA, a starting concentration as low as (0.0001 μg/mL or 100 picograms/mL), which is two orders of magnitude lower than the concentration detected in the previous example (0.01 μg/mL) can be detected given that the same number of moles of BSA (0.27 attomoles) will be present within the matrix on a single anvil. The use of smaller diameter anvils may give rise to even greater sensitivity.

Evaporation control. Several methods were evaluated to reduce the evaporation rate of dispensed nanoliter droplets. The use of a hydrogel in place of pure water was found to be the most effective. A solution of sodium alginate in DI water (amsbio LLC) was dispensed using the robotic rod dispenser and 600 μm nDAPs at 85% RH. After dispensing, the alginate was cured by exposing the slide to a fine mist of 5% aqueous calcium chloride generated by an ultrasonic mister for 30 sec. Cross-linking is rapid and the volume of the gel did not change after cure. The evaporation behavior of three different drops: pure DI water, uncured gel and cured gel were monitored using the same high speed camera system. The presence of gel decreases the evaporation rate compared to DI water, and cured gel results in the slowest rate. Essentially no evaporation was observed from a cured gel drop during the first 5 min at RH 85%.

These results demonstrate that using a cross-linked gel droplet significantly reduces the evaporation of nanoliter droplets at RH 85%. This stability is useful for dividing a microliter-sized droplet containing cells, antibiotics/antigens or other bioreagents into smaller nano droplets as well as depositing them onto the registered positions of an nDAP.

This experiment demonstrates that proteins can diffuse through the nanoliter gel droplets so long as the gel cross-link density is not excessive. Thus this approach will be suitable for studying cell expression/stimulation or conducting drug screening with proteins.

nVWP Fabrication: Nanoliter droplet virtual well microplates (nVWPs) were fabricated on standard microscope slides. The glass pedestals, measuring 500×500 μm in area and 100 μm thick, were placed by hand onto the printed PDMS posts. The height of the glass surface is 1.1 mm above the surface of the microscope slide and the pitch between adjacent pedestals is 1.2 mm. The area of each glass pedestal can be modified by dicing glass coverslips to different dimensions. The height and pitch of the anvils can be easily modified by programming the dispensing robot. Not every PDMS post need be populated with glass anvils.

Dispensing Processing: To demonstrate the effect of the glass edge, the dispensing processing was monitored using a high speed camera (Vision Research, Phantom V7.3). Initially, the dispensing rod, with a 10 μL droplet pinned to the bottom of the rod, descends and contacts the glass pedestal. The source drop quickly wets the top surface of the glass and the solid-liquid-vapor TCL spreads to the edge of the glass pedestal. The TCL is strongly pinned at this edge preventing the wetting of the side wall of the glass pedestals. As the source drop is retracted from the surface, the shape of the droplet changes; it becomes progressively more elongated because the TCL is immobilized at the edge of the glass. Eventually the droplet necks down sufficiently that it breaks, leaving a droplet with a specific volume and contact angle of about 80° remaining on the surface. The volume of the dispensed drop is determined by the glass perimeter and retraction velocity of the source droplet. If the droplet is allowed to evaporate after dispensing, the height of the droplet decreases, thereby decreasing the apparent contact angle. The liquid-solid contact area does not change because the TCL remains pinned at the edge of the glass pedestals throughout the entire evaporation process.

The volume of droplets dispensed onto the glass pedestals was calculated from the equation: V=πh (3a²+h²)/6, where “h” is the height of the spherical cap and “a” is the radius of the base of the cap. Values of h and a are measured from microscope images taken immediately after dispensing.

Due to the high energy barrier formed by the sharp edge of the glass pedestal, water droplets with static contact angles as high as 150° could be manually deposited on the pedestals. A 2 μL droplet, formed using an Eppendorf pipette and manually transferred to the glass pedestal, is shown in FIG. 61.

To further demonstrate that the liquid droplet contacts only the top surface of the pedestal and does not seep down the sidewalls, 28 μL of a fluorescently tagged protein solution (Dylight 488 labeled NeutrAvidin in PBS buffer) was manually dispensed on a glass pedestals of the nVWP. After drying, the location of the fluorescently labeled NeutrAvidin, as determined by confocal laser scanning microscopy (CLSM), was used to determine the extent of wetting on the glass. The fluorescent protein remained on the top surface of the glass pedestal, essentially no protein is observed on the sides or bottom surface of the glass. Restricting protein adsorption to the top surface demonstrates the strong pinning effect of the TCL at the edges of the glass pedestal. This means the solution and solute could be confined on the top surface during drying and thus increase the detection sensitivities especially for low concentration solutions.

The effect of surface chemistry on dispensing: The effect of glass pedestal surface chemistry on dispensing properties was studied by modifying the glass surfaces with either PEG to ensure that the surface is hydrophilic, or DMDCS to render the surface hydrophobic. On the hydrophilic surface, stable droplets (26.9±0.7 nL) were dispensed onto the PEGylated glass, similar to untreated clean glass. The solid-liquid-vapor TCL was strongly pinned by the sharp edge of the glass pedestal and so the transferred volume remained constant when the source drop was lifted off the surface. In contrast, droplets of a constant volume could not be transferred onto the hydrophobic glass surfaces because water could not be pinned at the edge of the glass. As a result, a small droplet (about 3 nL) was left behind at random positions on the glass pedestal. This experiment demonstrates that hydrophilic glass pedestals, which generate strong water-solid interactions, are essential for the surface controlled precise dispensing that is necessary to enable the nVWP technology.

Droplet precision achieved by manual and robotic dispensing: Manual and automated dispensing processes were evaluated to assess their impact on dispensed droplet precision. Cleaned glass pedestals were used with 10 μL DI water source droplets. Arrays of nanoliter droplets were dispensed onto the nVWP substrate and the volume of each drop was calculated based on microscope images. The average volume of the manually dispensed droplets was 27.8±2.03 nL yielding a dispense precision of 7.3%. The average contact angle of dispensed droplets was 77.9±3.4°. The precision for this simple manual process (±7.3% for 28 nL droplets) is comparable to the most advanced dispensing tools which require expensive and precise positioning systems.

Greater precision can be obtained by using a simple, low-cost robot to control the Z-axis motion of the dispensing rod. The robot was programmed to lower and raise the rod such that the source drop could contact the glass surface and be retracted at a specified velocity. At a lift-up velocity of 15 mm/sec, the average volume of the robotically dispensed droplets was 28.0±0.62 nL with a dispensing precision of better than 2.2% based on 10 samples. This is believed to be the highest precision ever reported for droplets of this small a volume. The average contact angle of the dispensed droplets was 78.0±0.67°. The Z-axis precision required to achieve this low percent error is a modest about ±50 μm; this value has seven times greater tolerance than required for pin dispensing. These results demonstrate that the nVWP substrate, coupled with a simple robot, provides excellent precision (2.2%) when dispensing droplets of about 30 nL while requiring modest positioning control.

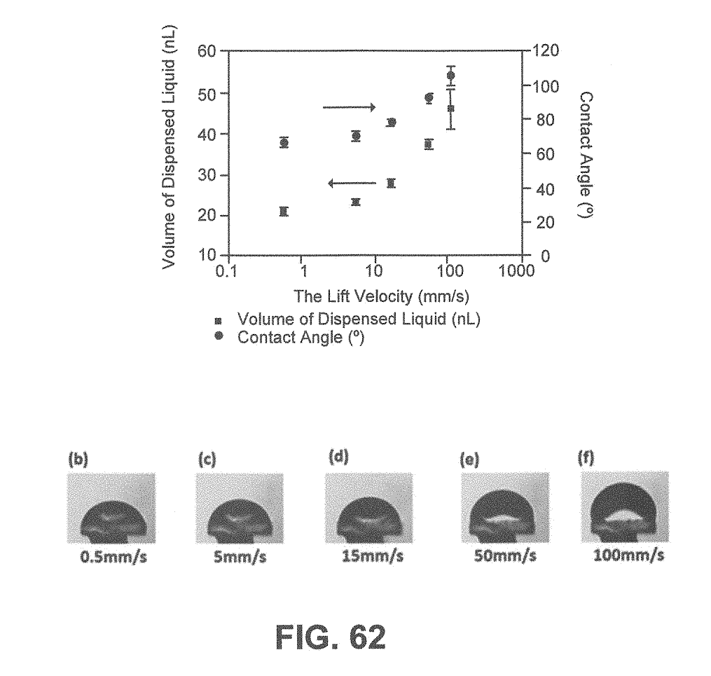

Referring to FIG. 62, the effect of lift-up velocity and source drop volume on dispense volume was determined. Two dispensing parameters, lift-up velocity and source droplet volume can be used to tune the volume of fluid dispensed onto a glass pedestal of fixed size and each parameter was studied with 10 samples. The lift-up velocity was studied over the range from 0.5 mm/sec to 100 mm/sec. The dispensed volume of the droplet increased slowly, from 21.1±0.5 nL to 23.3±0.3 nL, as the lift-up velocity was increased from 0.5 to 5 mm/sec. This result is consistent with the high repeatability obtained by manual dispensing, estimated to have a lift-up velocity in this same range. Increasing the lift-up velocity above this range, however, results in a faster, non-linear increase in dispense volume. When the lift-up velocity increased from 15 mm/sec to 100 mm/sec, the volume increased from 27.8±0.6 nL to 45.9±5.0 nL. The highest velocities result in a greater variance of dispense volume. This may be due to poor robotic movement reproducibility at high velocities or the effect of vibrations that these high velocities induce within the source drop.

Increasing the dispense volume on a given size glass pedestal results in an increase in apparent contact angle of the drop. Since water is pinned at the edge of the glass pedestal, increasing the volume results in a larger diameter droplet. Thus the contact angle increases with increasing droplet volume. The contact angle increases from 66.9° for a 21 nL droplet to over 105.8° for a 46 nL drop.

Overall, the volume of the dispense on a 500×500 μm pedestal can be controlled with good precision by a factor of 1.8, from 21 to 37 nL, by tuning the lift-up velocity over the range from 0.5 to 50 mm/sec. Increasing/decreasing the area of the glass pedestal will further extend the range of volumes that can be dispensed using the nVWP.

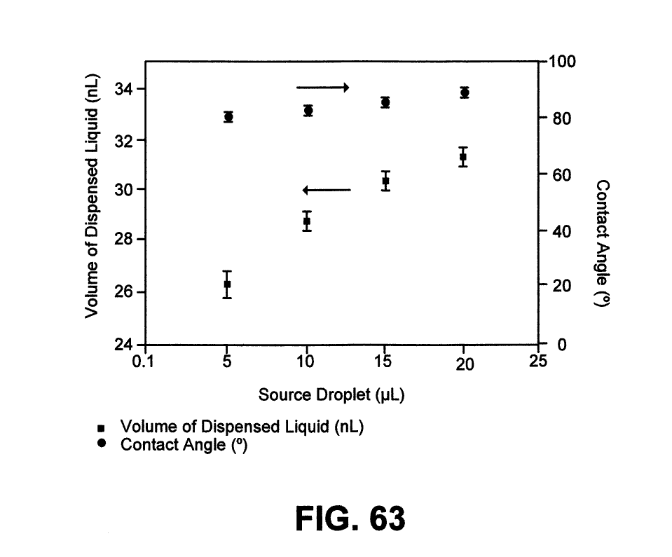

The source droplet volume can also be used to control the dispense volume. The effect of source drop volume on dispense volume and contact angle were determined on 10 samples measured for each parameter (see FIG. 63). The dispensed volume increases gradually from 26.2±0.6 nL to 31.3±0.4 nL with a concomitant increase of the contact angle from 81° to 89° as the source droplet volume was increased from 5 μL to 20 μL. At larger volumes, the effect of source drop volume became less pronounced. In all cases, the dispensed drop covered the whole surface of the glass pedestal and the TCL was pinned at the glass edge. Source drops less than 5 μL could not fully cover the edges of the glass pedestals during dispensing, resulting in non-reproducible liquid transfer. Because the dispense volume can be controlled by the source drop volume, care should be taken to periodically replenish the source drop when dispensing a large number of pedestals to maintain high precision. Overall, these results demonstrate that the source drop volume can be used to adjust the dispense volume, but this effect is weaker compared to the lift-up velocity.

The precision of the dispensed volume is maintained in the presence of proteins and/or surfactants in solution. To assess this effect, aqueous source drops containing 15 mg/mL of BSA in PBS buffer were dispensed on PEGylated glass pedestals. This high protein concentration results in a 4.8% higher dispensed volume compared with pure DI water on PEGylated glass pedestals. (28.2±1.2 nL vs 26.9±0.7 nL) based on measurement of 5 different samples. A dilute (0.1%) solution of TWEEN 80 did not affect (within the 2% uncertainty of the measurement) the dispensed volume of DI water on untreated clean glass pedestals. Both cases demonstrate that the process is robust, as surface tension has a small, but predictable effect on dispensed volume.

Detection limit and selectivity of fluorescently labeled proteins deposited on nVWP surfaces from nanoliter droplets: NeutrAvidin protein from Thermo Scientific was labeled using DYLIGHT® Antibody Labeling Kits following the procedure obtained from manufacturer. The labeled NeutrAvidin Dylight 488 had a concentration of 2 mg/mL in PBS buffer solution, which was used as a stock solution from which further dilutions were made. Droplets with a total volume of 28 nL and with specific concentrations (0.1 μg/mL, 0.5 μg/mL, 1 μg/mL, 5 μg/mL, 10 μg/mL, 20 μg/mL and 50 μg/mL) were automatically dispensed (i.e. using the robot) on the glass pedestals of nVWP and allowed to evaporate. The fluorescence intensity was then measured using a Zeiss Cell Observer Microscope using the same light source and exposure time (3 s) to enable comparisons between droplets/concentrations. Four droplet samples were measured for each concentration. Confocal microscopy with high resolution was used to detect protein absorbed onto the surfaces and edges of the glass pedestals.

Selective adsorption of NeutrAvidin from solution: A series of specific concentrations of NeutrAvidin DYLIGHT® 488 (0.01 μg/mL, 0.1 μg/mL, 0.5 μg/mL, 1 μg/mL and 5 μg/mL) in PBS buffer solution were prepared for the biotin-NeutrAvidin binding assay study. Fluorescein conjugated BSA (BSA, Alexa 555, from Invitrogen) was dissolved to prepare a 2 mg/mL stock solution in PBS buffer, and stored in the dark at 4° C. To study the selective binding of NeutrAviden, the stock solution of BSA Alexa 555 was diluted to 1 μg/mL using PBS buffer and mixed with different concentrations of labeled NeutrAvidin. Two samples for each NeutrAvidin concentration were prepared and analyzed. Before imaging, the nVWP was rinsed with PBS buffer twice to remove unbound protein. The fluorescence intensity of glass pedestals was imaged using a Zeiss Cell Observer Microscope (10× lens) with NeutrAvidin Dylight 488 detection wavelengths λₑₓ₍ₘₐₓ₎=494 nm, λₑₘ₍ₘₐₓ₎=518 nm.

MALDI-TOF detection of proteins on nVWP surfaces deposited from nanoliter droplets: A nVWP was directly fabricated onto a stainless steel MALDI plate (MSP 96 target, Bruker Daltonics). The MALDI plate was machined to create a recess 1.0 mm deep onto which the PDMS posts were printed. In this way, the top surface of the glass pedestals was at the same height as the MALDI plate surface. To deposit protein onto the glass pedestals, an ultrathin-layer sample preparation technique was used. The glass pedestals were first coated with 0.5 μL of a matrix solution (α-Cyano-4-hydroxycinnamic acid (4-HCCA) saturated in 1:2:1 0.1% trifluoroacetic acid:acetonitrile:water) and dried. Droplets of a mixture of NeutrAvidin and a second matrix solution (4-HCCA saturated in 3:1:2 formic acid:water:isopropanol) in a 1:10 ratio was dispensed onto the coated glass and dried. MALDI-TOF spectra were recorded on a Bruker Microflex MALDI-TOF instrument.

Detection of selectively adsorbed peptides on nickel-chelated treated glass nVWP surfaces: Nickel chelate resin coated glass pedestals (500×500×180 μm) were used to anchor KcsA ion channel proteins to selectively adsorb TX7335 peptide from snake venom. The KcsA ion channel, modified to contain a 6-his tag, was prepared in Tris-buffer (pH 7.5) with the surfactant n-decyl-β-D-maltopyranoside (DM) to form ion channel micelles. The 6-his tagged KcsA micelles were anchored onto the Ni-chelate-glass pedestals by depositing 1 μL droplets of the KcsA solution onto the pedestals, incubating for 5 min and removing the droplet with vacuum. After anchoring, excess ion channels were washed away by rinsing two times with DM buffer. Crude venom from the Eastern green mamba snake (Dendroaspis angusticeps) was pre-depleted of most non-specifically binding toxins with Ni-chelate resin, and the concentration was adjusted to 2 mg/mL. A 1 μL droplet of prepared venom solution was deposited onto the nickel-chelate coated glass pedestal of an nVWP and incubated for 2 min. Excess venom was washed away by rinsing with DM buffer for 6 times. A 1 μL droplet of matrix solution [4-HCCA saturated in 3:1:2 formic acid:water:isopropanol] was then added onto the surface and allowed to dry for MALDI-TOF detection.

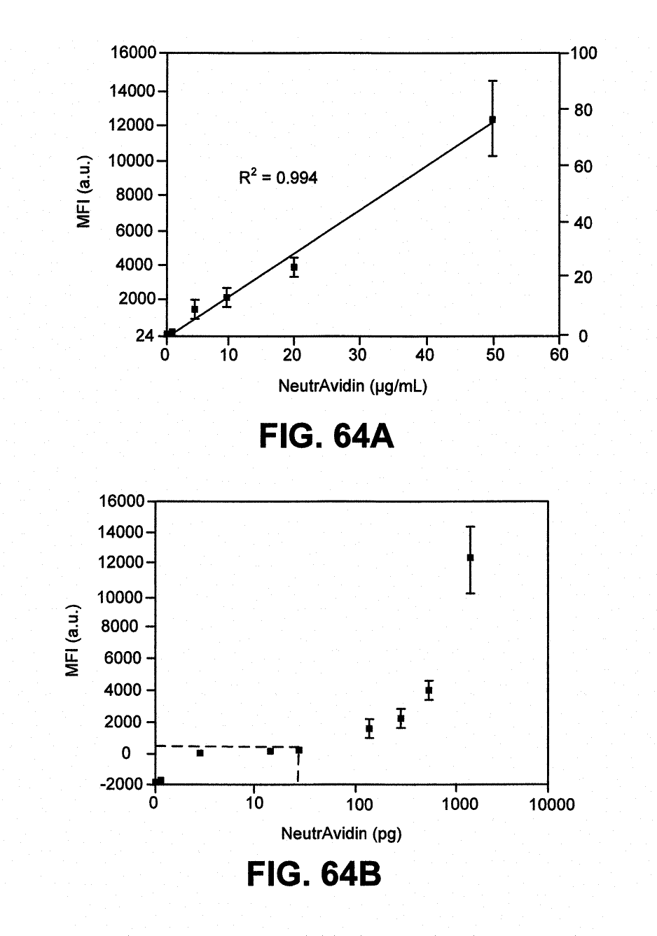

Detection sensitivity of proteins on nVWP by fluorescence microscopy and MALDI-TOF: Protein detection sensitivity by fluorescence microscopy: During evaporation of nanoliter droplets, the TCL remains pinned. As a result, the solution continues to wet the entire top surface of the glass pedestal as the solute molecules become increasingly more concentrated, eventually forming a continuous film. Thus the nVWP device improves detection sensitivity through the concentration of the solute onto spatially well-defined locations. To demonstrate this effect, nanoliter droplets (28 nL) of NeutrAvidin (MW=60 K) conjugated with Dylight 488 in PBS buffer solution were dispensed onto the glass pedestals and allowed to dry in the dark. FIG. 64A and FIG. 64B show that the fluorescence intensity increases linearly with increasing concentration of NeutrAvidin from 1 μg/mL to 50 μg/mL. Each data point was repeated on four different posts. Below 1 μg/mL, the fluorescence signal was too weak to be detected. The minimum amount of NeutrAvidin on the nVWP that could be detected was measured to be 28 pg (FIG. 64B), which corresponds to 467 attomoles of NeutrAvidin.

Protein detection sensitivity by MALDI-TOF: nVWPs fabricated on standard MALDI-TOF plates can be used to detect small quantities of biomolecules deposited onto the glass pedestal surfaces after evaporation of aqueous solutions. Samples were prepared using the ultrathin-layer sample preparation technique; After the initial matrix solution was dried, aliquots of a NeutrAvidin solution in PBS (0.5 mg/mL) were mixed with matrix solution and manually deposited on the glass pedestals forming 28 nL droplets that were allowed to dry. MALDI-TOF spectra obtained from the nVWP have the same peak positions compared with the sample deposited directly on the standard MALDI-TOF plate. The quantity of NeutrAvidin in the 28 nL droplets was varied from 252 to 0.126 pg (4,200 to 2.1 attomoles). Peaks at 14,300 and 7,090 which correspond to the singly and doubly charged NeutrAvidin monomer molecular ion (14,298 m/Z), were detected at quantities ranging from 4.2×10⁻¹⁵ to 4.2×10⁻¹⁷ moles, with a concomitant decrease in peak intensity. Each concentration was measured three times. The singly charged peak remains visible down to 4.2 attomol, but is no longer observed at 2.1 attomol. This sensitivity is comparable to the best values reported for MALDI-TOF and demonstrates the high sensitivity that can be achieved with nVWP using minute analyte amounts.

Selective adsorption and detection of NeutrAvidin from a protein mixture: The surface chemistry of the glass pedestals can be modified to selectively adsorb specific molecules, thus making the nVWP surfaces suitable as ELISA type assays. The well-known biotin-NeutrAvidin system was used as a model to study the selective adsorption of one protein and quantify the detection sensitivity using nVWP. Biotinylated and PEGylated glass pedestals were used to fabricate the nVWP; PEGylated glass pedestals were used as control. An array of 14 droplets of PBS solution containing Dylight 488 labeled Neutravidin at a concentration of 5 μg/mL were deposited onto the modified glass pedestals. To suppress droplet evaporation, the arrays were placed in a closed chamber containing a water reservoir. After incubation, the nVWP was rinsed thoroughly with DI water to remove unbound NeutrAvidin before drying. The specific binding of protein was analyzed as a function of incubation time at 26° using the Zeiss Cell Observer Microscope, maintaining the light source and exposure time constant. The mean florescence intensity (MFI) of the biotinylated surface was stronger than the PEGylated surface (370+/−103 vs 73+/−135 respectively) after 1.5 hours of incubation. However, this fluorescence signal was weak (MFI less than 400 a.u.); increasing the incubation time from 1.5 hour to 3 hours had no significant effect on the detected signal intensity. The weak fluorescence indicates limited binding of NeutrAvidin.

Without wishing to be bound to any particular theory, it is believed that the reaction is diffusion limited. Because of the small size of the droplets and the equilibrium established within the sealed chamber, there are neither thermal gradients nor evaporation that can drive convection within the droplet. Thus, the NeutrAvidin concentration near the biotinylated surface will become depleted. To maintain a constant concentration of NeutrAvidin near the surface that can react with biotin, convection within the droplet was promoted by inducing evaporation. An array of 0.5 μL droplets of Dylight 488 labeled NeutrAvidin with a concentration of 5 μg/mL was deposited on nVWP. Incubation was conducted under conditions that promoted rapid evaporation (26° C., 26% relative humidity) such that the droplet shrinks. The fluorescence intensity of the biotinylated surfaces increases rapidly with increasing incubation time from 1000 a.u. at 10 minutes to over 4500 a.u. after 19 mins. Longer times (greater than 19 mins) led to droplet evaporation and/or protein precipitation. The higher MFI values, and their time dependency indicates that the combined effect of convection and increasing NeutrAvidin concentration (due to evaporation of water) results in much faster reaction rates at the glass surface compared to the static droplet experiments. In contrast, protein adsorption on the PEGylated surface is limited. Over the 0-19 minute time span, the MFI on the PEGylated surface remains relatively low and constant. Each data point was measured on three different glass pedestals. This experiment demonstrates that the selective adsorption of proteins on nVWP can be significantly accelerated by introducing evaporation without increasing nonspecific adsorption. The minimum concentration of NeutrAvidin that can be detected from 0.5 μL droplets, is 0.1 μg/mL, which corresponded to a total of 800 attomoles of NeutrAvidin.

In many assays, numerous proteins are present in the same droplet that can complete with the target protein for adsorption sites on the surface. Thus the presence of these other proteins can reduce the sensitivity of the assay. To assess this effect, a series of protein solutions were prepared containing both NeutrAvidin Dylight 488 and BSA. The concentration of labeled NeutrAvidin in the 0.5 μL droplets was varied from 5 μg/mL to 0.01 μg/mL while the concentration of BSA was maintained at 1 μg/mL. After placement, the droplets were allowed to evaporate (26° C. and 26% RH) for 19 min, rinsed thoroughly with DI water, dried and stored in the dark until measured.

The high selectivity for NeutrAvidin on biotinylated glass pedestals was not affected by the presence of BSA. NeutrAvidin was selectively bound to biotin from solutions containing BSA with a minimum detectable NeutrAvidin concentration of 0.1 μg/mL (800 attomoles). This is the same detection threshold observed for NeutrAvidin alone with no BSA present. In summary, functionalized nVWP surfaces, combined with controlled evaporation, can quickly and sensitively detect the selective adsorption of proteins from complex solutions.

Selective binding of a snake venom peptide by an ion channel protein anchored on nVWP: Ion channels are attractive targets for drug discovery and the venom of scorpions, spiders and snails provide a veritable treasure trove of ion channel modulating peptides. To illustrate the power of the nVWP platform, an experiment was designed that highlights the advantages of working with nanoliter droplets of difficult to obtain natural products. Glass pedestals coated with nickel chelate resin were cut to size and fabricated into a nVWP surface directly on a MALDI-TOF plate. An ion channel protein, KcsA, modified to include a 6-his tag, was incorporated into a micelle and bound to the surface of the Ni-chelated resin coated glass pedestals through the 6-his tag. MALDI-TOF analysis confirmed the presence of the ion channel on the glass surface as two peaks specific to the ion channel protein at 6400 m/Z and 9550 m/Z were detected. A droplet (about 30 nL) of pre-depleted snake venom was manually dispensed onto the glass pedestal modified with the ion channel micelle and incubated for 2 min in air, followed by 6 rinses with DM buffer. A 1 μL droplet of matrix was added and allowed to dry. A MALDI-TOF analysis was conducted. Three peaks are observed: 6400 m/Z, which is due to the ion channel protein; 6680 m/Z, which is attributable to non-specifically bound peptides and; 7335 m/Z, which is attributed to a peptide that selectively binds to KcsA. This peptide is a toxin that has been shown to modify the function of the KcsA ion channel. To verify the non-specific vs specific binding of venom peptides, a control experiment was conducted where a droplet (about 40 nL) of pre-depleted venom was deposited on the Ni-chelated resin coated glass directly, without first biding the ion channel micelle. The MALDI-TOF spectrum shows a strong peak at 6680 m/Z demonstrating that the non-specific adsorption of peptides occurs on the Ni-chelate resin. Only a trace of TX7335 can be seen in this control.

Thus ion channel functionalized nVWPs provide an especially effect means for rapidly screening peptides from biologically important natural products, such as venoms, that are found in limited volumes. Less than 30 nL was required to identify the peptide that specifically binds to the KcsA ion channel. No labeling of the peptide was required.

This disclosure demonstrated that an array of small (500×500 μm) glass pedestals, nVWP, can be fabricated that enables the precise dispensing of nanoliter droplets. The sharp edges and hydrophilic properties of the glass cause the triple contact line (TCL) to be pinned, which is essential for precisely controlling the dispensed volumes. The local energy barrier at the sharp edges could effectively retain biofluids on the top of each glass pedestal without wetting the sidewalls; thus effectively concentrating the biomolecules for detection by fluorescence or MALDI-TOF. Droplet volumes can be controlled over a wide range (21-46 nL), with the same size glass pedestals (i.e. 500×500 μm), by adjusting the Z-axis lift-off velocity of the robot. The precision achieved for dispensing nanoliter droplets on the nVWP substrate by manual and automatically dispensing was ±7.3% and ±2.2%, respectively. Because all solute molecules in the nanoliter droplets are confined to the top surface of the pedestals, a detection limit of 467 attomole was achieved by fluorescence microcopy. The detection sensitivity could be further improved to 7.5 attomole when MALDI-TOF was used.

Modifying the surface chemistry of the glass pedestals further increases the functionality of the nVWP device. The glass surface can be biotinylated and PEGylated to selectively adsorb NeutrAvidin while minimizing non-specific adsorption of other proteins. NeutrAvidin was selectively adsorbed at levels as low as 800 attomoles in the presence of BSA as measured by fluorescence microscopy. The glass pedestal surface could also be coated with a nickel chelate resin onto which an ion channel protein (KcsA), incorporated into a micelle, was bound. An nVWP so prepared could selectively bind the peptide toxin found in snake venom (TX7335) that reacts with this ion channel. This peptide was detected by MALDI-TOF using only a about 30 nL drop of pre-depleted snake venom.

Precise control of dispense volume, coupled with chemical functionalization of the siloxane surface (e.g. a glass surface), provides an effective platform for detection of biomolecules as well as ELISA assays and label-free MALDI-TOF screening tests. The nVWP device may prove to be especially effective when sample volumes are limited to nanoliter quantities.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A nano-droplet plate comprising:
a rigid substrate with a planar surface;
a plurality of elongated posts extending from the planar surface, each elongated post separated from adjacent elongated posts by a pitch distance of at least 1 mm, each elongated post comprising:
a bottom end directly connected to the planar surface, the bottom end having a bottom diameter;
a top end opposite the bottom end, the top end comprising a planar top surface having a top diameter and an area of less than 50 square millimeters;
a middle section connecting the bottom end and the top end, the middle section having an upper half and a lower half proximate the top end and the bottom end, respectively, wherein the upper half contacts the planar top surface of the top end.

2. The nano-droplet plate as recited in claim 1, wherein the upper half has a continuously varying geometric angle (ψ) that begins at 0° and increases to 90°.

3. The nano-droplet plate as recited in claim 1, wherein the upper half has at least one geometric angle (ψ) greater than 0° and less than or equal to 90° degrees where the upper half contacts the planar top surface of the top end.

4. The nano-droplet plate as recited in claim 1, wherein the pitch distance is at least 5 mm.

5. The nano-droplet plate as recited in claim 1, wherein the plurality of elongated posts consists of 96 elongated posts and the pitch distance is 9±1 mm.

6. The nano-droplet plate as recited in claim 1, wherein the plurality of elongated posts consists of 384 elongated posts and the pitch distance is 4.5±1 mm.

7. The nano-droplet plate as recited in claim 1, wherein the plurality of elongated posts consists of 1,538 elongated posts and the pitch distance is 2.5±1 mm.

8. The nano-droplet plate as recited in claim 1, wherein the top diameter is less than the bottom diameter.

9. The nano-droplet plate as recited in claim 1, further comprising a droplet of a liquid atop each planar top surface, wherein a diameter of the droplet corresponds to the top diameter of the planar top surface such that no droplet contacts more than one planar top surface.

10. The nano-droplet plate as recited in claim 1, wherein the middle section comprises a middle diameter and the middle diameter is less than both the top diameter and the bottom diameter.

11. The nano-droplet plate as recited in claim 1, wherein the middle section comprises a middle diameter and the middle diameter is less than the top diameter but greater than the bottom diameter.

12. The nano-droplet plate as recited in claim 1, the rigid substrate, the elongated posts and the planar top surface consist of a thermoplastic polymer.

13. The nano-droplet plate as recited in claim 1, the rigid substrate, the elongated posts and the planar top surface consist of a thermoplastic polymer selected from the group consisting of polystyrene, polyolefin, polycarbonate and acrylonitrile butadiene styrene.

14. The nano-droplet plate as recited in claim 1, the rigid substrate, the elongated posts and the planar top surface are monolithic.

15. A cell culture system comprising:
a nano-droplet plate comprising:
a rigid substrate with a planar surface;
a plurality of elongated posts extending from the planar surface, each elongated post separated from adjacent elongated posts by a pitch distance of at least 1 mm, each elongated post comprising:
a bottom end directly connected to the planar surface, the bottom end having a bottom diameter;
a top end opposite the bottom end, the top end comprising a planar top surface having a top diameter and an area of less than 50 square millimeters;
a middle section connecting the bottom end and the top end, the middle section having an upper half and a lower half proximate the top end and the bottom end, respectively; wherein the upper half contacts the planar top surface of the top end;
a well microplate comprising a plurality of wells, wherein the ratio of wells to elongated posts is 1:1 and the pitch distance is equal to a center-to-center well spacing of the plurality of wells.

16. The cell culture system as recited in claim 15, further comprising a droplet of a liquid atop each planar top surface, wherein a diameter of the droplet corresponds to the top diameter of the planar top surface such that no droplet contacts more than one planar top surface.

17. The cell culture system as recited in claim 16, wherein the well microplate comprises a liquid medium in each well of the plurality of wells and the nano-droplet plate is disposed above the well microplate such that each elongated post is immersed the liquid medium in a corresponding well in the plurality of wells.

18. The cell culture system as recited in claim 16, wherein each well in the plurality of wells comprises an optically transparent bottom surface such that an internal volume of each well can be optically imaged through the optically transparent bottom surface.

19. The cell culture system as recited in claim 16, wherein each of the elongated posts comprises as least one channel in the planar top surface that connects the planar top surface to a corresponding internal volume of a respective well in the plurality of wells.

20. The cell culture system as recited in claim 16, wherein the droplet comprises a cross-linked hydrogel.

21. The cell culture system as recited in claim 16, wherein the droplet further comprises a biological cell disposed therein.

22. The cell culture system as recited in claim 16, wherein the droplet further comprises a plurality of biological cells disposed therein, the plurality of biological cells forming a tissue.

23. The cell culture system as recited in claim 16, wherein the droplet comprises at least one layer of hydrogel containing biological cells.
